(19) United States
(12) Patent Application Publication
(10) Pub. No.: US 2014/0357655 A1
(43) Pub. Date: Dec. 4, 2014

(54) SPIROINDOLINE DERIVATIVES AS GONADOTROPIN- RELEASING HORMONE RECEPTOR ANTAGONISTS

(71) Applicant: Bayer Intellectual Property GmbH, Monheim (DE)

(72) Inventors: Olaf Panknin, Berlin (DE); Stefan Bäurle, Berlin (DE); Sven Ring, Jena (DE); Wolfgang Schwede, Glienicke (DE); Wilhelm Bone, Berlin (DE); Katrin Nowak-Reppel, Berlin (DE); Eckhard Bender, Langenfeld (DE); Reinhard Nubbemeyer, Berlin (DE); Mark Jean Gnoth, Mettmann (DE)

(21) Appl. No.: 14/371,312

(22) PCT Filed: Jan. 15, 2013
(86) PCT No.: PCT/EP2013/050676

§ 371 Date: Jul. 9, 2014

(30) Foreign Application Priority Data
Jan. 16, 2012 (EP) 12151291.7

(51) Int. Cl. C07D 495/10; C07D 491/107

(52) U.S. Cl. CPC C07D 495/10; C07D 491/107
USPC 514/278; 546/15; 548/410; 514/409; 548/246; 514/380; 548/216; 514/377; 548/147; 514/370

(57) ABSTRACT

Spiroindoline derivatives, process for their preparation and pharmaceutical compositions thereof, their use for the treatment and/or prophylaxis of diseases, and their use for the manufacture of medicaments for the treatment and/or prophylaxis of diseases, especially sex-hormone-related diseases in both men and women, in particularly those selected from the group of endometriosis, uterine fibroids, polycystic ovarian disease, hirsutism, precocious puberty, gonadal steroid-dependent neoplasia such as cancers of the prostate, breast and ovary, gonadotrope pituitary adenomas, sleep apnea, irritable bowel syndrome, premenstrual syndrome, benign prostatic hypertrophy, contraception and infertility (e.g., assisted reproductive therapy such as in vitro fertilization). The present application relates in particular to spiroindoline derivatives as gonadotropin-releasing hormone (GnRH) receptor antagonists.

### TECHNICAL FIELD

The present invention refers to spiroindoline derivatives as gonadotropin-releasing hormone (GnRH) receptor antagonists, pharmaceutical compositions containing a spiroindoline derivative according to the invention and methods of treating disorders by administration of a spiroindoline derivative according to invention to a mammal, particularly a human, in need thereof.

### BACKGROUND ART

Gonadotropin-releasing hormone (GnRH) is a decapeptide (pGlu-His-Trp-Ser-Tyr-Gly-Leu-Arg-Pro-Gly-NH₂) released from the hypothalamus, also known as luteinizing hormone-releasing hormone (LHRH). GnRH acts on the pituitary gland to stimulate the biosynthesis and release of luteinizing hormone (LH) and follicle-stimulating hormone (FSH). LH released from the pituitary gland is responsible for the regulation of gonadal steroid production in both genders and late ovarian follicle development and ovulation in female mammals, FSH regulates spermatogenesis in males and early follicular development in females. Thus GnRH plays a key role in human reproduction.

As a consequence of its biological significance, synthetic antagonists and agonists to GnRH have been the center of several research activities, particularly in the field of endometriosis, uterine leiomyoma (fibroids), prostate cancer, breast cancer, ovarian cancer, prostatic hyperplasia, assisted reproductive therapy and precocious puberty.

For example, peptidic GnRH agonists, such as leuprorelin (pGlu-His-Trp-Ser-Tyr-d-Leu-Leu-Arg-Pro-NHEt), are described for the use in the treatment of such conditions (The Lancet 2001, 358, 1793-1803; Mol. Cell. Endo. 2000, 166, 9-14). Said agonists initially induce the synthesis and release of gonadotropins, by binding to the GnRH receptor on the pituitary gonadotrophic cells (flare-up′). However, chronic administration of GnRH agonists reduces gonadotropin release from the pituitary and results in the down-regulation of the receptor, with the consequence of suppressing sex steroidal hormone production after some period of treatment.

GnRH antagonists, on the contrary, are supposed to suppress gonadotropins from the onset, offering several advantages, in particular a lack of side effects associated with the flare up seen under GnRH superagonist treatment. Several peptidic antagonists with low histamine release potential are known in the art. Said peptidic products show low oral bioavailability which limits their clinical use.

The state of the art involves a number of nonpeptidic compounds for use as GnRH receptor antagonists, for example in WO2011076687, WO05007165, WO03064429 and WO04067535. Although intensive research has been driven for more than 15 years aiming at non-peptidic GnRH antagonists, none of them succeeded so far to reach the market.

Nevertheless, effective small molecule GnRH receptor ligands, especially compounds which are active as antagonists as well as pharmaceutical compositions containing such GnRH receptor antagonists and methods relating to the use thereof to treat, for example, sex-hormone-related conditions, in particular for the treatment of leiomyoma are still highly required in the pharmaceutical field.

The spiroindoline derivatives according to the present invention aim to fulfill such unmet need, and provide at the same time further advantages over the known art.

Spiroindoline derivatives are known in the art as pharmaceutically active ingredients and in the cropscience field as insecticides but their activity as GnRH receptor antagonists has not been described as far.

The document WO0066554 describes generic indolines as potential PR antagonists.

The document US200663791, page 20, describes the synthesis of a nitroindoline by condensing an aldehyde and a phenylhydrazine under acidic conditions (Fischer indole synthesis) and subsequent reduction of the indolenine intermediate.

Liu et al. describes the synthesis of a spirotetrahydropyrane in a similar manner in a one-pot reaction (Tetrahedron 2010, 66, 3, 573-577).

The document WO10151737, page 224, describes the synthesis of an indolenine mixture in an analogous Fischer indole synthesis by condensing an aldehyde with a phenylhydrazine.

The document WO06090261, pp. 67-68, describes the synthesis of a spiropiperidine via Fischer indole synthesis and subsequent addition of a Grignard reagent to the indolenine intermediate.

The document WO08157741, pp. 41-42, describes the synthesis of a spiropiperidine starting from an oxindole precursor via Grignard addition and subsequent deoxygenation.

The document WO9315051 discloses a generic oxindole as potential vasopressin/oxytocin antagonists.

Further spiroindoline derivatives with pharmaceutical properties were disclosed for example in the documents WO199429309, WO199964002 and WO200247679.

### DISCLOSURE OF THE INVENTION

The aim of the present invention is to provide gonadotropin-releasing hormone (GnRH) receptor antagonists, as well as the methods for their preparation and use, and pharmaceutical compositions containing the same.

In particular, the present invention relates to compounds according to Formula (I)

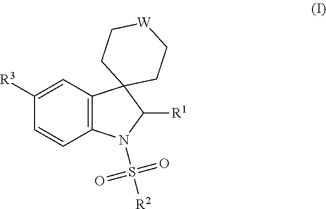

in which
W is selected from the group consisting of O, S(O)ₓ with x=0, 1 or 2;
R¹ is selected from the group consisting of hydrogen, C₁-C₆-alkyl,
C₃-C₁₀-cycloalkyl, C₂-C₆-alkenyl, C₂-C₆-alkynyl, aryl, hydroxy-C₁-C₆-alkyl; C₁-C₆-alkoxy-C₁-C₆-alkyl;
R² is an aryl or heteroaryl group which can be unsubstituted or substituted one to three times with a group R⁴ selected from a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, C(O)NH₂, C(O)NH—C₁-C₆-alkyl, C(O)N(C₁-C₆-alkyl)₂ in which the two alkyl groups are independent from each other, CN;
R³ is selected from the group consisting of C(O)N(R^{5a})(R^{5b}), N(H)C(O)R⁶, N(H)C(O)N(R^{5a})(R^{5b}), or N(H)C(O)OR⁷ and
R^{5a}, R^{5b} and R⁶ are selected, independently from one another, from the group consisting of hydrogen, C₁-C₆-alkyl, C₁-C₆-haloalkyl, hydroxy-C₁-C₆-alkyl; C₂-C₆-alkenyl, C₂-C₆-alkynyl, C₁-C₆-alkoxy-C₁-C₆-alkyl, C₃-C₁₀-cycloalkyl, C₃-C₁₀-cycloalkyl-C₁-C₆-alkylen-, aryl, aryl-C₁-C₆-alkylen-, aryl-cyclopropyl, heteroaryl, heteroaryl-C₁-C₆-alkylen-, in which said cycloalkyl, aryl, heteroaryl groups are optionally substituted up to three times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other;
R⁷ is selected from the group consisting of C₁-C₆-alkyl, C₁-C₆-haloalkyl, hydroxy-C₁-C₆-alkyl, C₂-C₆-alkenyl, C₂-C₆-alkynyl, C₁-C₆-alkoxy-C₁-C₆-alkyl, C₃-C₁₀-cycloalkyl, C₃-C₁₀-cycloalkyl-C₁-C₆-alkylen, aryl, aryl-C₁-C₆-alkylen-, heteroaryl, or heteroaryl-C₁-C₆-alkylen- in which said cycloalkyl, aryl, heteroaryl group is optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

A particular form of the invention refers to the compounds according to Formula (Ia)

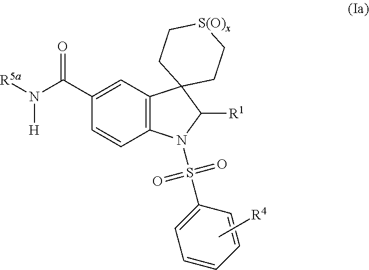

in which x=0, 1 or 2;
R¹ is selected from the group consisting of C₁-C₆-alkyl, C₁-C₆-cycloalkyl, alkenyl;
R⁴ is halogen, hydroxy, C₁-C₆-alkyl, C₁-C₆-alkoxy,
C₁-C₆-haloalkoxy, C(O)OH, C(O)O—C₁-C₆-alkyl, C(O)NH₂, C(O)N(C₁-C₆-alkyl)₂ in which the two alkyl groups are independent from each other, CN;
R^{5a} is C₃-C₁₀-cycloalkyl, C₃-C₁₀-cycloalkyl-C₁-C₆-alkylen-, aryl, aryl-C₁-C₆-alkylen-, heteroaryl, heteroaryl-C₁-C₆-alkylen-, in which said cycloalkyl, aryl, heteroaryl groups are optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

A further particular form of the invention refers to the compounds according to Formula (Ib)

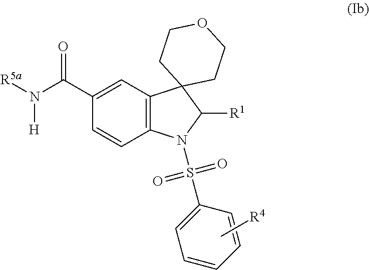

wherein R¹ is selected from the group consisting of C₁-C₆-alkyl, C₁-C₆-cycloalkyl, alkenyl;
R⁴ is halogen, hydroxy, C₁-C₆-alkyl, C₁-C₆-alkoxy,
C₁-C₆-haloalkoxy, C(O)OH, C(O)O—C₁-C₆-alkyl, C(O)NH₂, C(O)N(C₁-C₆-alkyl)₂ in which the two alkyl groups are independent from each other, CN;
R^{5a} is C₃-C₁₀-cycloalkyl, C₃-C₁₀-cycloalkyl-C₁-C₆-alkylen-, aryl, aryl-C₁-C₆-alkylen-, heteroaryl, heteroaryl-C₁-C₆-alkylen-, in which said cycloalkyl, aryl, heteroaryl groups are optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

Compounds according to the invention are the compounds of the formula (I), (Ia), (Ib) and the salts, solvates and solvates of the salts thereof, the compounds which are encompassed by formula (I), (Ia), (Ib) and are of the formulae mentioned hereinafter, and the salts, solvates and solvates of the salts thereof, and the compounds which are encompassed by formula (I), (Ia), (Ib) and are mentioned hereinafter as exemplary embodiments, and the salts, solvates and solvates of the salts thereof, insofar as the compounds encompassed by formula (I), (Ia), (Ib) and mentioned hereinafter are not already salts, solvates and solvates of the salts.

Hydrates of the compounds of the invention or their salts are stoichiometric compositions of the compounds with water, such as, for example, hemi-, mono-, or dihydrates.

Solvates of the compounds of the invention or their salts are stoichiometric compositions of the compounds with solvents.

Solvates which are preferred for the purposes of the present invention are hydrates.

Salts for the purposes of the present invention are preferably pharmaceutically acceptable salts of the compounds according to the invention (for example, see S. M. Berge et al., “Pharmaceutical Salts”, J. Pharm. Sci. 1977, 66, 1-19).

Pharmaceutically acceptable salts include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, maleic, fumaric, benzoic, ascorbic, succinic, acetic, trifluoroacetic, oxalic, propionic, tartaric, salicylic, citric, gluconic, lactic, mandelic, cinnamic, aspartic, stearic, palmitic, glycolic, and glutamic acid.

Pharmaceutically acceptable salts also include salts of customary bases, such as for example and preferably alkali metal salts (for example sodium, lithium and potassium salts), alkaline earth metal salts (for example calcium and magnesium salts), and ammonium salts derived from ammonia or organic amines, such as illustratively and preferably ethylamine, diethylamine, triethylamine, ethyldiisopropylamine, monoethanolamine, diethanolamine, triethanolamine, dicyclohexylamine, dimethylaminoethanol, benzylamine, dibenzylamine, N-methylmorpholine, N-methylpiperidine, dihydroabietyl-amine, arginine, lysine, and ethylenediamine.

Also encompassed are salts which are themselves unsuitable for pharmaceutical uses but can be used for example for isolating or purifying the compounds of the invention.

The present invention additionally encompasses prodrugs of the compounds of the invention. The term “prodrugs” encompasses compounds which themselves may be biologically active or inactive, but are converted during their residence time in the body into compounds of the invention (for example by metabolism or hydrolysis).

The present invention includes all possible stereoisomers of the compounds of the present invention as single stereoisomers, or as any mixture of said stereoisomers, e.g. R- or S-isomers, or E- or Z-isomers, in any ratio.

All isomers, whether separated, pure, partially pure, or in racemic mixture, of the compounds of this invention are encompassed within the scope of this invention. The purification of said isomers and the separation of said isomeric mixtures may be accomplished by standard techniques known in the art. For example, diastereomeric mixtures can be separated into the individual isomers by chromatographic processes or crystallization, and racemates can be separated into the respective enantiomers either by chromatographic processes on chiral phases or by resolution.

If the compounds of the invention may occur in tautomeric forms, the present invention encompasses all tautomeric forms.

Unless otherwise stated, the following definitions apply for the substituents and residues used throughout this specification and claims. The particularly named chemical groups and atoms (for example fluorine, methyl, methyloxy and so on) should be considered as particular forms of embodiment for the respective groups in compounds according to the invention.

The term “halogen atom” or “halo” is to be understood as meaning a fluorine, chlorine, bromine or iodine atom.

The term “C₁-C₆-alkyl” is to be understood as preferably meaning a linear or branched, saturated, monovalent hydrocarbon group having 1, 2, 3, 4, 5 or 6 carbon atoms, e.g. a methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2 or 3 carbon atoms (“C₁-C₃-alkyl”), methyl, ethyl, n-propyl- or iso-propyl.

The term “C₁-C₆-haloalkyl” is to be understood as preferably meaning a linear or branched, saturated, monovalent hydrocarbon group in which the term “C₁-C₆-alkyl” is defined supra, and in which one or more hydrogen atoms is replaced by a halogen atom, in the same way or differently, i.e. one halogen atom being independent from another.

Particularly, said halogen atom is F. Said C₁-C₆-haloalkyl group is, in particular —CF₃, —CHF₂, —CH₂F, —CF₂CF₃, —CF₂CH₃, or —CH₂CF₃.

The term “C₁-C₆-alkoxy” is to be understood as preferably meaning a linear or branched, saturated, monovalent, hydrocarbon group of formula —O-alkyl, in which the term “alkyl” is defined supra, e.g. a methoxy, ethoxy, propoxy, isopropoxy, butoxy, isobutoxy, tert-butoxy, sec-butoxy, pentyloxy, isopentyloxy, or hexyloxy group, or an isomer thereof.

The term “C₁-C₆-haloalkoxy” is to be understood as preferably meaning a linear or branched, saturated, monovalent C₁-C₆-alkoxy group, as defined supra, in which one or more of the hydrogen atoms is replaced, in the same way or differently, by a halogen atom. Particularly, said halogen atom is F. Said C₁-C₆-haloalkoxy group is, for example, —OCF₃, —OCHF₂, —OCH₂F, —OCF₂CF₃, or —OCH₂CF₃.

The term “C₁-C₆-alkoxy-C₁-C₆-alkyl” is to be understood as preferably meaning a linear or branched, saturated, monovalent alkyl group, as defined supra, in which one or more of the hydrogen atoms is replaced, in the same way or differently, by a C₁-C₆-alkoxy group, as defined supra, e.g. methoxyalkyl, ethoxyalkyl, propoxyalkyl, isopropoxyalkyl, butoxyalkyl, isobutoxyalkyl, tert-butoxyalkyl, sec-butoxyalkyl, pentyloxyalkyl, isopentyloxyalkyl, hexyloxyalkyl group, in which the term “C₁-C₆-alkyl” is defined supra, or an isomer thereof.

The term “C₁-C₆-haloalkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, in the same way or differently, by a halogen atom.

Particularly, said halogen atom is F. Said C₁-C₆-haloalkoxy-C₁-C₆-alkyl group is, for example, —CH₂CH₂OCF₃, —CH₂CH₂OCHF₂, —CH₂CH₂OCH₂F, —CH₂CH₂OCF₂CF₃, or —CH₂CH₂CH₂CF₃.

Alkylcarbonyl in general represents a straight-chain or branched alkyl radical having 1 to 4 carbon atoms which is bonded via a carbonyl group to the rest of the molecule. Non-limiting examples include acetyl, propionyl, butyryl, isobutyryl, pivaloyl.

Alkoxycarbonylamino illustratively and preferably represents methoxycarbonylamino, ethoxy-carbonylamino, propoxycarbonylamino, isopropoxycarbonylamino, butoxycarbonylamino and tert-butoxycarbonylamino.

Alkoxycarbonyl illustratively and preferably represents methoxycarbonyl, ethoxycarbonyl, propoxycarbonyl, isopropoxycarbonyl, butoxycarbonyl and tert-butoxycarbonyl.

Alkylsulfonyl in general represents a straight-chain or branched alkyl radical having 1 to 4 carbon atoms which is bonded via a sulfonyl (—SO₂—) group to the rest of the molecule. Non-limiting examples include methylsulfonyl, ethylsulfonyl, propylsulfonyl, isopropylsulfonyl, butylsulfonyl, tert-butylsulfonyl.

S-Alkylsulfonimidoyl in general represents a straight-chain or branched alkyl radical having 1 to 4 carbon atoms which is bonded via a sulfonimidoyl [—S(═O)(═NH)—] group to the rest of the molecule and which is attached to the sulfur atom of that group. Non-limiting examples include S-methylsulfonimidoyl, S-ethylsulfonimidoyl, S-propylsulfonimidoyl, S-isopropylsulfonimidoyl, S-butylsulfonimidoyl, S-tert-butylsulfonimidoyl. Monoalkylamino in general represents an amino radical having one alkyl residue attached to the nitrogen atom. Non-limiting examples include methylamino, ethylamino, propylamino, isopropylamino, butylamino, tert-butylamino. The same applies to radicals such as monoalkyl-aminocarbonyl.

Dialkylamino in general represents an amino radical having two independently selected alkyl residues attached to the nitrogen atom. Non-limiting examples include N,N-dimethylamino, N,N-diethylamino, N,N-diisopropylamino, N-ethyl-N-methylamino, N-methyl-N-propylamino, N-isopropyl-N-propylamino, N-tert-butyl-N-methylamino. The same applies to radicals such as dialkylaminocarbonyl.

Monoalkylaminocarbonyl illustratively and preferably represents methylaminocarbonyl, ethyl-aminocarbonyl, propylaminocarbonyl, isopropylaminocarbonyl, butylaminocarbonyl and tert-butylaminocarbonyl.

Dialkylaminocarbonyl illustratively and preferably represents N,N-dimethylaminocarbonyl, N,N-diethylaminocarbonyl, N,N-diisopropylaminocarbonyl, N-ethyl-N-methylaminocarbonyl, N-methyl-N-propylaminocarbonyl, N-isopropyl-N-propylaminocarbonyl and N-tert-butyl-N-methyl-aminocarbonyl.

Alkylcarbonylamino in general represents a straight-chain or branched alkyl radical having 1 to 4 carbon atoms which is bonded via a carbonylamino (—C(═O)—NH—) group to the rest of the molecule and which is attached to the carbon atom of that group. Non-limiting examples include acetylamino, propionylamino, butyrylamino, isobutyrylamino, pivaloylamino.

The term “C₂-C₆-alkenyl” is to be understood as preferably meaning a linear or branched, monovalent hydrocarbon group, which contains one or more double bonds, and which has 2, 3, 4, 5, 6 carbon atoms, particularly 2 or 3 carbon atoms (“C₂-C₃-alkenyl”), it being understood that in the case in which said alkenyl group contains more than one double bond, then said double bonds may be isolated from, or conjugated with, each other.

Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)vinyl, buta-1,3-dienyl, penta-1,4-dienyl, hexa-1,5-dienyl, or methylhexadienyl group. Particularly, said group is vinyl or allyl.

The term “C₂-C₆-alkynyl” is to be understood as preferably meaning a linear or branched, monovalent hydrocarbon group which contains one or more triple bonds, and which contains 2, 3, 4, 5, 6 carbon atoms, particularly 2 or 3 carbon atoms (“C₂-C₃-alkynyl”).

Said C₂-C₁₀-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-inyl, hex-3-inyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-inyl, 1,1-dimethylbut-3-ynyl, 1,1-dimethylbut-2-ynyl, or 3,3-dimethylbut-1-ynyl group. Particularly, said alkynyl group is ethynyl, prop-1-ynyl, or prop-2-ynyl.

The term “C₃-C₁₀-cycloalkyl” is to be understood as preferably meaning a saturated, monovalent, mono-, or bicyclic hydrocarbon ring which contains 3, 4, 5, 6, 7, 8, 9, or 10 carbon atoms, particularly 3, 4, 5, or 6 carbon atoms (“C₃-C₆-cycloalkyl”).

Said C₃-C₁₀-cycloalkyl group is for example, a monocyclic hydrocarbon ring, e.g. a cyclopropyl, cyclobutyl, cyclopentyl, cyclohexyl, cycloheptyl, cyclooctyl, cyclononyl or cyclodecyl group, or a bicyclic hydrocarbon ring, e.g. a perhydropentalenylene or decalin ring. Said cycloalkyl ring can optionally contain one or more double bonds e.g. cycloalkenyl, such as a cyclopropenyl, cyclobutenyl, cyclopentenyl, cyclohexenyl, cycloheptenyl, cyclooctenyl, cyclononenyl, or cyclodecenyl group, wherein the bond between said ring with the rest of the molecule may be to any carbon atom of said ring, be it saturated or unsaturated.

The term “3- to 10-membered heterocycloalkyl” is to be understood as preferably meaning a saturated or partially unsaturated, monovalent, mono- or bicyclic hydrocarbon ring which contains 2, 3, 4, 5, 6, 7, 8, or 9 carbon atoms, and one or more heteroatom-containing groups selected from C(═O), O, S, S(═O), S(═O)₂, NH, NR′, wherein R′ represents a C₁-C₆-alkyl, C₃-C₆-cycloalkyl, C₃-C₆ heterocycloalkyl, C(═O)R⁹, C(═O)NR¹⁰R¹¹, —S(═O)₂R⁹, —S(═O)₂NR¹⁰R¹¹ group as defined supra, it being understood that when said R′ represents a C₃-C₆ heterocycloalkyl group, then said C₃-C₆ heterocycloalkyl group is present only once. Particularly, said ring can contain 2, 3, 4, or 5 carbon atoms, and one or more of the above-mentioned heteroatom-containing groups (a “3- to 6-membered heterocycloalkyl”), more particularly said ring can contain 4 or 5 carbon atoms, and one or more of the above-mentioned heteroatom-containing groups (a “5- to 6-membered heterocycloalkyl”). Non-limiting examples include aziridinyl, azetidinyl, oxetanyl, thietanyl, pyrrolidinyl, pyrazolidinyl, tetrahydrofuranyl, thiolanyl, sulfolanyl, 1,3-dioxolanyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, morpholinyl, thiomorpholinyl, 1,1-dioxidothiomorpholinyl, perhydro-azepinyl, perhydro-1,4-diazepinyl, perhydro-1,4-oxazepinyl, perhydroazocinyl, octahydropyrrolo-[3,4-b]pyrrolyl, octahydroisoindolyl, octahydropyrrolo[3,4-b]pyridyl, octahydropyrrolo[1,2-a]pyrazinyl, decahydroisochinolinyl, 7-azabicyclo[2.2.1]heptyl, 3-azabicyclo[3.2.0]heptyl, 7-azabicyclo-[4.1.0]heptyl, 2,5-diazabicyclo[2.2.1]heptyl, 2-oxa-5-azabicyclo[2.2. I]heptyl, 2-azabicyclo-[2.2.2]octyl, 3-azabicyclo[3.2.1]octyl, 8-azabicyclo[3.2.1]octyl, 8-oxa-3-azabicyclo[3.2.1]octyl, 3-oxa-9-azabicyclo[3.3.1]nonyl. Particular preference is given to 5- to 7-membered monocyclic heterocycloalkyl radicals having up to 2 heteroatoms selected from the group consisting of N, O and S, such as illustratively and preferably tetrahydrofuranyl, 1,3-dioxolanyl, pyrrolidinyl, tetrahydropyranyl, 1,4-dioxanyl, piperidinyl, piperazinyl, morpholinyl, thiomorpholinyl, perhydro-azepinyl, perhydro-1,4-diazepinyl and perhydro-1,4-oxazepinyl.

The term “aryl” is to be understood as preferably meaning a monovalent, aromatic or partially aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group, or a biphenyl group, or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a ring having 13 carbon atoms, (a “C₁₃-aryl” group), e.g. a fluorenyl group, or a ring having 14 carbon atoms, (a “C₁₄-aryl” group), e.g. an anthranyl group.

The term “heteroaryl” is understood as preferably meaning a monovalent, aromatic or partially aromatic, mono- or bicyclic ring system having 5, 6, 7, 8, 9, 10, 11, 12, 13 or 14 ring atoms (a “5- to 14-membered heteroaryl” group), particularly 5 or 6 or 9 or 10 atoms, and which can partially be saturated, and which contains at least one heteroatom which may be identical or different, said heteroatom being such as oxygen, nitrogen or sulfur, and can be monocyclic, bicyclic, or tricyclic, and in addition in each case can be benzocondensed. Preference is given to 6-membered heteroaryl radicals having up to 2 nitrogen atoms, and to 5-membered heteroaryl radicals having up to 3 heteroatoms. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl, tetrazolyl and benzo derivatives thereof, such as, for example, benzofuranyl, benzothienyl, benzoxazolyl, benzisoxazolyl, benzimidazolyl, benzotriazolyl, indazolyl, indolyl, isoindolyl, or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl. More particularly, heteroaryl is selected from thienyl, oxazolyl, thiazolyl, 1H-tetrazol-5-yl, pyridyl, benzothienyl, or furanyl.

The term “alkylene” or “alkylen-” is understood as preferably meaning an optionally substituted hydrocarbon chain (or “tether”) having 1, 2, 3, 4, 5, or 6 carbon atoms, i.e. an optionally substituted —CH₂— (“methylene” or “single membered tether” or, for example —C(Me)₂-),

—CH₂—CH₂— (“ethylene”, “dimethylene”, or “two-membered tether”), —CH₂—CH₂—CH₂— (“propylene”, “trimethylene”, or “three-membered tether”), —CH₂—CH₂—CH₂—CH₂— (“butylene”, “tetramethylene”, or “four-membered tether”), —CH₂—CH₂—CH₂—CH₂—CH₂— (“pentylene”, “pentamethylene” or “five-membered ether”), or —CH₂—CH₂—CH₂—CH₂—CH₂—CH₂— (“hexylene”, “hexamethylene”, or six-membered tether”) group. Particularly, said alkylene tether has 1, 2, 3, 4, or 5 carbon atoms, more particularly 1 or 2 carbon atoms.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, C₁-C₆; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₁₀”, as used throughout this text, e.g. in the context of the definition of “C₃-C₁₀-cycloalkyl”, is to be understood as meaning a cycloalkyl group having a finite number of carbon atoms of 3 to 10, i.e. 3, 4, 5, 6, 7, 8, 9 or 10 carbon atoms, particularly 3, 4, 5 or 6 carbon atoms. It is to be understood further that said term “C₃-C₁₀” is to be interpreted as any sub-range comprised therein, e.g. C₃-C₁₀, C₄-C₉, C₅-C₈, C₆-C₇; particularly C₃-C₆.

Oxo represents a double-bonded oxygen atom.

As used herein, the term “one or more times”, e.g. in the definition of the substituents of the compounds of the general formulae of the present invention, is understood as meaning “one, two, three, four or five times, particularly one, two, three or four times, more particularly one, two or three times, even more particularly one or two times”.

Throughout this document, for the sake of simplicity, the use of singular language is given preference over plural language, but is generally meant to include the plural language if not otherwise stated. E.g., the expression “A method of treating a disease in a patient, comprising administering to a patient an effective amount of a compound of formula (I)” is meant to include the simultaneous treatment of more than one disease as well as the administration of more than one compound of formula (I).

A “*” in a chemical formula indicates a stereogenic center.

Particular forms of embodiment of compounds of the general formula (I) as described above are going to be illustrated in the following.

In conjunction with the above or below definitions and embodiments, compounds according to formula (I), (Ia), (Ib) are in particular those in which R¹ is selected from the group consisting of C₁-C₆-alkyl, C₃-C₁₀-cycloalkyl.

Furthermore, for compounds according to formula (I), (Ia), (Ib) as a particular embodiment according to the invention R² is a phenyl group.

R⁴ within formula (I), (Ia), (Ib) as an embodiment according to the invention is a halogen, a C₁-C₆-alkoxy, C₁-C₆-haloalkoxy, C(O)O—C₁-C₆-alkyl, C(O)OH, or C(O)NH₂ group.

A compound according to formula (I), (Ia), (Ib) of the present invention comprises, according to a further particular embodiment, R² being a phenyl group substituted in para with R⁴ being a fluorine or a OCF₂H.

Another embodiment according to the invention is provided by compounds according to formula (I), (Ia), (Ib) in which R² is a phenyl group substituted in meta with R⁴ being a C₁-C₆-alkoxy, C₁-C₆-haloalkoxy, or C(O)O—C₁-C₆-alkyl.

With reference to particular forms of embodiment of compounds according to formula (I), (Ia), (Ib), the groups R³ and R^{5a} are defined as follows:
R³ is selected from the group consisting of C(O)NH(R^{5a}) and
R^{5a} is C₃-C₁₀-cycloalkyl, C₃-C₁₀-cycloalkyl-C₁-C₆-alkylen-, aryl, aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

According to a further particular alternative compounds according to formula (I), (Ia), (Ib) comprise groups R³ and R⁶ being defined as follows:
R³ is N(H)C(O)R⁶, and
R⁶ is C₃-C₁₀-cycloalkyl, C₃-C₁₀-cycloalkyl-C₁-C₆-alkylen-, aryl, aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

A further form of embodiment according to the invention refers to compounds according to formula (I), (Ia), (Ib) in which R^{5a} is C₃-C₁₀-cycloalkyl, C₃-C₁₀-cycloalkyl-C₁-C₆-alkylen-, aryl or aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

Another alternative according to the invention comprises compounds according to formula (I), (Ia), (Ib) in which R^{5b} is a hydrogen or C₁-C₆-alkyl.

Compounds according to formula (I), (Ia), (Ib) comprise according to a specific form of the invention R⁶ being a C₃-C₁₀-cycloalkyl, C₃-C₁₀-cycloalkyl-C₁-C₆-alkylen-, aryl, aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

According to a further particular form of embodiment of the invention, compounds according to formula (I), (Ia), (Ib) comprise R^{5a}, R⁶ and R⁷ being selected from the group consisting of cyclopropyl, cyclopropyl-CH₂—, cyclopentyl, cyclopentyl-CH₂—, cyclohexyl, cyclohexyl-CH₂—, phenyl, phenyl-CH₂—, pyridyl, pyridyl-CH₂—, 3,4-dihydro-2H-chromen-4-yl, optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(CH₃)₂ or, more particularly, R^{5a}, R⁶ and R⁷ being selected from the group consisting of cyclopropyl, cyclopropyl-CH₂—, cyclopentyl, cyclopentyl-CH₂—, cyclohexyl, cyclohexyl-CH₂—, 3,4-dihydro-2H-chromen-4-yl; and phenyl, phenyl-CH₂—, pyridyl, pyridyl-CH₂—, substituted one or two times with a fluorine, chlorine, hydroxy, CH₃, CF, CF₂H, C₁-C₆-alkoxy, C₁-C₆-haloalkoxy, C(O)OH, C(O)OCH₃, CN, C(O)NH₂, S(O)₂—CH₃, S(O)₂NH₂, S(O)₂N(CH₃)₂.

Furthermore forms of embodiments according to the present invention comprise in particular compounds according to formula (Ia) in which
x is 1
R¹ is selected from the group consisting of methyl, ethyl, cyclopropyl, ethinyl and allyl;
R⁴ is a fluorine, C₁-C₆-alkoxy, C₁-C₆-haloalkoxy, C(O)OC₁-C₆-alkyl.

Particular embodiments of the invention refer to a compound according to formula (Ia) being defined by x equal to 2, R¹ is selected from the group consisting of methyl, ethyl, cyclopropyl, ethinyl and allyl; R⁴ is a fluorine, C₁-C₆-alkoxy, C₁-C₆-haloalkoxy, C(O)O—C₁-C₆-alkyl.

A compound according to formula (Ia) is defined in accordance to a specific form of embodiment of the invention by R⁴ being in the para or meta position on the phenyl radical of formula (Ia), in particular R⁴ is a fluorine or OCF₂H in the para position on the phenyl radical of formula (Ia), or as further particular alternative by R⁴ being C₁-C₆-alkoxy or C(O)O—C₁-C₆-alkyl in meta position on the phenyl radical of formula (Ia).

According to a further particular form of embodiment of the invention, compounds according to formula (Ia) comprise R^{5a} being C₃-C₁₀-cycloalkyl, C₃-C₁₀-cycloalkyl-C₁-C₆-alkylen-, aryl or aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

More particularly compounds according to formula (Ia) comprise R^{5a} being a cyclopropyl, cyclopropyl-CH₂—, cyclopentyl, cyclopentyl-CH₂—, cyclohexyl, cyclohexyl-CH₂—, phenyl, phenyl-CH₂—, pyridyl, pyridyl-CH₂—, 3,4-dihydro-2H-chromen-4-yl, optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(CH₃)₂.

Furthermore particular forms of embodiments of compounds according to formula (Ib) are those in which R¹ is C₁-C₆-alkyl and R⁴ is a fluorine, C₁-C₆-alkoxy, C₁-C₆-haloalkoxy, C(O)OC₁-C₆-alkyl.

Compounds according to formula (Ib) comprise in particular R⁴ in the para or meta position on the phenyl radical of formula (Ib).

Particular embodiments of the invention refer to a compound according to formula (Ib) in which R¹ is a methyl, ethyl, cyclopropyl, ethinyl and allyl and R⁴ is a fluorine, C₁-C₆-alkoxy, C₁-C₆-haloalkoxy, C(O)OC₁-C₆-alkyl, or in a further specific alternative R¹ is a methyl and R⁴ is a fluorine in the para position at the phenyl radical of formula (Ib).

According to a further particular form of embodiment of the invention, compounds according to formula (Ib) comprise R^{5a} being aryl or aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

More particularly compounds according to formula (Ib) comprise R^{5a} being a phenyl, phenyl-CH₂—, pyridyl, pyridyl-CH₂—, optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(CH₃)₂.

Compounds according to the invention are:
N-[(3-Chloropyridin-2-yl)methyl]-1-[(4-fluorophenyl)sulfonyl]-2-methyl-1,2,2′,3′,5′,6′-hexahydrospiro[indole-3,4′-pyran]-5-carboxamide
N-(2-chlorobenzyl)-1-[(4-fluorophenyl)sulfonyl]-2-methyl-1,2,2′,3′,5′,6′-hexahydrospiro[indole-3,4′-pyran]-5-carboxamide
1-[(4-fluorophenyl)sulfonyl]-2-methyl-N-{[3-(trifluoromethyl)pyridin-2-yl]methyl}-1,2,2′,3′,5′,6′-hexahydrospiro[indole-3,4′-pyran]-5-carboxamide
1-[(4-fluorophenyl)sulfonyl]-2-methyl-N-[2-(trifluoromethyl)benzyl]-1,2,2′,3′,5′,6′-hexahydrospiro[indole-3,4′-pyran]-5-carboxamide
N-[(3-chloro-5-fluoropyridin-2-yl)methyl]-1-[(4-fluorophenyl)sulfonyl]-2-methyl-1,2,2′,3′,5′,6′-hexahydrospiro[indole-3,4′-pyran]-5-carboxamide
1-[(4-fluorophenyl)sulfonyl]-2-methyl-N-(2-pyridylmethyl)-1,2,2′,3′,5′,6′-hexahydrospiro[indole-3,4′-pyran]-5-carboxamide
N-(4-fluorobenzyl)-1-[(4-fluorophenyl)sulfonyl]-2-methyl-1,2,2′,3′,5′,6′-hexahydrospiro[indole-3,4′-pyran]-5-carboxamide
N-(2-cyanobenzyl)-1-[(4-fluorophenyl)sulfonyl]-2-methyl-1,2,2′,3′,5′,6′-hexahydrospiro[indole-3,4′-pyran]-5-carboxamide
1-[(4-fluorophenyl)sulfonyl]-N-(2-mesylbenzyl)-2-methyl-1,2,2′,3′,5′,6′-hexahydrospiro[indole-3,4′-pyran]-5-carboxamide
1-[(4-fluorophenyl)sulfonyl]-N-(3-mesylphenyl)-2-methyl-1,2,2′,3′,5′,6′-hexahydrospiro[indole-3,4′-pyran]-5-carboxamide
N-[3-(N,N-dimethylsulfamoyl)phenyl]-1-[(4-fluorophenyl)sulfonyl]-2-methyl-1,2,2′,3′,5′,6′-hexahydrospiro[indole-3,4′-pyran]-5-carboxamide
N-(2-chlorobenzyl)-2-cyclopropyl-1-[(4-fluorophenyl)sulfonyl]-1,2,2′,3′,5′,6′-hexahydrospiro[indole-3,4′-thiopyran]-5-carboxamide
N-(2-chlorobenzyl)-2-cyclopropyl-1-[(4-fluorophenyl)sulfonyl]-1,2,2′,3′,5′,6′-hexahydrospiro[indole-3,4′-thiopyran]-5-carboxamide 1′-oxide
N-[(3-chloropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide
N-(2-chloro-4-fluorobenzyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
N-[(3-chloro-5-fluoropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
N-(2-chlorobenzyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
N-(2-chloro-4-fluoro-α,α-dimethylbenzyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-N-(4-fluoro-α,α-dimethylbenzyl)-1-[(4-fluorophenyl)sulfonyl]-1,2,2′,3′,5′,6′-hexahydrospiro[indole-3,4′-thiopyran]-5-carboxamide 1′,1′-dioxide
N-[1-(2-chlorophenyl)cyclopropyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-(2-pyridylmethyl)-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-(3-mesylphenyl)-1,2,2′,3′,5′,6′-hexahydrospiro[indole-3,4′-thiopyran]-5-carboxamide 1′,1′-dioxide
N-(3-chlorophenyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
N-[2-(2-chlorophenyl)ethyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
N-[(3-chloropyridin-2-yl)methyl]-1-[(4-fluorophenyl)sulfonyl]-2-methyl-1,2,2′,3′,5′,6′-hexahydrospiro[indole-3,4′-thiopyran]-5-carboxamide 1′,1′-dioxide
N-(2-chloro-4-fluoro-α,α-dimethylbenzyl)-1-[(4-fluorophenyl)sulfonyl]-2-methyl-1,2,2′,3′,5′,6′-hexahydrospiro[indole-3,4′-thiopyran]-5-carboxamide 1′,1′-dioxide
N-[(3-chloro-5-fluoropyridin-2-yl)methyl]-1-[(4-fluorophenyl)sulfonyl]-2-methyl-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-(5-methylpyridin-2-yl)-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-(3-sulfamoylphenyl)-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-[(3-methylpyridin-2-yl)methyl]-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-[2-(trifluoromethyl)benzyl]-1,2,2′,3′,5′,6′-hexahydrospiro[indole-3,4′-thiopyran]-5-carboxamide 1′,1′-dioxide
2-cyclopropyl-N-(3,4-dihydro-2H-chromen-4-yl)-1-[(4-fluorophenyl)sulfonyl]-1,2,2′,3′,5′,6′-hexahydrospiro[indole-3,4′-thiopyran]-5-carboxamide 1′,1′-dioxide
methyl 3-[({2-cyclopropyl-1-[(4-fluorophenyl)sulfonyl]-1′,1′-dioxido-1,2,2′,3′,5′,6′-hexahydrospiro[indole-3,4′-thiopyran]-5-yl}carbonyl)amino]benzoate
2-cyclopropyl-N-(cyclopropylmethyl)-1-[(4-fluorophenyl)sulfonyl]-1,2,2′,3′,5′,6′-hexahydrospiro[indole-3,4′-thiopyran]-5-carboxamide 1′,1′-dioxide
N-(cyclohexylmethyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-N-[3-(dimethylsulfamoyl)phenyl]-1-[(4-fluorophenyl)sulfonyl]-1,2,2′,3′,5′,6′-hexahydrospiro[indole-3,4′-thiopyran]-5-carboxamide 1′,1′-dioxide
N-(cyclopentylmethyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-1-[(3-methoxyphenyl)sulfonyl]-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide
2-cyclopropyl-1-[(3-methoxyphenyl)sulfonyl]-N-[2-(trifluoromethyl)benzyl]-1,2,2′,3′,5′,6′-hexahydrospiro[indole-3,4′-thiopyran]-5-carboxamide 1′,1′-dioxide
N-[(3-chloropyridin-2-yl)methyl]-2-cyclopropyl-1-[(3-methoxyphenyl)sulfonyl]-1,2,2′,3′,5′,6′-hexahydrospiro[indole-3,4′-thiopyran]-5-carboxamide 1′,1′-dioxide
2-cyclopropyl-1-[(3-methoxyphenyl)sulfonyl]-N-(3-sulfamoylphenyl)-1,2,2′,3′,5′,6′-hexahydrospiro[indole-3,4′-thiopyran]-5-carboxamide 1′,1′-dioxide
2-cyclopropyl-N-[3-(dimethylsulfamoyl)phenyl]-1-[(3-methoxyphenyl)sulfonyl]-1,2,2′,3′,5′,6′-hexahydrospiro[indole-3,4′-thiopyran]-5-carboxamide 1′,1′-dioxide
N-(2-chlorobenzyl)-2-cyclopropyl-1-[(3-methoxyphenyl)sulfonyl]-1,2,2′,3′,5′,6′-hexahydrospiro[indole-3,4′-thiopyran]-5-carboxamide 1′,1′-dioxide
2-cyclopropyl-1-[(3-methoxyphenyl)sulfonyl]-N-[(3-methylpyridin-2-yl)methyl]-1,2,2′,3′,5′,6′-hexahydrospiro[indole-3,4′-thiopyran]-5-carboxamide 1′,1′-dioxide
N-(2-chloro-4-fluorobenzyl)-2-cyclopropyl-1-[(3-methoxyphenyl)sulfonyl]-1,2,2′,3′,5′,6′-hexahydrospiro[indole-3,4′-thiopyran]-5-carboxamide 1′,1′-dioxide
2-cyclopropyl-N-(2-fluorobenzyl)-1-[(3-methoxyphenyl)sulfonyl]-1,2,2′,3′,5′,6′-hexahydrospiro[indole-3,4′-thiopyran]-5-carboxamide 1′,1′-dioxide
methyl 3-[({2-cyclopropyl-1-[(3-methoxyphenyl)sulfonyl]-1′,1′-dioxido-1,2,2′,3′,5′,6′-hexahydrospiro[indole-3,4′-thiopyran]-5-yl}carbonyl)amino]benzoate
3-[({2-cyclopropyl-1-[(4-fluorophenyl)sulfonyl]-1′,1′-dioxido-1,2,2′,3′,5′,6′-hexahydrospiro[indole-3,4′-thiopyran]-5-yl}carbonyl)amino]benzoic acid
3-[({2-cyclopropyl-1-[(3-methoxyphenyl)sulfonyl]-1′,1′-dioxido-1,2,2′,3′,5′,6′-hexahydrospiro[indole-3,4′-thiopyran]-5-yl}carbonyl)amino]benzoic acid
N-(3-carbamoylphenyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-[(3-fluoropyridin-2-yl)methyl]-1,2,2′,3′,5′,6′-hexahydrospiro[indole-3,4′-thiopyran]-5-carboxamide 1′,1′-dioxide
2-cyclopropyl-N-[(3-fluoropyridin-2-yl)methyl]-1-[(3-methoxyphenyl)sulfonyl]-1,2,2′,3′,5′,6′-hexahydrospiro[indole-3,4′-thiopyran]-5-carboxamide 1′,1′-dioxide
methyl 3-({5-[N-(2-chlorobenzyl)carbamoyl]-1′,1′-dioxido-2-(prop-2-en-1-yl)-1,2,2′,3′,5′,6′-hexahydrospiro[indole-3,4′-thiopyran]-1-yl}sulfonyl)benzoate
methyl 3-({5-[N-(2-chlorobenzyl)carbamoyl]-1′,1′-dioxido-2-vinyl-1,2,2′,3′,5′,6′-hexahydrospiro[indole-3,4′-thiopyran]-1-yl}sulfonyl)benzoate
3-({5-[(2-chlorobenzyl)carbamoyl]-1′,1′-dioxido-2-(prop-2-en-1-yl)-2′,3′,5′,6′-tetrahydrospiro[indole-3,4′-thiopyran]-1 (2H)-yl}sulfonyl)benzoic acid
N-[(3-chloropyridin-2-yl)methyl]-1-[(4-fluorophenyl)sulfonyl]-2-(prop-2-en-1-yl)-1,2,2′,3′,5′,6′-hexahydrospiro[indole-3,4′-thiopyran]-5-carboxamide 1′,1′-dioxide
methyl 3-[({1-[(4-fluorophenyl)sulfonyl]-1′,1′-dioxido-2-(prop-2-en-1-yl)-1,2,2′,3′,5′,6′-hexahydrospiro[indole-3,4′-thiopyran]-5-yl}carbonyl)amino]benzoate
3-[({1-[(4-fluorophenyl)sulfonyl]-1′,1′-dioxido-2-(prop-2-en-1-yl)-1,2,2′,3′,5′,6′-hexahydrospiro[indole-3,4′-thiopyran]-5-yl}carbonyl)amino]benzoic acid
methyl 3-({5-[(2-chlorobenzyl)carbamoyl]-2-cyclopropyl-1′,1′-dioxido-2′,3′,5′,6′-tetrahydrospiro[indole-3,4′-thiopyran]-1 (2H)-yl}sulfonyl)benzoate
3-({5-[(2-chlorobenzyl)carbamoyl]-2-cyclopropyl-1′,1′-dioxido-2′,3′,5′,6′-tetrahydrospiro[indole-3,4′-thiopyran]-1(2H)-yl}sulfonyl)benzoic acid
N-(3-{[bis(dimethylamino)methylidene]sulfamoyl}phenyl)-2-cyclopropyl-1-[(3-methoxyphenyl)sulfonyl]-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-(1,2-oxazol-3-yl)-1,2,2′,3′,5′,6′-hexahydrospiro[indole-3,4′-thiopyran]-5-carboxamide 1′,1′-dioxide
N-(3-{[bis(dimethylamino)methylidene]sulfamoyl}phenyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-{[5-(trifluoromethyl)pyridin-2-yl]methyl}-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-{3-[(5-methyl-1,2-oxazol-3-yl)sulfamoyl]phenyl}-1,2,2′,3′,5′,6′-hexahydrospiro[indole-3,4′-thiopyran]-5-carboxamide 1′,1′-dioxide
N-(2-chlorophenyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-N-[2-(difluoromethyl)benzyl]-1-[(4-fluorophenyl)sulfonyl]-1,2,2′,3′,5′,6′-hexahydrospiro[indole-3,4′-thiopyran]-5-carboxamide 1′,1′-dioxide
2-cyclopropyl-1-[(4-fluorophenyl)sulfonyl]-N-(2-hydroxybenzyl)-1,2,2′,3′,5′,6′-hexahydrospiro[indole-3,4′-thiopyran]-5-carboxamide 1′,1′-dioxide
N-[(3-chloropyridin-2-yl)methyl]-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide
N-(5-chloropyridin-3-yl)-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide
1-[(4-cyanophenyl)sulfonyl]-2-cyclopropyl-N-[2-(trifuoromethyl)benzyl]-1,2,2′,3′,5′,6′-hexahydrospiro[indole-3,4′-thiopyran]-5-carboxamide 1′,1′-dioxide
1-[(4-cyanophenyl)sulfonyl]-2-cyclopropyl-N-(1,3-oxazol-2-yl)-1,2,2′,3′,5′,6′-hexahydrospiro[indole-3,4′-thiopyran]-5-carboxamide 1′,1′-dioxide
N-(2-chlorophenyl)-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide
1-[(4-cyanophenyl)sulfonyl]-2-cyclopropyl-N-(2-fluorophenyl)-1,2,2′,3′,5′,6′-hexahydrospiro[indole-3,4′-thiopyran]-5-carboxamide 1′,1′-dioxide
N-(2-chlorobenzyl)-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide
N-(5-chloropyridin-3-yl)-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide
1-[(3-cyanophenyl)sulfonyl]-2-cyclopropyl-N-(1,3-oxazol-2-yl)-1,2,2′,3′,5′,6′-hexahydrospiro[indole-3,4′-thiopyran]-5-carboxamide 1′,1′-dioxide
1-[(3-cyanophenyl)sulfonyl]-2-cyclopropyl-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide
1-[(3-cyanophenyl)sulfonyl]-2-cyclopropyl-N-(1,2-oxazol-3-yl)-1,2,2′,3′,5′,6′-hexahydrospiro[indole-3,4′-thiopyran]-5-carboxamide 1′,1′-dioxide
N-(2-chlorobenzyl)-2-cyclopropyl-1-{[3-(trifluoromethoxy)phenyl]sulfonyl}-1,2,2′,3′,5′,6′-hexahydrospiro[indole-3,4′-thiopyran]-5-carboxamide 1′,1′-dioxide
methyl 3-{[(2-cyclopropyl-1′,1′-dioxido-1-{[3-(trifluoromethoxy)phenyl]sulfonyl}-1,2,2′,3′,5′,6′-hexahydrospiro[indole-3,4′-thiopyran]-5-yl)carbonyl]amino}benzoate
3-{[(2-cyclopropyl-1′,1′-dioxido-1-{[3-(trifluoromethoxy)phenyl]sulfonyl}-1,2,2′,3′,5′,6′-hexahydrospiro[indole-3,4′-thiopyran]-5-yl)carbonyl]amino}benzoic acid
2-cyclopropyl-1-{[3-(trifluoromethoxy)phenyl]sulfonyl}-N-{[3-(trifuoromethyl)pyridin-2-yl]methyl}-1,2,2′,3′,5′,6′-hexahydrospiro[indole-3,4′-thiopyran]-5-carboxamide 1′,1′-dioxide
2-cyclopropyl-N-(5-methylpyridin-3-yl)-1-{[3-(trifluoromethoxy)phenyl]sulfonyl}-1,2,2′,3′,5′,6′-hexahydrospiro[indole-3,4′-thiopyran]-5-carboxamide 1′,1′-dioxide
N-(2-chlorobenzyl)-2-cyclopropyl-1-{[3-(difluoromethoxy)phenyl]sulfonyl}-1,2,2′,3′,5′,6′-hexahydrospiro[indole-3,4′-thiopyran]-5-carboxamide 1′,1′-dioxide
2-cyclopropyl-1-{[3-(difluoromethoxy)phenyl]sulfonyl}-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide
N-(5-chloropyridin-3-yl)-2-cyclopropyl-1-{[3-(difluoromethoxy)phenyl]sulfonyl}-1,2,2′,3′,5′,6′-hexahydrospiro[indole-3,4′-thiopyran]-5-carboxamide 1′,1′-dioxide
methyl 3-{[(2-cyclopropyl-1-{[3-(difluoromethoxy)phenyl]sulfonyl}-1′,1′-dioxido-1,2,2′,3′,5′,6′-hexahydrospiro[indole-3,4′-thiopyran]-5-yl)carbonyl]amino}benzoate
3-{[(2-cyclopropyl-1-{[3-(difluoromethoxy)phenyl]sulfonyl}-1′,1′-dioxido-1,2,2′,3′,5′,6′-hexahydrospiro[indole-3,4′-thiopyran]-5-yl)carbonyl]amino}benzoic acid
2-cyclopropyl-1-{[4-(difluoromethoxy)phenyl]sulfonyl}-N-[2-(difluoromethyl)benzyl]-1,2,2′,3′,5′,6′-hexahydrospiro[indole-3,4′-thiopyran]-5-carboxamide 1′,1′-dioxide
2-cyclopropyl-1-{[4-(difluoromethoxy)phenyl]sulfonyl}-N-[2-(trifluoromethyl)benzyl]-1,2,2′,3′,5′,6′-hexahydrospiro[indole-3,4′-thiopyran]-5-carboxamide 1′,1′-dioxide
2-cyclopropyl-1-{[4-(difluoromethoxy)phenyl]sulfonyl}-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide
1-[(4-carbamoylphenyl)sulfonyl]-N-(2-chlorobenzyl)-2-cyclopropyl-1,2,2′,3′,5′,6′-hexahydrospiro[indole-3,4′-thiopyran]-5-carboxamide 1′,1′-dioxide
1-[(4-carbamoylphenyl)sulfonyl]-2-cyclopropyl-N-{3-[(1-methylpyrrolidin-2-ylidene)sulfamoyl]phenyl}-1,2,2′,3′,5′,6′-hexahydrospiro[indole-3,4′-thiopyran]-5-carboxamide 1′,1′-dioxide
1-[(4-carbamoylphenyl)sulfonyl]-2-cyclopropyl-N-[3-(1,3-thiazol-2-ylsulfamoyl)phenyl]-1,2,2′,3′,5′,6′-hexahydrospiro[indole-3,4′-thiopyran]-5-carboxamide 1′,1′-dioxide
N-{2-cyclopropyl-1-[(4-fluorophenyl)sulfonyl]-1′,1′-dioxido-1,2,2′,3′,5′,6′-hexahydrospiro[indole-3,4′-thiopyran]-5-yl}cyclopropanecarboxamide
N-{2-cyclopropyl-1-[(4-fluorophenyl)sulfonyl]-1′,1′-dioxido-1,2,2′,3′,5′,6′-hexahydrospiro[indole-3,4′-thiopyran]-5-yl}cyclohexanecarboxamide
N-{2-cyclopropyl-1-[(4-fluorophenyl)sulfonyl]-1′,1′-dioxido-1,2,2′,3′,5′,6′-hexahydrospiro[indole-3,4′-thiopyran]-5-yl}cyclopentanecarboxamide

Another embodiment of the present invention provides compounds according to general formula (I), (Ia), (Ib) and related specific embodiments for use as a medicament.

In another embodiment, the present invention provides a method of treating GnRH related disorder in a patient in need of such treatment, comprising administering to the patient an effective amount of a compound according to the invention as defined above.

In still another aspect, the invention provides use of a compound according to the invention as defined above for manufacturing a pharmaceutical composition for the treatment or prevention of GnRH related disorders.

The term “treating” or “treatment” as stated throughout this document is used conventionally, e.g., the management or care of a subject for the purpose of combating, alleviating, reducing, relieving, improving the condition of a disease or disorder, such as for example endometriosis and uterine fibroids.

The term “subject” or “patient” includes organisms which are capable of suffering from a disorder or who could otherwise benefit from the administration of a compound of the invention, such as human and non-human animals. Preferred humans include human patients suffering from or prone to suffering from disorders, such as for example endometriosis and uterine fibroids. The term “non-human animals” includes vertebrates, e.g., mammals, such as non-human primates, sheep, cows, dogs, cats and rodents, e.g., mice, and non-mammals, such as chickens, amphibians, reptiles, etc.

In another aspect, the invention provides a pharmaceutical composition comprising a compound according to the invention, together with a pharmaceutically acceptable carrier. In still another aspect, the invention provides a process for preparing a pharmaceutical composition. The process includes the step of combining at least one compound according to the invention as defined above with at least one pharmaceutically acceptable carrier, and bringing the resulting combination into a suitable administration form.

The compounds according to general formula (I), (Ia), (Ib) are used as a medicament. In particular, said compounds are used to treat sexual hormone-related conditions in both men and women, as well as a mammal in general (also referred to herein as a “subject”). For example, such conditions include endometriosis, uterine fibroids, polycystic ovarian disease, hirsutism, precocious puberty, gonadal steroid-dependent neoplasia such as cancers of the prostate, breast and ovary, gonadotrope pituitary adenomas, sleep apnea, irritable bowel syndrome, premenstrual syndrome, benign prostatic hypertrophy, and infertility (e.g., assisted reproductive therapy such as in vitro fertilization).

The compounds according to general formula (I), (Ia), (Ib) are further used as contraceptive. The compounds of this invention are also useful as an adjunct to treatment of growth hormone deficiency and short stature, and for the treatment of systemic lupus erythematosus.

According to a further embodiment of the present invention the compounds according to general formula (I), (Ia), (Ib) are also useful and can be used in combination with androgens, estrogens, progestins, SERMs, antiestrogens and antiprogestins for the treatment of endometriosis, uterine fibroids, and in contraception, as well as in combination with an angiotensin-converting enzyme inhibitor, an angiotensin II-receptor antagonist, or a renin inhibitor for the treatment of uterine fibroids.

A combination of compounds according to general formula (I), (Ia), (Ib) with bisphosphonates and other agents for the treatment and/or prevention of disturbances of calcium, phosphate and bone metabolism, and in combination with estrogens, SERMs, progestins and/or androgens for the prevention or treatment of bone loss or hypogonadal symptoms such as hot flushes during therapy with a GnRH antagonist is also part of the present invention.

The methods of this invention include administering an effective amount of a GnRH receptor antagonist, preferably in the form of a pharmaceutical composition, to a mammal in need thereof. Thus, in still a further embodiment, pharmaceutical compositions are disclosed containing one or more GnRH receptor antagonists of this invention in combination with a pharmaceutically acceptable carrier and/or diluent.

These and other aspects of the invention will be apparent upon reference to the following detailed description. To this end, various references are set forth herein which describe in more detail certain background information, procedures, compounds and/or compositions, and are each hereby incorporated by reference in their entirety.

The compounds of the present invention may generally be utilized as the free acid or free base. Alternatively, the compounds of this invention may be used in the form of acid or base addition salts.

Thus, the term “pharmaceutically acceptable salt” of compounds of general formula (I), (Ia), (Ib) is intended to encompass any and all acceptable salt forms.

In addition, prodrugs are also included within the context of this invention. Prodrugs are any covalently bonded carriers that release a compound of general formula (I), (Ia), (Ib) in vivo when such prodrug is administered to a patient. Prodrugs are generally prepared by modifying functional groups in a way such that the modification is cleaved, either by routine manipulation or in vivo, yielding the parent compound.

Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of general formula (I), (Ia), (Ib). Further, in the case of a carboxylic acid (—COOH), esters may be employed, such as methyl esters, ethyl esters, and the like.

With regard to stereoisomers, the compounds of general formula (I), (Ia), (Ib) may have chiral centers and may occur as racemates, racemic mixtures and as individual enantiomers or diastereomers. All such isomeric forms are included within the present invention, including mixtures thereof. Furthermore, some of the crystalline forms of the compounds of general formula (I), (Ia), (Ib) may exist as polymorphs, which are included in the present invention. In addition, some of the compounds of general formula (I), (Ia), (Ib) may also form solvates with water or other organic solvents. Such solvates are similarly included within the scope of this invention.

The effectiveness of a compound as a GnRH receptor antagonist may be determined by various assay techniques. Assay techniques well known in the field include the use of cultured pituitary cells for measuring GnRH activity (Vale et al., Endocrinology 1972, 91, 562-572) and the measurement of radioligand binding to rat pituitary membranes (Perrin et al., Mol. Pharmacol. 1983, 23, 44-51) or to membranes from cells expressing cloned receptors as described below. Other assay techniques include (but are not limited to) measurement of the effects of GnRH receptor antagonists on the inhibition of GnRH-stimulated calcium flux, modulation of phosphoinositol hydrolysis, and the circulating concentrations of gonadotropins in the castrate animal. Descriptions of these techniques, the synthesis of radiolabeled ligand, the employment of radiolabeled ligand in radioimmunoassay, and the measurement of the effectiveness of a compound as a GnRH receptor antagonist follow.

In another embodiment of the invention, pharmaceutical compositions containing one or more GnRH receptor antagonists are disclosed. For the purposes of administration, the compounds of the present invention may be formulated as pharmaceutical compositions.

Pharmaceutical compositions of the present invention comprise a GnRH receptor antagonist of the present invention and a pharmaceutically acceptable carrier and/or diluent. The GnRH receptor antagonist is present in the composition in an amount which is effective to treat a particular disorder that is, in an amount sufficient to achieve GnRH receptor antagonist activity, and preferably with acceptable toxicity to the patient. Typically, the pharmaceutical compositions of the present invention may include a GnRH receptor antagonist in an amount from 0.1 mg to 500 mg per day dosage depending upon the route of administration, and more typically from 0.5 mg to 150 mg per day. Appropriate concentrations and dosages can be readily determined by one skilled in the art.

Determination of a therapeutically effective amount or a prophylactically effective amount of the compounds of the invention can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective amount or dose, and the prophylactically effective amount or dose, a number of factors are considered by the attending clinician, including, but not limited to: the specific GnRH mediated disorder involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other coadministered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a GnRH receptor antagonist, diluents, dispersing and surface active agents, binders, and lubricants. One skilled in this art may further formulate the GnRH receptor antagonist in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating sex-hormone-related conditions as discussed above. Such methods include administering of a compound of the present invention to a warm-blooded animal in an amount sufficient to treat the condition. In this context, “treat” includes prophylactic administration. Such methods include systemic administration of a GnRH receptor antagonist of this invention, preferably in the form of a pharmaceutical composition as discussed above. As used herein, systemic administration includes oral and parenteral methods of administration. For oral administration, suitable pharmaceutical compositions of GnRH receptor antagonists include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parenteral administration, the compounds of the present invention can be prepared in aqueous injection solutions which may contain, in addition to the GnRH receptor antagonist, buffers, antioxidants, bacteriostats, and other additives commonly employed in such solutions.

### MODE(S) FOR CARRYING OUT THE INVENTION

The following examples are provided for purposes of illustration, not limitation. In summary, the GnRH receptor antagonists of this invention may be assayed by the general methods disclosed above, while the following examples disclose the synthesis of representative compounds of this invention.

### EXPERIMENTAL DETAILS AND GENERAL PROCESSES

The following table lists the abbreviations used in this paragraph and in the examples section as far as they are not explained within the text body.

| Abbreviation | Meaning |
|---|---|
| Ac | acetyl |
| aq. | aqueous |
| BOC | tert-butyloxycarbonyl |
| br. s. | broad singlet |
| d | doublet |
| dbr | broad doublet |
| dd | doublet of doublets |
| ddbr | broad doublet of doublets |
| ddd | doublet of doublet of doublets |
| dt | doublet of triplets |
| DCM | dichloromethane |
| DIPEA | N,N-diisopropylethylamine |
| DMF | N,N-dimethylformamide |
| DMSO | dimethyl sulfoxide |
| eq. | equivalent(s) |
| ESI | electrospray ionization |
| GP | general procedure |
| HATU | 2-(7-aza-1H-benzotriazole-1-yl)-1,1,3,3-tetramethyluronium |
| | hexafluorophosphate) |
| HOAt | 1-hydroxy-7-azabenzotriazole |
| HPLC | high performance liquid chromatography |
| LCMS | liquid chromatography mass spectrometry |
| LDA | lithium diisopropylamide |
| m | multiplet |
| mc | centred multiplet |
| mCPBA | meta-chloroperoxybenzoic acid |
| MS | mass spectrometry |
| NMR | nuclear magnetic resonance spectroscopy: chemical |
| | shifts (δ) are given in ppm |
| q | quartet |
| qbr | broad quartet |
| R_{T} | retention time |
| r.t. or rt or | room temperature |
| room temp. | |
| s | singlet |
| sat. | saturated |
| t | triplet |
| tbr | broad triplet |
| TBAF | tetrabutylammonium fluoride |
| TEA | triethylamine |
| TLC | thin layer chromatography |
| TFA | trifluoroacetic acid |
| THF | tetrahydrofuran |
| UPLC | ultra performance liquid chromatography |
| UPLC-MS | ultra performance liquid chromatography - mass |
| | spectrometry |

NMR peak forms are stated as they appear in the spectra, possible higher order effects have not been considered. Chemical shifts are given in ppm; all spectra were calibrated to solvent residual peak. Integrals are given in integers.

Ultra performance liquid chromatography/liquid chromatography mass spectrometry-methods:

The terms “UPLC-MS (ESI+)” or “UPLC-MS (ESI−)” refer to the following conditions:
Instrument: Waters Acquity UPLC-MS SQD 3001; column: Acquity UPLC BEH C18 1.7 50×2.1 mm; eluent A: water+0.1% vol. formic acid (99%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD; or
Instrument: Waters Acquity UPLC-MS SQD 3001; column: Acquity UPLC BEH C18 1.7 50×2.1 mm; eluent A: water+0.05% vol. formic acid (98%), eluent B: acetonitrile+0.05% vol. formic acid (98%); gradient: 0-1.6 min 1-99% B, 1.6-2.0 min 99% B; flow 0.8 mL/min; temperature: 60° C.; injection: 2 μl; DAD scan: 210-400 nm; ELSD; or
Instrument: Waters Acquity UPLC-MS SQD 3001; column: Acquity UPLC BEH C18 1.7 50×2.1 mm; Eluent A: water+0.2% vol. ammonia (32%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

Analytical characterization of enantiomers was performed by analytical chiral HPLC. In the description of the individual examples is referred to the applied HPLC procedure from the following list:

Method A: Waters: Alliance 2695, DAD 996, ESA: Corona; Flow: 1.0 mL/min; Temperature: 25° C.; Injection: 5.0 μl, 1.0 mg/mL ethanol/methanol 1:1. Columns, solvent system and detection system are specified at the respective example.
Method B1: Dionex: Pump 680, ASI 100, Waters: UV-Detektor 2487; Flow: 1.0 mL/min; Temperature: 25° C.; Injection: 5.0 μl, 1.0 mg/mL ethanol/methanol 1:1; Detection: DAD 280 nm. Columns and solvent systems are specified at the respective example.
Method B2: Dionex: Pump 680, ASI 100, Knauer: UV-Detektor K-2501; Flow: 1.0 mL/min; Temperature: 25° C.; Injection: 5.0 μl, 1.0 mg/mL ethanol/methanol 2:1. Columns, solvent system and detection are specified at the respective example.
Method B3: Dionex: Pump 680, ASI 100, UVD 170U; Flow: 1.0 mL/min; Temperature: rt; Injection: 5.0 μl, 1 mg/mL ethanol; Detection: UV 254 nm. Columns and solvent systems are specified at the respective example.
Method C: Agilent: 1260 AS, MWD, Aurora SFC-module; Flow: 4.0 mL/min; Pressure (outlet): 100 or 120 bar; Temperature: 37.5° C.; Injection: 10.0 μl, 1.0 mg/mL ethanol/methanol 1:1. Columns, solvent system and detection system are specified at the respective example.

Chemical names were generated according to the IUPAC rules [ACD/Name Batch ver. 12.00] or using AutoNom2000 as implemented in MDL ISIS Draw [MDL Information Systems Inc. (Elsevier MDL)]. In some cases generally accepted names of commercially available reagents were used in place of IUPAC names or AutoNom2000 generated names. Stereodescriptors are used according to Chemical Abstracts.

Reactions employing microwave irradiation may be run with a Biotage Initiator® microwave oven optionally equipped with a robotic unit. The reported reaction times employing microwave heating are intended to be understood as fixed reaction times after reaching the indicated reaction temperature.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

The following schemes and general procedures illustrate general synthetic routes to the compounds of general formula (I) of the invention and are not intended to be limiting. It is obvious to the person skilled in the art that the order of transformations as exemplified in Schemes 1 to 6 can be modified in various ways. The order of transformations exemplified in Schemes 1 to 6 is therefore not intended to be limiting. In addition, interconversion of substituents, for example of residues R¹, R², R³, R^{5a}, R^{5b} and R⁶ can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts, Protective Groups in Organic Synthesis, 3rd edition, Wiley 1999).

Compounds of general formula 6 may be synthesized according to the procedures depicted in Scheme 1 from suitably functionalized carboxylic acids of formula 8 by reaction with appropriate amines HN(R^{5a})(R^{5b}) (9). For amide formation, however, all processes that are known from peptide chemistry to the person skilled in the art may be applied. The acids of general formula 8 can be reacted with an appropriate amine in aprotic polar solvents, such as for example DMF, acetonitrile or N-methylpyrrolid-2-one via an activated acid derivative, which is obtainable for example with hydroxybenzotriazole and a carbodiimide such as for example diisopropylcarbodiimide, or else with preformed reagents, such as for example O-(7-azabenzotriazol-1-yl)-1,1,3,3-tetramethyluronium hexafluorophosphate (see for example Chem. Comm. 1994, 201-203), or else with activating agents such as dicyclohexylcarbodiimide/N,N-dimethylaminopyridine or N-ethyl-N′,N′-dimethylaminopropylcarbodiimide/N,N-dimethylaminopyridine. The addition of a suitable base such as for example N-methylmorpholine, TEA or DIPEA may be necessary. In certain cases, the activated acid derivative might be isolated prior to reaction with the appropriate amine. Amide formation may also be accomplished via the acid halide (which can be formed from a carboxylic acid by reaction with e.g. oxalyl chloride, thionyl chloride or sulfuryl chloride), mixed acid anhydride (which can be formed from a carboxylic acid by reaction with e.g. isobutylchloroformate), imidazolide (which can be formed from a carboxylic acid by reaction with e.g. carbonyldiimidazole) or azide (which can be formed from a carboxylic acid by reaction with e.g. diphenylphosphorylazide).

Carboxylic acids of general formula 8 in turn may be obtained from carboxylic esters of formula 7 by saponification with inorganic bases such as lithium hydroxide, potassium hydroxide or sodium hydroxide in a suitable solvent such as methanol, THF, water or mixtures thereof at temperatures between 0° C. and the boiling point of the solvent (mixture), typically at room temperature. Alternatively, carboxylic acids of general formula 8 may be directly formed from aryl bromides of general formula 5 under palladium catalyzed carbonylation conditions. Thus, bromides of formula 5 may be reacted in a suitable solvent such as for example dimethyl sulfoxide in the presence of a carbon monoxide source such as for example molybdenum hexacarbonyl or under a carbon monoxide atmosphere at pressures between 1 and 20 bar and in the presence of a palladium catalyst system such as for example palladium(II) acetate/1,1′-bis(diphenylphosphino)ferrocene and a base such as potassium acetate at temperatures between room temperature and the boiling point of the solvent, preferably at 100° C.

Carboxylic esters of general formula 7 may be synthesized from aryl bromides of formula 5 by reaction with an appropriate alcohol under palladium catalyzed carbonylation conditions. Bromides of formula 5 might be reacted in a polar aprotic solvent such as for example dimethylsulfoxide with an appropriate alcohol such as methanol in the presence of a carbon monoxide source such as for example molybdenum hexacarbonyl or under a carbon monoxide atmosphere at pressures between 1 and 20 bar and in the presence of a suitable palladium catalyst such as bis(triphenylphosphine) palladium(II) dichloride and a base such as for example triethylamine at temperatures between room temperature and the boiling point of the solvent, preferably at 100° C.

Alternatively, amides of general formula 6 may be directly synthesized from aryl bromides of formula 5 by reaction with appropriate amines HN(R^{5a})(R^{5b}) (9) under palladium catalyzed carbonylation conditions. For this carbonylation all processes that are known to the person skilled in the art may be applied. Bromides of formula 5 can be reacted in a polar aprotic solvent such as for example dioxane with an appropriate amine in the presence of a carbon monoxide source such as for example molybdenum hexacarbonyl or under a carbon monoxide atmosphere at pressures between 1 and 20 bar and in the presence of a palladium catalyst such as for example palladium(II) acetate and a base such as sodium carbonate at temperatures between room temperature and the boiling point of the solvent, preferably at 110° C. It might be necessary to add a ligand such as tri-tert-butylphosphonium tetrafluoroborate to the mixture.

Aryl bromides of general formula 5 in turn may be formed from indolines of general formula 4 by reaction with electrophiles of formula R₂—SO₂—Cl in an organic solvent such as dichloromethane, 1,2-dichloroethane or acetonitrile in the presence of a tertiary amine base such as triethylamine or DIPEA and optionally in the presence of 4-dimethylaminopyridine at temperatures between room temperature and the boiling point of the solvent, typically at 80° C. Alternatively, indolines of general formula 4 may be reacted with electrophiles of formula R₂—SO₂—Cl without additional solvent in the presence of a tertiary base such as triethylamine or pyridine at room temperature to give aryl bromides of general formula 5. In the above procedures, electrophiles R₂—SO₂—Cl are either commercially available, known compounds or may be formed from known compounds by known methods by a person skilled in the art.

Indolines of general formula 4 may be synthesized from suitably functionalized indolenines of general formulae 3a or 3b by either reduction (3a to 4) or addition of a nucleophile (3b to 4). For reduction, the indolenines 3a may be reacted in a suitable organic solvent such as for example methanol in the presence of a reducing agent such as for example sodium borohydride, sodium (triacetoxy)borohydride or sodium cyanoborohydride at temperatures between 0° C. and the boiling point of the solvent, typically at room temperature. In case of a nucleophilic addition, the indolenines 3b may be reacted in a suitable organic solvent such as for example THF with a nucleophile R₁-M (where M is a metallic species; R₁-M is for example a Grignard reagent) at temperatures between 0° C. and the boiling point of the solvent, typically at room temperature (see WO06/090261, pp. 67-68 for a similar procedure). It might be necessary to add a Lewis acid such as boron trifluoride diethyl etherate to the mixture.

Alternatively, 3b may be reacted in a suitable organic solvent such as for example toluene with a Grignard reagent R₁-M in the presence of copper(I) chloride at temperatures between room temperature and the boiling point of the solvent, typically at 120° C. to give indolines of general formula 4 (see J. Chem. Soc. Perkin Trans. 1, 1988, 3243-3247).

Indolenines of general formulae 3a or 3b may be obtained from suitably functionalized carbonyl compounds of general formulae 2a or 2b and a phenylhydrazine of formula 1 by condensation to give a hydrazone intermediate and a subsequent cyclization reaction (Fischer indole synthesis) in an organic solvent such as for example chloroform or acetic acid and in the presence of a suitable acid such as for example trifluoroacetic acid or hydrochloric acid at temperatures between 0° C. and the boiling point of the solvent (see for example Liu et al., Tetrahedron 2010, 66, 3, 573-577 or WO10/151737, p. 224 for similar procedures). In the above procedures, carbonyl compounds of general formulae 2a or 2b and phenyl-hydrazines of general formula 1 are either commercially available, known compounds or may be formed from known compounds by known methods by a person skilled in the art.

The obtained indolines of general formula 6 may be chiral and may be separated into their diastereomers and/or enantiomers by chiral HPLC.

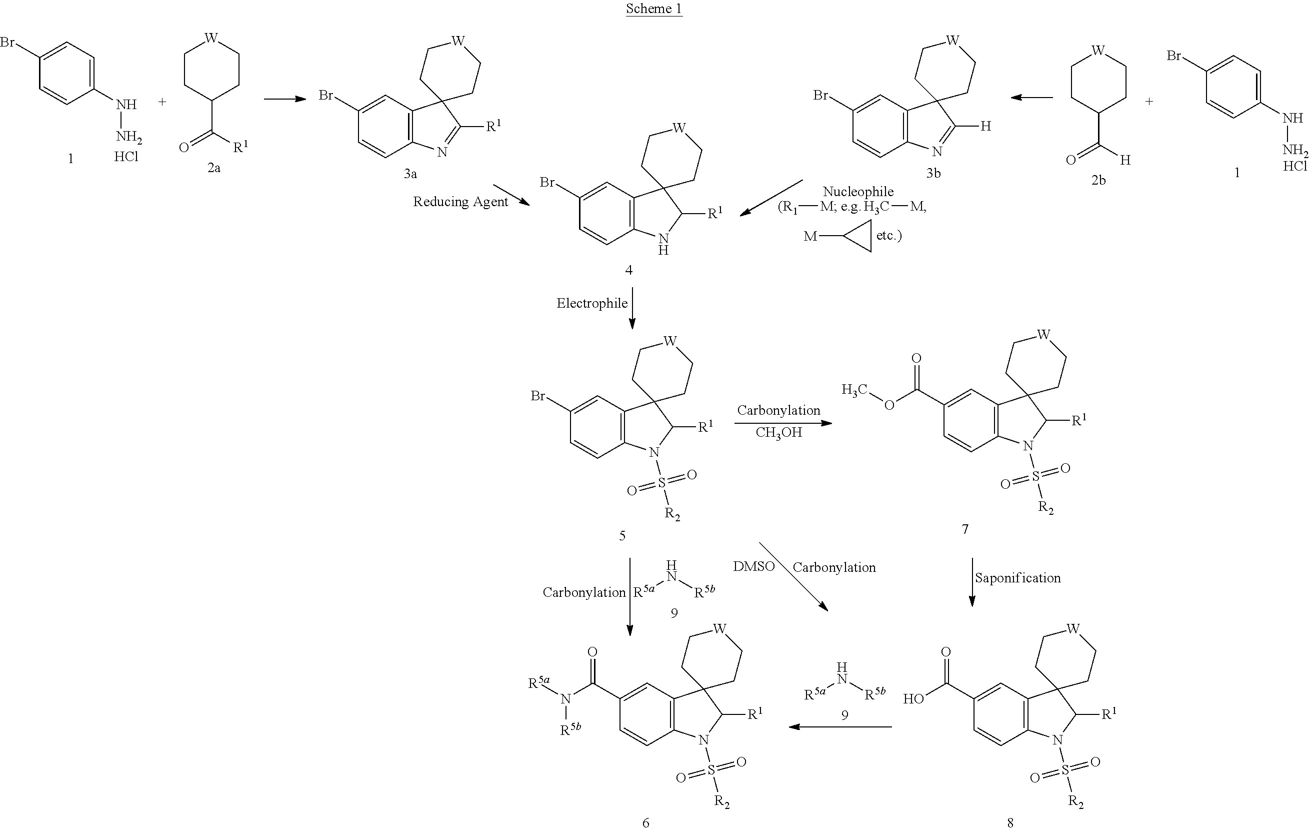

Scheme 1

General procedures for the preparation of compounds of general formula 6; W, R¹, R², R^{5a}, and R^{5b} are as defined in the descrpton and claims of ths invention. The procedures are favorable for the synthesis of compounds of general formula (l) wherein R³ is C(O)N(R^{5a})(R^{5b}).

Instead of using carbonyl compounds of general formula 2b in the indolenine synthesis (see Scheme 1) enol ethers of general formula 10 can be applied in certain cases to obtain indolenines of general formula 3b as depicted in Scheme 2. The reaction conditions are comparable to those described in Scheme 1 for the syntheses of 3b from 1 and 2b. Enol ethers of formula 10 are either commercially available, known compounds or may be formed from known compounds by known methods by a person skilled in the art.

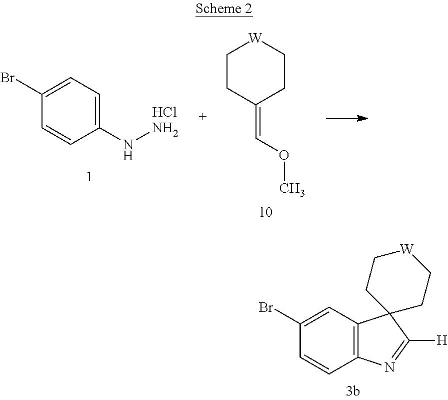

Scheme 2

General procedure for the preparation of compounds of general formula 3b; W is as defined in the description and claims of this invention.

In case of spirotetrahydrothiopyranes the sulfur atom might be oxidized as depicted in Scheme 3. Sulfones of general formula 13 may be obtained from suitably functionalized spirotetrahydrothiopyranes of general formula 11 by twofold oxidation applying peroxides. Thus, spirotetrahydrothiopyranes of formula 11 may be reacted in organic solvents such as for example dichloromethane or acetonitril with peroxides such as for example 3-chloroperoxybenzoic acid or urea hydrogen peroxide in the presence of trifluoroacetic anhydride at temperatures between 0° C. and the boiling point of the solvent, preferably at room temperature. It might be necessary to add trifluoroacetic anhydride to the mixture. Alternatively, sulfones of formula 13 may be synthesized from sulfoxides of general formula 12 under similar reaction conditions as described for the syntheses of 13 from 11.

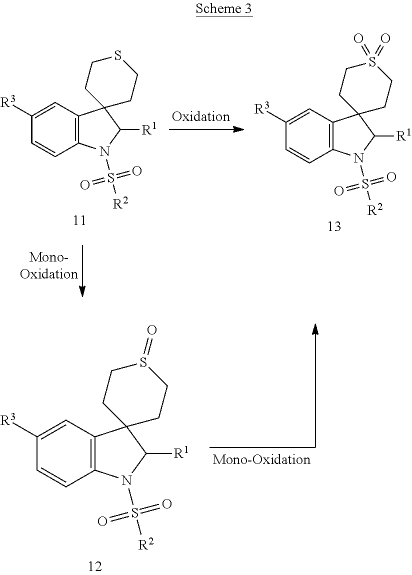

Scheme 3

General procedures for the preparation of compounds of general formula 12 and 13; R¹, R², and R³ are as defined in the description and claims of this invention. The procedures are favorable for the synthesis of compounds of general formula (I) wherein W is SO or SO₂.

Sulfoxides of general formula 12 may be obtained from spirotetrahydrothiopyranes of general formula 11 by mono-oxidation in an organic solvent such as for example acetonitrile with periodic acid and a catalytic amount of iron(III) chloride at temperatures between 0° C. and the boiling point of the solvent, preferably at room temperature.

Compounds of general formula 20 may be synthesized according to the procedures shown in Scheme 4. The compounds of formulae 20, 21 and 22 can be obtained in an analogous way as described for the compounds of formulae 6, 7 and 8 in Scheme 1.

Sulfones of general formula 19 may be synthesized from compounds of general formula 18 by oxidation with peroxides. The procedures are analogous to those described for the syntheses of 13 from 11 in Scheme 3.

Sulfonamides of general formula 18 may be obtained from suitably functionalized indolines of general formula 17 by reaction with electrophiles of formula R₂—SO₂—Cl as described for the syntheses of 5 from 4 in Scheme 1.

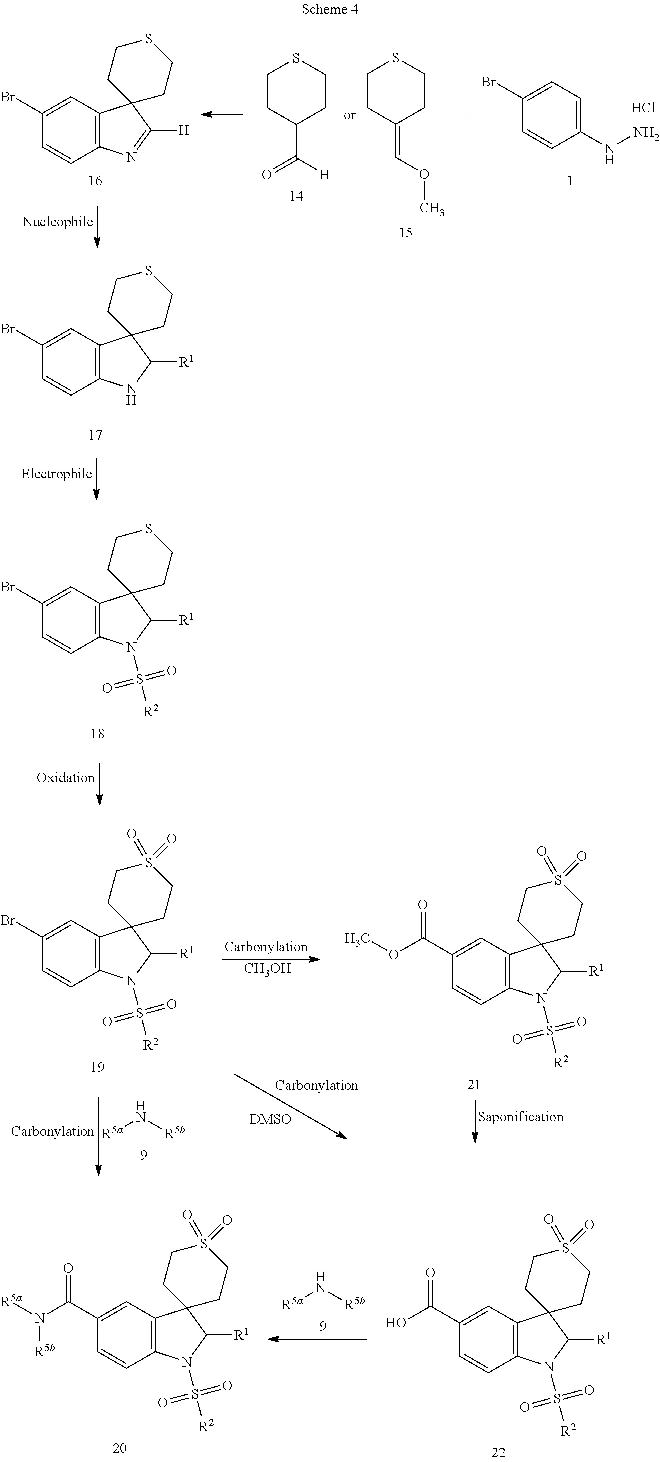

Scheme 4

General procedures for the preparaton of compounds of general formula 20; R¹, R², R^{5a}, and R^{5b} are as defined in the description and claims of this invention. The procedures are favorable for the synthesis of compounds of general formula (I) wheren W is SO₂, R¹ is ≠ H and R³ is C(O)N(R^{5a})(R^{5b}).

Indolines of general formula 17 may be synthesized from suitably functionalized indolenines of general formula 16 by reaction in a suitable organic solvent such as for example THF with a nucleophile R₁-M (where M is a metallic species; R₁-M is for example a Grignard reagent) in the presence of a Lewis acid such as boron trifluoride diethyl etherate at temperatures between 0° C. and the boiling point of the solvent, typically at room temperature. Alternatively, 16 may be reacted in a suitable organic solvent such as for example toluene with a Grignard reagent R-M in the presence of copper(I) chloride at temperatures between room temperature and the boiling point of the solvent, typically at 120° C. (see J. Chem. Soc. Perkin Trans. 1, 1988, 3243-3247).

Indolenines of general formula 16 may be obtained from suitably functionalized carbonyl compounds of general formula 14 and a phenylhydrazine of formula 1 by condensation in an analogous way as described for the syntheses of 3b from 1 and 2b in Scheme 1. Alternatively, indolenines of general formula 16 may be synthesized from suitably functionalized enol ethers of general formula 15 and a phenylhydrazine of formula 1 as described in Scheme 2.

It is obvious to the person skilled in the art that the oxidations as exemplified in Scheme 3 and Scheme 4 can be done at different stages of the syntheses to obtain compounds of the present invention.

Compounds of general formula (I) (e.g. amides, ureas, carbamates) may be synthesized according to the procedures depicted in Scheme 5 from suitably functionalized anilines of general formula 25 by reaction with electrophiles. Thus, anilines of formula 25 may be reacted with appropriate carboxylic acids to form amides (I). For amide formation, however, all processes that are known from peptide chemistry to the person skilled in the art may be applied (see description for the synthesis of compounds of formula 6 from 8 and 9 in Scheme 1).

Furthermore, anilines of general formula 25 can be reacted with appropriate isocyanates in a suitable organic solvent such as for example DMF and optionally in the presence of a tertiary amine base such as triethylamine or DIPEA at temperatures between 0° C. and the boiling point of the solvent to form ureas (I).

Additionally, anilines of general formula 25 can be reacted with appropriate chloroformates or 4-nitrophenylcarbonates in a suitable organic solvent such as for example THF and in the presence of a tertiary amine base such as triethylamine or DIPEA at temperatures between 0° C. and the boiling point of the solvent to form carbamates (I).

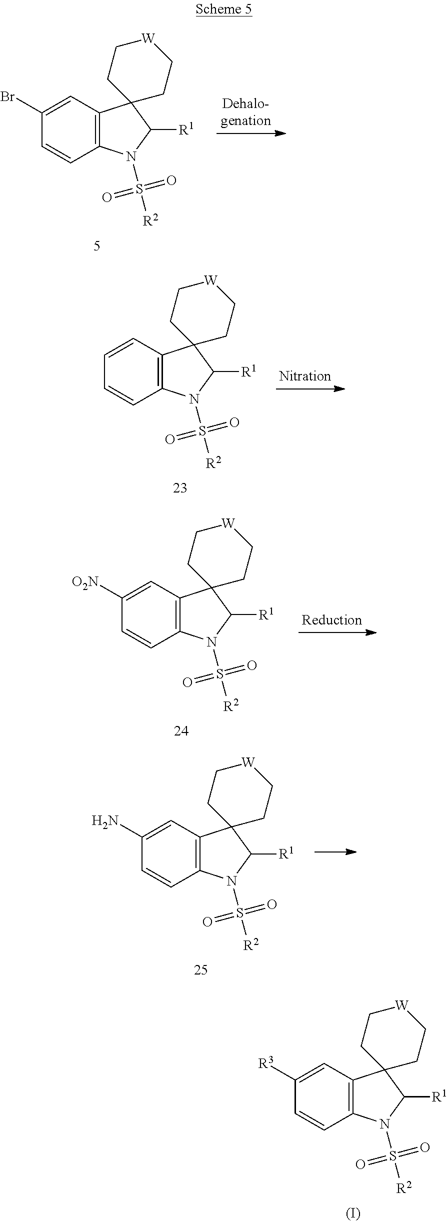

Scheme 5

General procedures for the preparation of compounds of general formula (I); W, R¹, R² and R³ are as defined in the description and claims of this invention. The procedures are favorable for the synthesis of compounds of general formula (I) wheren R³ is N(H)C(O)R⁶ or N(H)C(O)N(R^{5a})(R^{5b}) or N(H)C(O)OR⁷.

Anilines of general formula 25 can be obtained from nitroarenes of general formula 24 by reduction. For reduction, all processes that are known to the person skilled in the art may be applied. Nitroarenes 24 may be hydrogenated under an atmosphere of hydrogen at pressures between 1 bar and 100 bar in a suitable solvent such as for example ethyl acetate, methanol or ethanol or by leading hydrogen through the solution and in the presence of a metal catalyst such as for example palladium on charcoal at temperatures between 0° C. and the boiling point of the solvent, typically at room temperature. The addition of a suitable acid such as for example hydrochloric acid or acetic acid may be necessary.

Nitroarenes of general formula 24 can be synthesized from compounds of general formula 23 by regioselective nitration. For nitration, all processes that are known to the person skilled in the art may be applied. Compounds of formula 23 may be reacted with a mixture of concentrated nitric acid and sulfuric acid or with a mixture of concentrated nitric acid and acetic acid at temperatures between 0° C. and the boiling point of the solvent, typically at room temperature.

Compounds of general formula 23 may be obtained from aryl bromides of general formula 5 by dehalogenation. For dehalogenation, the bromides of formula 5 may be hydrogenated under an atmosphere of hydrogen at pressures between 1 bar and 100 bar in a suitable solvent such as for example ethyl acetate, tetrahydrofurane, methanol, ethanol or mixtures thereof or by leading hydrogen through the reaction mixture and in the presence of a metal catalyst such as for example palladium on charcoal at temperatures between 0° C. and the boiling point of the solvent, typically at room temperature.

Aryl bromides of general formula 5 are obtainable according to the procedures described in Scheme 1.

Alternatively, anilines of general formula 25 can be obtained from carboxylic acids of general formula 8 by a two step protocol involving Curtius rearrangement followed by deprotection as shown in Scheme 6. For deprotection of tert-butyloxycarbonyl (Boc) groups, all processes that are known to the person skilled in the art may be applied. The protected aniline of general formula 26 may be reacted in an organic solvent such as for example dichloromethane, diethyl ether or 1,4-dioxane with an acid such as trifluoroacetic acid or hydrochloric acid at temperatures between 0° C. and the boiling point of the solvent, preferably at room temperature to give 25.

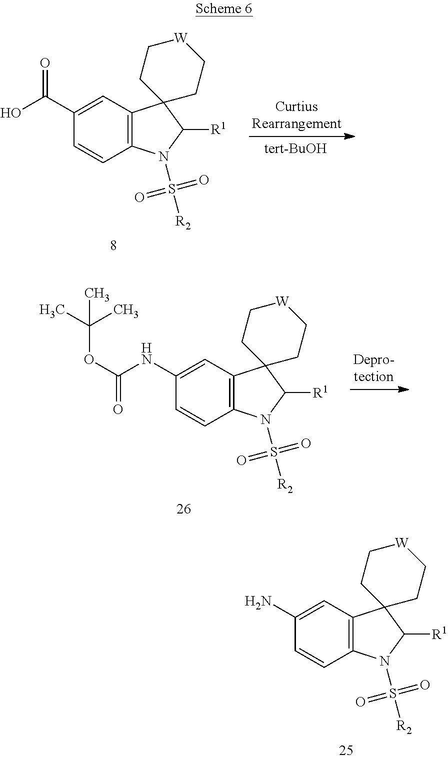

Scheme 6

Alternative procedures for the preparation of compounds of general formula 25 starting from carboxylic acids of general formula 8; W, R¹ and R², are as defined in the description and claims of this invention.

The protected aniline of general formula 26 can be obtained from carboxylic acids of general formula 8 by reaction in an organic solvent such as tert-butanol with an azide source such as for example diphenylphosphoryl azide in the presence of an organic base such as for example triethylamine at temperatures between 40° C. and 150° C., preferably at 85° C. It might be necessary to add molecular sieves to the mixture.

### General Procedures

In the subsequent paragraphs detailed general procedures for the synthesis of key intermediates and compounds of the present invention are described.

General Procedure 1 (GP 1): Indolenine formation (3a and 3b, Schemes 1 and 2)

Method 1 (GP 1.1): Similar to Liu et al., Tetrahedron 2010, 66, 3, 573-577 or WO10/151737, p. 224.

To a stirred solution of 1 eq. of hydrazine 1 and 1 eq. of carbonyl compound 2a or 2b or enol ether 10 in chloroform at 0° C., 3.3 eq. of trifluoroacetic a cid are added dropwise. The reaction mixture is heated to 50° C. until TLC and/or LCMS indicate complete consumption of the starting material (18 h) and then cooled to room temperature. An aqueous solution of ammonia (25%) is carefully added to reach a pH of ˜8. The mixture is poured into water and extracted with dichloromethane. The combined organic layers are washed with water, dried with sodium sulfate and the solvents removed in vacuo. The crude product is taken to the next step without further purification.

Method 2 (GP 1.2): Indolenine formation in acetic acid/aq. hydrochloric acid To a stirred solution of 1 eq. of hydrazine 1 in acetic acid (2 mL/mmol) 1 eq. of concentrated hydrochloric acid (aq.) is added at rt. After 5 minutes of stirring, 1 eq. of carbonyl compound 2a or 2b or enol ether 10 is added at rt, the reaction mixture heated to 100° C. until TLC and/or LCMS indicate (nearly) complete consumption of the starting material (4-24 h) and then cooled to room temperature. An aqueous solution of ammonia (25%) is carefully added to reach a pH of ˜8. The mixture is poured into water and extracted with dichloromethane. The combined organic layers are washed with water, dried with sodium sulfate and the solvents removed in vacuo. The crude product is taken to the next step without further purification.

General Procedure 2 (GP 2): Reduction of indolenine (3a→4, Scheme 1) To a stirred solution of indolenine 3a in methanol, 4 eq. of sodium borohydride are carefully added at rt. The reaction is stirred at rt until TLC and/or LCMS indicate complete consumption of the starting material (1 h) and then concentrated in vacuo. The residue is taken up with water, acidified with aq. hydrochloric acid (1 M) to a pH of ˜5 and extracted with ethyl acetate. The combined organic layers are washed with brine, dried with sodium sulfate and the solvents removed in vacuo. The crude product is purified by flash chromatography or preparative HPLC.

General Procedure 3 (GP 3): Grignard reaction (Nucleophile addition, 3b→4, Scheme 1) Similar to WO06/090261, pp. 67-68.

To a stirred solution of indolenine 3b in THF, 1 eq. of boron trifluoride diethylether complex is added dropwise at 0° C. After 5 min of stirring, 3 e q. of the corresponding Grignard reagent (commercial solution in THF or prepared from the respective alkyl bromide according to standard procedures) are added dropwise, keeping the temperature of the mixture at 5-10° C. The mixture is allowed to warm to room temperature and stirred until TLC and/or LCMS indicate complete consumption of the starting material (3 h). Then sat. aqueous ammonium chloride solution is added and the mixture partitioned between ethyl acetate and water. The aqueous phase is extracted with ethyl acetate, the combined organic phases are washed with brine, dried with sodium sulfate, concentrated and purified via flash chromatography (SiO₂-hexane/ethylacetate).

General Procedure 4 (GP 4): Sulfonamide formation (4→5, Scheme 1)

Method 1 (GP 4.1): Sulfonamide formation in 1,2-dichloroethane

To a solution of indoline 4 in 1,2-dichloroethane 2 eq. of sulfonyl chloride and 5 eq. of triethylamine are added at rt and the mixture is stirred at 80° C. for 18-24 h. If needed, further 2 eq. of sulfonyl chloride and 3 eq of triethylamine may be added and the mixture is stirred for additional 18 h. The reaction mixture is partitioned between water and dichloromethane, extracted with dichloromethane, the combined organic layers are washed with water, dried with sodium sulfate, concentrated and purified via flash chromatography (SiO₂-hexane/ethylacetate).

Method 2 (GP 4.2): Sulfonamide formation in pyridine

A mixture of indoline 4, 2 eq. of sulfonyl chloride and 6 eq. of pyridine is stirred at rt for 18-24 h. The reaction mixture is partitioned between water and dichloromethane, extracted with dichloromethane, the combined organic layers are washed with water, dried with sodium sulfate, concentrated and purified via flash chromatography (SiO₂-hexane/ethyl acetate).

General Procedure 5 (GP 5): Oxidation to sulfone (11→13, Scheme 3)

Method 1 (GP 5.1): Oxidation with mCPBA

To a solution of sulfide 11 in dichloromethane, 3 eq. of 3-chloroperoxybenzoic acid are added at 0° C. The mixture is stirred until TLC and/or LCMS indicate complete consumption of the starting material (4 h) and then partitioned between dichloromethane and sat. aqueous sodium hydrocarbonate solution. The organic layer is washed with sodium hydrocarbonate solution, dried with sodium sulfate and concentrated in vacuo. The crude product is purified via flash chromatography (SiO₂-hexane/ethyl acetate).

Method 2 (GP 5.2): Oxidation with urea hydrogen peroxide

6 Eq. trifluoroacetic anhydride are dissolved in acetonitril (5-6 mL/mmol) at 0° C. and 8 eq. of urea hydrogen peroxide are slowly added. After 20 min stirring at rt, a solution of 1 eq. of sulfide 11 in acetonitrile (3.5 mL/mmol) is added dropwise and the mixture stirred for ca. 2 h at rt. In case of incomplete conversion, further up to 8 eq. of urea hydrogen peroxide and the according amount of trifluoroacetic anhydride may be added. After complete conversion, the mixture is partitioned between water and dichloromethane. The aqueous layer is extracted with dichloromethane, the combined organic layers are washed with water and dried with sodium sulfate. The solvents are removed in vacuo and the crude product is purified by flash chromatography to obtain the desired sulfone.

Method 3 (GP 5.3): Oxidation with Oxone®

To a solution of sulfide 11 in a mixture of tetrahydrofurane and methanol (1:1), a solution of 4 eq of Oxone® in water (0.15-0.35 M) is added at 0° C. The mixture is stirred at 0° C. until TLC and/or LCMS indicate complete consumption of the starting material (2 h) and then partitioned between water and ethyl acetate. The layers are separated, the aqueous layer is extracted with ethyl acetate, the combined organic layers washed with brine, dried with sodium sulfate and the solvents removed in vacuo. The obtained crude product is purified via flash chromatography (SiO₂-hexane/ethyl acetate).

General Procedure 6 (GP 6): Carbonylation to yield methylester (5→7, Scheme 1)

The aryl bromide 5 is placed into a steel autoclave under argon atmosphere and dissolved in a 10:1 mixture of methanol and dimethyl sulfoxide (ca. 30 mL/mmol). 0.2 eq. of trans-bis(triphenylphosphine) palladium(II) dichloride and 2.5 eq. of triethylamine are added and the mixture is purged 3 times with carbon monoxide. The mixture is stirred for 30 min at 20° C. under a carbon monoxide pressure of ca. 9.5 bar. The autoclave is set under vacuum again, then a carbon monoxide pressure of ca. 8.6 bar is applied and the mixture heated to 100° C. until TLC and/or LCMS indicate complete consumption of the starting material (22 h), yielding a maximum pressure of ca. 12.2 bar. The reaction is cooled to rt, the pressure released and the reaction mixture concentrated in vacuo and redissolved in ethyl acetate/water. The layers are separated, the aqueous phase extracted with ethyl acetate, the combined organic layers washed with water and brine, then dried with sodium sulfate and the solvents removed in vacuo. The crude product is purified by flash chromatography (SiO₂-hexane/ethyl acetate).

General Procedure 7 (GP 7): Saponification of ester (7→8, Scheme 1)

The methyl ester 7 is dissolved in a 1:1 mixture of THF and a 2M aqueous lithium hydroxide solution (ca. 30 mL/mmol) and stirred at rt until TLC and/or LCMS indicate complete consumption of the starting material (18 h). The mixture is set to pH 4 by addition of 2M aqueous hydrochloric acid and extracted with ethyl acetate. The combined organic layers are washed with brine, dried with sodium sulfate and concentrated in vacuo. The product is used without further purification.

General Procedure 8 (GP 8): Carbonylation to yield carboxylic acid (5→8, Scheme 1)

The aryl bromide 5 is placed into a steel autoclave under argon atmosphere and dissolved in dimethyl sulfoxide (ca. 25 mL/mmol). 5 mol % of palladium(II) acetate, 0.2 eq. of 1,1′-bis(diphenylphosphino)ferrocene and 4 eq. of potassium acetate are added and the mixture is purged 3 times with carbon monoxide. The mixture is stirred for 30 min at 20° C. under a carbon monoxide pressure of ca. 10.5 bar. The autoclave is set under vacuum again, then a carbon monoxide pressure of ca. 11 bar is applied and the mixture heated to 100° C. until TLC and/or LCMS indicate complete consumption of the starting material (22 h), yielding a maximum pressure of ca. 13.5 bar. The reaction is cooled to rt, the pressure released and the reaction mixture given to a mixture of 2 M HCl_{aq} in ice-water. After stirring for 20 min, the formed precipitate is filtered off, washed with water and redissolved in dichloromethane. The organic layer is washed with water, dried with magnesium sulfate and the solvent removed in vacuo. The obtained crude product is taken to the next step without further purification.

General Procedure 9 (GP 9): Amide formation (8→6, Scheme 1)

Method 1 (GP 9.1): Amide formation in situ

The carboxylic acid 8 is dissolved in DMF and 2 eq. of the corresponding amine component, 1.5 eq. of HATU and 3 eq. of triethylamine are added. The reaction mixture is stirred at rt until TLC and/or LCMS indicate complete consumption of the starting material (2 h), then water is added. The formed precipitate is filtered off, washed with water and dried in a vacuum drying cabinet at 40° C. If appropriate, the product is purified by preparative HPLC.

Method 2 (GP 9.2): Amide formation after isolation of active ester (HOAt ester)

The carboxylic acid 8 is dissolved in DMF, 1.5 eq. of HATU and 1.5 eq. of triethylamine are added. The reaction mixture is stirred at rt until TLC and/or LCMS indicate complete consumption of the starting material (2-3 h), then water is added. The formed precipitate is filtered off, washed with water, dissolved in dichloromethane, dried and concentrated in vacuo to give the HOAt ester.

The HOAt ester and 1.5 eq. of the corresponding amine component are stirred in acetonitrile or a mixture of acetonitrile and N-methyl-2-pyrrolidone at 55-80° C. until TLC and/or LCMS indicate complete consumption of the HOAt ester (1-30 h). Then the reaction mixture is partitioned between ethyl acetate and water. The layers are separated, the water phase extracted with ethyl acetate, the combined organic layers washed with water and brine, then dried with sodium sulfate and the solvents removed in vacuo. If appropriate, the product is purified by preparative HPLC or flash chromatography.

General Procedure 10 (GP 10): Carbonylation to yield amides directly (5→6, Scheme 1)

To a solution of aryl bromide 5 in 1,4-dioxane (containing ca. 1% water) 3 eq. of the corresponding amine, 1 eq. of molybdenum hexacarbonyl, 3 eq. of sodium carbonate, 0.1 eq. of tri-tert-butylphosphonium tetrafluoroborate and 0.1 eq. of palladium(II) acetate are added. The reaction mixture is vigorously stirred at 120-140° C. until TLC and/or LCMS indicate complete consumption of the starting material (18 h). Alternatively, microwave irradiation (200 W, 20 min, 140° C., 1.2 bar) can be applied. The mixture is cooled to rt, solids are filtered off and rinsed with ethyl acetate. The filtrate is washed with water and brine, dried with sodium sulfate and concentrated in vacuo. The crude product is purified by flash chromatography (SiO₂-hexane/ethyl acetate) and if appropriate additionally by preparative HPLC.

General Procedure 11 (GP 11): Oxidation sulfide→sulfoxide (11→12, Scheme 3)

To a solution of sulfide 11 in acetonitrile 0.13 eq. of iron(III) chloride are added at rt. After min stirring, 1.1 eq. of periodic acid is added and the mixture stirred for further 45 min. The mixture is partitioned between water and ethyl acetate. The pH is adjusted to ˜pH 10 by the addition of aqueous sat. sodium hydrocarbonate solution. The layers are separated, the aqueous phase extracted with ethyl acetate, the combined organic layers are washed with brine, dried with sodium sulfate and the solvents evaporated. The crude product is purified by flash chromatography or preparative HPLC.

General Procedure 12 (GP 12): Dehalogenation (5→23, Scheme 5)

To the aryl bromide 5 in ethanol (ca. 10 mL/mmol) or a mixture of ethanol and tetrahydrofurane (3:1) 0.3 eq. of palladium on charcoal (10% Pd/C; contains 50% of water) are added at rt and hydrogen gas is led into the mixture until TLC and/or LCMS indicate complete consumption of the starting material (2-3 h). The catalyst is filtered off and rinsed with ethanol and THF. The filtrate is concentrated in vacuo and the residue partitioned between dichloromethane and water. The layers are separated, the aqueous phase extracted with dichloromethane, the combined organic layers are washed with sat. sodium hydrocarbonate solution and brine, then dried with magnesium sulfate and the solvents removed in vacuo. The obtained crude product is taken to the next step without further purification.

General Procedure 13 (GP 13): Nitration (23→24, Scheme 5)

To a solution of indoline 23 in acetic acid (ca. 6.5 mL/mmol) 30 eq. of concentrated nitric acid are carefully added at rt. The reaction mixture is stirred at rt until TLC and/or LCMS indicate complete consumption of the starting material (2-3 h) and then dropwise added to a sat. sodium hydrocarbonate solution (ca. 140 mL/mmol). After the gas evolution has ceased the aqueous phase is extracted with ethyl acetate, the combined organic layers are washed with sat. sodium hydrocarbonate solution and brine, dried with magnesium sulfate and the solvents removed in vacuo. The obtained crude product is taken to the next step without further purification.

General Procedure 14 (GP 14): Reduction NO₂→NH₂ (24→25, Scheme 5)

To the nitroarene 24 in ethyl acetate (ca. 20 mL/mmol) 0.1 eq of palladium on charcoal (10% Pd/C) is added at rt and hydrogen gas is led into the mixture until TLC and/or LCMS indicate complete consumption of the starting material (2-5 h). The catalyst is filtered off and rinsed with ethyl acetate. The filtrate is concentrated in vacuo and the obtained crude product purified by flash chromatography (SiO₂-hexane/ethyl acetate).

General Procedure 15 (GP 15): Reaction of anilines with electrophiles (25→(I), Scheme 5)

Method 1 (GP 15.1): Amide formation

The respective carboxylic acid (1.5 eq.) is dissolved in DMF and 1 eq. of aniline 25, 1.5 eq. of HATU and 1.5 eq. of triethylamine are added. The reaction mixture is stirred at rt until TLC and/or LCMS indicate complete consumption of the starting material (8-24 h), then water is added. The formed precipitate is filtered off, washed with water and taken up with dichloromethane. The organic phase is washed with water, dried with magnesium sulfate and concentrated in vacuo. If appropriate, the product is purified by flash chromatography (SiO₂-hexane/ethyl acetate) or preparative HPLC.

### Synthesis of Key Intermediates

### Intermediate A.1

### Preparation of 5-bromo-2′,3′,5′,6′-tetrahydrospiro[indole-3,4′-thiopyran]

### Access Via Carbonyl Compound: Step 1a Swern Oxidation

### Preparation of 3,4,5,6-tetrahydro-2H-thiopyran-4-carbaldehyde

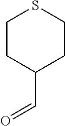

1.4 eq. oxalyl chloride (6.72 g, 52.9 mmol) were dissolved in 200 mL methylene chloride and the solution cooled to −65° C. 2 eq. dimethyl sulfoxide (5.91 g, 75.6 mmol), dissolved in 30 mL methylene chloride were added dropwise within 10 min, so that the temperature didn't exceed −50° C. After 15 min, 1 eq. tetrahydrothiopyran-4-methanol (5.00 g, 37.8 mmol), dissolved in 30 mL methylene chloride, were added dropwise within 5 min at max. −45° C. The mixture was stirred for 1 h, warming to −30° C. 3 eq. triethylamine (11.5 g, 113 mmol) were added dropwise and the mixture was allowed to warm up to room temperature. After stirring 1 h, the mixture was poured into water and extracted with methylene chloride. The combined organic layers were washed with water, dried with sodium sulfate, the solvents removed in vacuo and the crude product (5.70 g, 98%) was directly put forward to the next step.

### Access Via Enol Ether: Step 1b Wittig Reaction (WO09/007747, Pp. 60-61)

### Preparation of 4-(methoxymethylene)-3,4,5,6-tetrahydro-2H-thiopyran

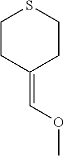

A mixture of (methoxymethyl)triphenylphosphonium chloride (885 g, 2.58 mol, 1.50 eq.) in THF (1300 mL) was cooled to −50° C. and LDA (1.29 L of a 2 M solution in THF/Heptane/Ethylbenzene, 2.58 mol, 1.50 eq.) was added dropwise keeping the temperature below −20° C. After 15 min at −20° C. the deep red reaction mixture was cooled to −40° C. and a solution of tetrahydrothiopyran-4-one (200 g, 1.72 mol, 1.00 eq) in THF (1000 mL) was added dropwise. After 15 min at −40° C. the mixture was allowed to reach rt and was stirred overnight. The reaction mixture was filtered, concentrated in vacuo and filtered again. The obtained filtrate was purified by distillation (B.p. 60° C., 0.02 mbar) to give the title compound (125 g, 50%). ¹H-NMR (300 MHz, CDCl₃): Shift [ppm]=2.27-2.30 (m, 2H), 2.52-2.55 (m, 2H), 2.59-2.62 (m, 4H), 3.55 (s, 3H), 5.82 (s, 1H). UPLC-MS (ESI+): [M+H]⁺=145.

### Step 2 Fischer Indole Synthesis

### Preparation of 5-bromo-2′,3′,5′,6′-tetrahydrospiro[indole-3,4′-thiopyran]

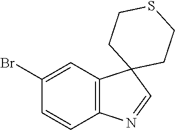 [SMILES: Brc1ccc2c(c1)C1(C=N2)CCSCC1]

According to GP 1.1 1 eq. of 4-bromo-phenylhydrazine hydrochloride (8.96 g, 40.1 mmol) and 1 eq. 3,4,5,6-tetrahydro-2H-thiopyran-4-carbaldehyde (5.80 g, 40 mmol) or, alternatively, 1 eq. of 4-(methoxymethylene)-3,4,5,6-tetrahydro-2H-thiopyran were dissolved in 250 mL chloroform. The solution was cooled to 0° C. and 3.3 eq. trifluoroacetic acid (15.8 g) were added dropwise. The reaction was heated to 50° C. for 18 h, then cooled to room temperature. An aqueous solution of ammonia (25%) was carefully added to reach a pH of about 8. The mixture was poured into water and extracted with methylene chloride. The combined organic layers were washed with water, dried with sodium sulfate and the solvents removed. The product was put to the next step without further purification. UPLC-MS (ESI+): [M+H]⁺=282/284 (Br isotope pattern).

### Intermediate A.2

### Preparation of 5-bromo-2′,3′,5′,6′-tetrahydrospiro[indole-3,4′-pyran]

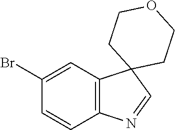 [SMILES: Brc1ccc2c(c1)C1(C=N2)CCOCC1]

Intermediate A.2 was prepared in analogy to intermediate A.1 according to GP 1.1 starting from 3,4,5,6-tetrahydro-2H-pyran-4-carbaldehyde (CAS No. [50675-18-8]) and 4-bromo-phenylhydrazine hydrochloride. UPLC-MS (ESI+): [M+H]⁺=266/268 (Br isotope pattern).

### Intermediate A.3

### Preparation of 5-bromo-2-cyclopropyl-2′,3′,5′,6′-tetrahydrospiro[indole-3,4′-pyran]

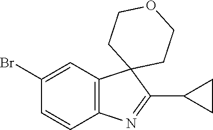 [SMILES: Brc1ccc2c(c1)C1(CCOCC1)C(C1CC1)=N2]

Intermediate A.3 was prepared according to GP 1.2 starting from cyclopropyl-(tetrahydro-2H-pyran-4-yl)-methanone (CAS No. [1340079-14-2]) and 4-bromo-phenylhydrazine hydrochloride. UPLC-MS (ESI+): [M+H]⁺=306/308 (Br isotope pattern).

### Intermediate B.1

### Preparation of 5-bromo-2-cyclopropyl-1,2,2′,3′,5′,6′-hexahydrospiro[indole-3,4′-thiopyran]

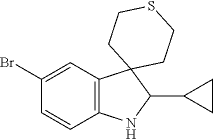 [SMILES: Brc1ccc2c(c1)C1(CCSCC1)C(C1CC1)N2]

According to GP 3 intermediate A.1 (8.82 g, 27.2 mmol), 81.6 mmol cyclopropylmagnesium bromide (0.5 M in THF) and 1 eq (3.86 g) borontrifluoride etherate were reacted in 100 mL THF to yield 3.50 g (32%) of intermediate B.1. ¹H-NMR (300 MHz, DMSO-d6): Shift [ppm]=0.08-0.19 (m, 1H), 0.32-0.42 (m, 2H), 0.43-0.54 (m, 1H), 0.77-0.88 (m, 1H), 1.58-1.66 (m, 1H), 1.81-1.88 (m, 1H), 1.93-2.00 (m, 1H), 2.12-2.20 (m, 1H), 2.57-2.76 (m, 4H), 2.80 (d, 1H), 5.77 (s, br, 1H), 6.40 (d, 1H), 7.02 (dd, 1H), 7.15 (d, 1H). UPLC-MS (ESI+): [M+H]⁺=324/326 (Br isotope pattern).

### Intermediate B.2

### Preparation of 5-bromo-2-methyl-1,2,2′,3′,5′,6′-hexahydrospiro[indole-3,4′-thiopyran]

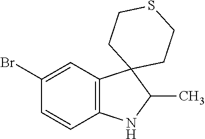 [SMILES: CC1Nc2ccc(Br)cc2C12CCSCC2]

B.2 was prepared in analogy to intermediate B.1 according to GP 3 starting from A.1 and methylmagnesium bromide. UPLC-MS (ESI+): [M+H]⁺=298/300 (Br isotope pattern).

### Intermediate B.3

### Preparation of 5-bromo-2-(prop-2-en-1-yl)-1,2,2′,3′,5′,6′-hexahydrospiro[indole-3,4′-thiopyran]

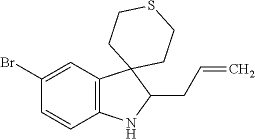 [SMILES: C=CCC1Nc2ccc(Br)cc2C12CCSCC2]

B.3 was prepared in analogy to intermediate B.1 according to GP 3 starting from A.1 and allylmagnesium bromide. ¹H-NMR (300 MHz, CDCl₃): Shift [ppm]=1.80 (m, 1H), 1.96-2.15 (m, 4H), 2.32 (dbr, 1H), 2.65 (m, 1H), 2.70-2.88 (m, 3H), 3.50 (dbr, 1H), 5.62 (dbr, 1H), 5.18 (dbr, 1H), 5.80 (m, 1H), 6.50 (dbr, 1H), 7.14 (dbr, 1H), 7.27 (br. s., 1H). UPLC-MS (ESI+): [M+H]⁺=324/326 (Br isotope pattern).

### Intermediate B.4

### Preparation of 5-bromo-2-vinyl-1,2,2′,3′,5′,6′-hexahydrospiro[indole-3,4′-thiopyran]

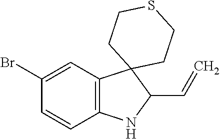 [SMILES: C=CC1Nc2ccc(Br)cc2C12CCSCC2]

B.4 was prepared in analogy to intermediate B.1 according to GP 3 stating from A.1 and vinylmagnesium bromide. ¹H-NMR (300 MHz, CDCl₃): Shift [ppm]=1.80 (m, 1H), 2.00 (m, 4H), 2.55-2.80 (m, 3H), 2.90 (m, 1H), 4.00 (d, 1H), 5.18 (dbr, 1H), 5.30 (dbr, 1H), 5.82 (ddbr, 1H), 6.52 (d, 1H), 7.15 (dbr, 1H), 7.24 (br. s., 1H). UPLC-MS (ESI+): [M+H]⁺=310/312 (Br isotope pattern).

### Intermediate B.5

### Preparation of 5-bromo-2-methyl-1,2,2′,3′,5′,6′-hexahydrospiro[indole-3,4′-pyran]

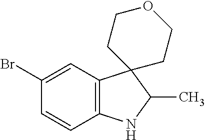 [SMILES: CC1Nc2ccc(Br)cc2C12CCOCC2]

B.5 was prepared in analogy to intermediate B.2 according to GP 3 stating from A.2. UPLC-MS (ESI+): [M+H]⁺=282/284 (Br isotope pattern).

### Intermediate B.6

### Preparation of 5-bromo-2-cyclopropyl-1,2,2′,3′,5′,6′-hexahydrospiro[indole-3,4′-pyran]

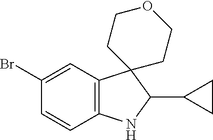 [SMILES: Brc1ccc2c(c1)C1(CCOCC1)C(C1CC1)N2]

According to GP 2 intermediate A.3 (510 mg, 1.67 mmol) and 252 mg (6.67 mmol) sodium borohydride were reacted in 10 mL methanol and purified by preparative HPLC to yield 53 mg (10%) of intermediate B.6. %. ¹H-NMR (300 MHz, DMSO-d6): Shift [ppm]=1.07-1.21 (m, 1H), 1.48-1.92 (m, 7H), 2.02-2.12 (m, 1H), 3.00-3.09 (m, 1H), 3.42 (dt, 1H), 3.67 (dt, 1H), 3.75-3.89 (m, 3H), 6.49 (d, 1H), 7.15-7.20 (m, 2H). UPLC-MS (ESI+): [M+H]⁺=308/310 (Br isotope pattern).

### Intermediate C.1

### Preparation of 5-bromo-2-cyclopropyl-1-[(4-fluorophenyl)sulfonyl]-1,2,2′,3′,5′,6′-hexahydrospiro[indole-3,4′-thiopyran]

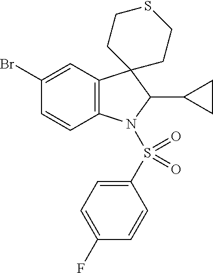 [SMILES: O=S(=O)(c1ccc(F)cc1)N1c2ccc(Br)cc2C2(CCSCC2)C1C1CC1]

According to GP 4.1 indoline B.1 (8.88 mmol) was reacted with 5 eq. triethylamine and 3 eq. 4-fluorobenzenesulfonyl chloride (CAS No. [349-88-2]) in 180 mL 1,2-dichloroethane at 80° C. for 18 h, leading to 80% conversion (by LCMS). Further 3 eq. triethylamine and 2 eq. 4-fluorobenzenesulfonyl chloride were added and stirred for further 24 h at 80° C. to drive the reaction to completion. Isolated yield: 52%. ¹H-NMR (300 MHz, DMSO-d6): Shift [ppm]=0.19 (d, 1H), 0.30-0.45 (m, 2H), 0.51-0.61 (m, 1H), 0.66-0.75 (m, 1H), 0.88-1.02 (m, 2H), 1.94 (d, 1H), 2.03-2.13 (m, 1H), 2.23-2.31 (m, 1H), 2.56 (d, 1H), 2.69-2.86 (m, 2H), 3.98 (d, 1H), 7.33-7.42 (m, 5H), 7.80-7.84 (m, 2H). UPLC-MS (ESI+): [M+H]⁺=482/484 (Br isotope pattern).

### Intermediate C.2

### Preparation of 5-bromo-1-[(4-fluorophenyl)sulfonyl]-2-methyl-1,2,2′,3′,5′,6′-hexahydrospiro[indole-3,4′-thiopyran]

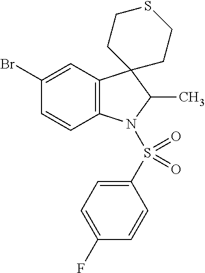 [SMILES: CC1N(S(=O)(=O)c2ccc(F)cc2)c2ccc(Br)cc2C12CCSCC2]

C.2 was prepared in analogy to intermediate C.1 according to GP 4.1 starting from B.2. UPLC-MS (ESI+): [M+H]⁺=456/458 (Br isotope pattern).

### Intermediate C.3

### Preparation of methyl 3-{[5-bromo-2-(prop-2-en-1-yl)-1,2,2′,3′,5′,6′-hexahydrospiro[indole-3,4′-thiopyran]-1-yl]sulfonyl}benzoate

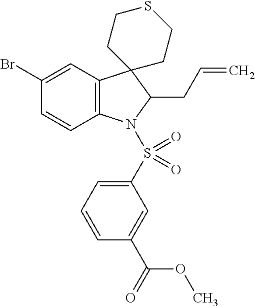 [SMILES: C=CCC1N(S(=O)(=O)c2cccc(C(=O)OC)c2)c2ccc(Br)cc2C12CCSCC2]

C.3 was prepared according to GP 4.2 starting from B.3 and methyl 3-(chlorosulfonyl)benzoate (CAS No. [63555-50-0]). ¹H-NMR (400 MHz, CDCl₃): Shift [ppm]=0.48 (dbr, 1H), 1.10 (tbr, 1H), 2.06 (m, 2H), 2.22 (dbr, 1H), 2.32 (m, 1H), 2.61 (m, 2H), 2.70 (qbr, 1H), 3.96 (s, 3H), 4.38 (m, 1H), 5.02-5.12 (m, 2H), 5.78 (m, 1H), 7.11 (s, 1H), 7.37 (dbr, 1H), 7.53 (m, 2H), 7.95 (dbr, 1H), 8.23 (dbr, 1H), 8.49 (br. s.). UPLC-MS (ESI+): [M+H]⁺=522/524 (Br isotope pattern).

### Intermediate C.4

### Preparation of methyl 3-[(5-bromo-2-vinyl-1,2,2′,3′,5′,6′-hexahydrospiro[indole-3,4′-thiopyran]-1-yl)sulfonyl]benzoate

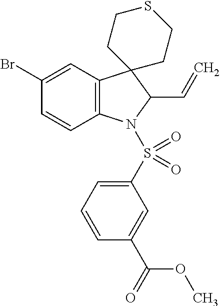 [SMILES: C=CC1N(S(=O)(=O)c2cccc(C(=O)OC)c2)c2ccc(Br)cc2C12CCSCC2]

C.4 was prepared in analogy to intermediate C.3 according to GP 4.1 starting from B.4 and methyl 3-(chlorosulfonyl)benzoate (CAS No. [63555-50-0]). ¹H-NMR (300 MHz, CDCl₃): Shift [ppm]=0.98 (m, 1H), 1.36 (m, 1H), 2.03 (m, 2H), 2.21 (dbr, 1H), 2.50 (dbr, 1H), 2.60-2.90 (m, 3H), 3.96 (s, 3H) 4.70 (d, 1H), 5.29 (dbr, 1H), 5.50 (dbr, 1H), 5.68 (ddbr, 1H), 1.17 (s, 1H), 7.35 (dbr, 1H), 7.46 (dbr, 1H), 7.53 (m, 1H), 7.98 (dbr, 1H), 8.22 (dbr, 1H), 8.51 (br. s., 1H). UPLC-MS (ESI+): [M+H]⁺=508/510 (Br isotope pattern).

### Intermediate C.5

### Preparation of 5-bromo-1-[(4-fluorophenyl)sulfonyl]-2-methyl-1,2,2′,3′,5′,6′-hexahydrospiro[indole-3,4′-pyran]

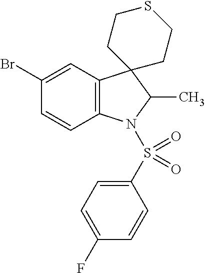 [SMILES: CC1N(S(=O)(=O)c2ccc(F)cc2)c2ccc(Br)cc2C12CCSCC2]

C.5 was prepared in analogy to intermediate C.2 according to GP 4.1 starting from B.5. ¹H-NMR (400 MHz, DMSO-d6): Shift [ppm]=−0.13 (d, 1H), 1.03 (dt, 1H), 1.22 (d, 3H), 1.59 (dd, 1H), 2.00 (dt, 1H), 3.30-3.37 (m, 2H), 3.43 (dt, 1H), 3.74-3.81 (m, 1H), 4.45 (q, 1H), 7.35-7.43 (m, 5H), 7.84-7.89 (m, 2H). UPLC-MS (ESI+): [M+H]⁺=440/442 (Br isotope pattern).

### Intermediate C.6

### Preparation of 5-bromo-2-cyclopropyl-1-[(3-methoxyphenyl)sulfonyl]-1,2,2′,3′,5′,6′-hexahydrospiro[indole-3,4′-thiopyran]

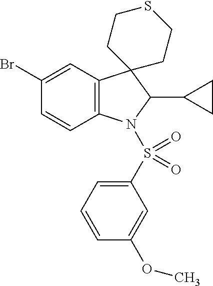 [SMILES: COc1cccc(S(=O)(=O)N2c3ccc(Br)cc3C3(CCSCC3)C2C2CC2)c1]

C.6 was prepared in analogy to intermediate C.1 according to GP 4.1 starting from B.1 and 3-methoxybenzenesulfonyl chloride (CAS No. [10130-74-2]). UPLC-MS (ESI+): [M+H]⁺=494/496 (Br isotope pattern).

### Intermediate C.7

### Preparation of 4-[(5-bromo-2-cyclopropyl-2′,3′,5′,6′-tetrahydrospiro[indole-3,4′-thiopyran]-1 (2H)-yl)sulfonyl]benzonitrile

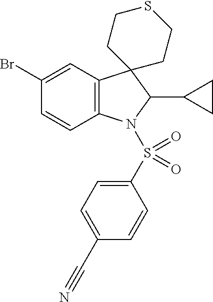 [SMILES: N#Cc1ccc(S(=O)(=O)N2c3ccc(Br)cc3C3(CCSCC3)C2C2CC2)cc1]

C.7 was prepared according to GP 4.2 starting from B.1 and 4-cyanobenzenesulfonyl chloride (CAS No. [60958-06-7]). UPLC-MS (ESI+): [M+H]⁺=489/491 (Br isotope pattern).

### Intermediate C.8

### Preparation of 3-[(5-bromo-2-cyclopropyl-2′,3′,5′,6′-tetrahydrospiro[indole-3,4′-thiopyran]-1(2H)-yl)sulfonyl]benzonitrile

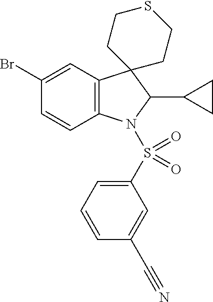 [SMILES: N#Cc1cccc(S(=O)(=O)N2c3ccc(Br)cc3C3(CCSCC3)C2C2CC2)c1]

C.8 was prepared according to GP 4.2 starting from B.1 and 3-cyanobenzenesulfonyl chloride (CAS No. [56542-67-7]). UPLC-MS (ESI+): [M+H]⁺=489/491 (Br isotope pattern).

### Intermediate C.9

### Preparation of 5-bromo-2-cyclopropyl-1-{[3-(trifluoromethoxy)phenyl]sulfonyl}-1,2,2′,3′,5′,6′-hexahydrospiro[indole-3,4′-thiopyran]

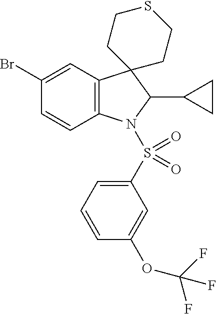 [SMILES: O=S(=O)(c1cccc(OC(F)(F)F)c1)N1c2ccc(Br)cc2C2(CCSCC2)C1C1CC1]

C.9 was prepared according to GP 4.2 starting from B.1 and 3-trifluoromethoxybenzene-sulfonyl chloride (CAS No. [220227-84-9]). UPLC-MS (ESI+): [M+H]⁺=548/550 (Br isotope pattern).

### Intermediate C.10

### Preparation of 5-bromo-2-cyclopropyl-1-{[3-(difluoromethoxy)phenyl]sulfonyl}-1,2,2′,3′,5′,6′-hexahydrospiro[indole-3,4′-thiopyran]

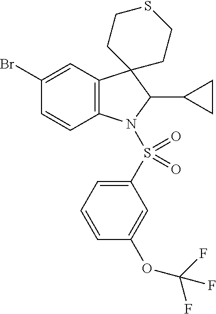 [SMILES: O=S(=O)(c1cccc(OC(F)(F)F)c1)N1c2ccc(Br)cc2C2(CCSCC2)C1C1CC1]

C.10 was prepared according to GP 4.2 starting from B.1 and 3-difluoromethoxybenzene-sulfonyl chloride (CAS No. [351003-38-8]). UPLC-MS (ESI+): [M+H]⁺=530/532.

### Intermediate C.11

### Preparation of 5-bromo-2-cyclopropyl-1-{[4-(difluoromethoxy)phenyl]sulfonyl}-1,2,2′,3′,5′,6′-hexahydrospiro[indole-3,4′-thiopyran]

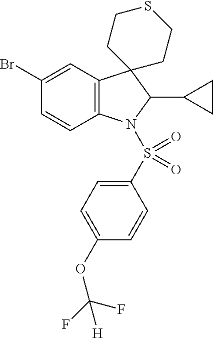 [SMILES: O=S(=O)(c1ccc(OC(F)F)cc1)N1c2ccc(Br)cc2C2(CCSCC2)C1C1CC1]

C.11 was prepared according to GP 4.2 starting from B.1 and 4-difluoromethoxybenzene-sulfonyl chloride (CAS No. [351003-34-4]). UPLC-MS (ESI+): [M+H]⁺=530/532 (Br isotope pattern).

### Intermediate C.12

### Preparation of 4-[(5-bromo-2-cyclopropyl-2′,3′,5′,6′-tetrahydrospiro[indole-3,4′-thiopyran]-1(2H)-yl)sulfonyl]benzamide

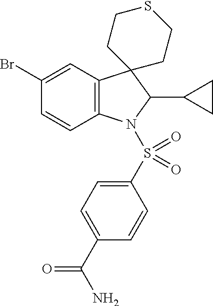 [SMILES: NC(=O)c1ccc(S(=O)(=O)N2c3ccc(Br)cc3C3(CCSCC3)C2C2CC2)cc1]

C.12 was prepared according to GP 4.2 starting from B.1 and 4-carbamoylbenzenesulfonyl-chloride (CAS No. [885526-86-3]). ¹H-NMR (300 MHz, DMSO-d6): Shift [ppm]=0.09-0.13 (m, 1H), 0.34-0.51 (m, 2H), 0.56-0.65 (m, 1H), 0.72-0.81 (m, 1H), 0.90-1.00 (m, 2H), 1.87-1.92 (m, 1H), 2.06-2.16 (m, 1H), 2.29-2.34 (m, 1H), 2.56-2.61 (m, 1H), 2.76-2.89 (m, 2H), 3.99-4.06 (m, 1H), 7.39-7.48 (m, 3H), 7.62 (br. s., 1H), 7.84-7.87 (m, 2H), 7.94-7.97 (m, 2H), 8.14 (br. s., 1H). UPLC-MS (ESI+): [M+H]⁺=507/509 (Br isotope pattern).

### Intermediate C.13 Preparation of 5-bromo-1-[(4-fluorophenyl)sulfonyl]-2-(prop-2-en-1-yl)-1,2,2′,3′,5′,6′-hexahydrospiro[indole-3,4′-thiopyran]

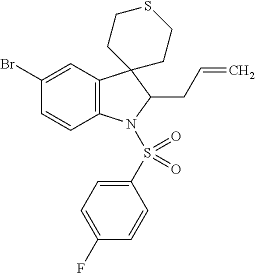 [SMILES: C=CCC1N(S(=O)(=O)c2ccc(F)cc2)c2ccc(Br)cc2C12CCSCC2]

C.13 was prepared according to GP 4.2 starting from B.3 and 4-fluorobenzenesulfonyl chloride (CAS No. [349-88-2]). ¹H-NMR (400 MHz, CDCl₃): Shift [ppm]=0.52 (dbr, 1H), 1.13 m (1H), 2.02-2.18 (m, 2H), 2.21 (dbr, 1H), 2.32 (m, 1H), 2.55-2.68 (m, 3H), 2.75 (m, 1H), 4.29 (m, 1H), 5.00-5.10 (m, 2H), 5.80 (m, 1H), 7.13 (m, 3H), 7.38 (dbr, 1H), 7.52 (d, 1H), 7.82 (m, 2H). UPLC-MS (ESI+): [M+H]⁺=482/484 (Br isotope pattern).

### Intermediate C.14

### Preparation of methyl 3-[(5-bromo-2-cyclopropyl-2′,3′,5′,6′-tetrahydrospiro[indole-3,4′-thiopyran]-1 (2H)-yl)sulfonyl]benzoate

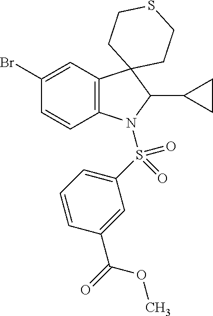 [SMILES: COC(=O)c1cccc(S(=O)(=O)N2c3ccc(Br)cc3C3(CCSCC3)C2C2CC2)c1]

C.14 was prepared according to GP 4.2 starting from B.1 and methyl 3-(chlorosulfonyl)benzoate (CAS No. [63555-50-0]). ¹H-NMR (300 MHz, DMSO-d6): Shift [ppm]=0.13-0.24 (m, 1H), 0.33-0.52 (m, 1H), 0.53-0.66 (m, 1H), 0.75-0.88 (m, 1H), 3.87 (s, 3H), 4.08 (d, 1H), 7.39-7.46 (m, 3H), 7.72 (tr, 1H), 8.03-8.10 (m, 1H), 8.16-8.26 (m, 2H). UPLC-MS (ESI+): [M+H]⁺=522/524 (Br isotope pattern).

### Intermediate D.1

### Preparation of 5-bromo-2-cyclopropyl-1-[(4-fluorophenyl)sulfonyl]-1,2,2′,3′,5′,6′-hexahydrospiro[indole-3,4′-thiopyran]1′,1′-dioxide

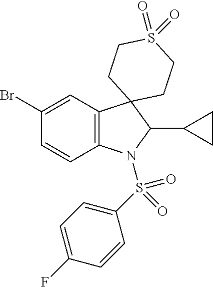 [SMILES: O=S1(=O)CCC2(CC1)c1cc(Br)ccc1N(S(=O)(=O)c1ccc(F)cc1)C2C1CC1]

According to GP 5.2 8.84 g (18.3 mmol) of intermediate C.1 were oxidized with 13.8 g (8 eq.) urea hydrogen peroxide/23 g (6 eq.) trifluoroacetic anhydride to yield 9.25 g (98%) of the desired sulfone. ¹H-NMR (300 MHz, DMSO-d6): Shift [ppm]=0.13-0.22 (m, 1H), 0.32-0.48 (m, 2H), 0.50-0.60 (m, 1H), 0.74-0.83 (m, 1H), 0.89-1.01 (m, 1H), 1.41 (dt, 1H), 2.34-2.58 (m, 3H), 3.09-3.17 (m, 2H), 3.56 (dt, 1H), 4.26 (d, 1H), 7.34-7.47 (m, 5H), 7.80-7.88 (m, 2H). UPLC-MS (ESI+): [M+H]⁺=514/516 (Br isotope pattern).

Alternatively, 8 mmol of intermediate C.1 (3.86 g) were oxidized according to GP 5.1 with 3 eq (4.19 g) of 3-chloroperoxybenzoic acid for 4 h at 0° C. to yield 2.3 g (56%) of the desired sulfone (identical by UPLC).

### Intermediate D.2

### Preparation of 5-bromo-1-[(4-fluorophenyl)sulfonyl]-2-methyl-1,2,2′,3′,5′,6′-hexahydrospiro[indole-3,4′-thiopyran]1′,1′-dioxide

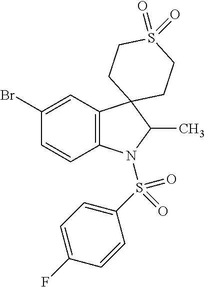 [SMILES: CC1N(S(=O)(=O)c2ccc(F)cc2)c2ccc(Br)cc2C12CCS(=O)(=O)CC2]

D.2 was prepared in analogy to intermediate D.1 according to GP 5.1 starting from C.2. UPLC-MS (ESI+): [M+H]⁺=488/490 (Br isotope pattern).

### Intermediate D.3

### Preparation of methyl 3-{[5-bromo-1′,1′-dioxido-2-(prop-2-en-1-yl)-1,2,2′,3′,5′,6′-hexahydrospiro[indole-3,4′-thiopyran]-1-yl]sulfonyl}benzoate

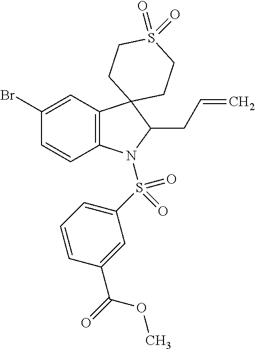 [SMILES: C=CCC1N(S(=O)(=O)c2cccc(C(=O)OC)c2)c2ccc(Br)cc2C12CCS(=O)(=O)CC2]

D.3 was prepared in a modification to GP 5.3 starting from C.3. Deviating from GP 5.3 the reaction mixture was cooled to −20° C. during the addition of Oxone® and it was stirred at −20° C. for 7 hours after the addition was completed. Afterward, it was worked-up as described in GP 5.3. ¹H-NMR (300 MHz, CDCl₃): Shift [ppm]=0.74 (m, 1H), 0.88 (m, 1H), 1.30 (m, 1H), 1.68 (tbr, 1H), 2.30-2.42 (m, 2H), 2.50-2.68 (m, 3H), 2.92-3.20 (m, 3H), 3.98 (s, 3H), 4.40 (tbr 1H), 5.02-5.15 (m, 2H), 5.68-5.85 (m, 1H), 7.18 (br. s., 1H), 7.42 (dbr, 1H), 7.50 (dbr, 1H), 7.58 (t, 1H), 7.97 (dbr, 1H), 8.23 (dbr, 1H), 8.49 (br. s., 1H). UPLC-MS (ESI+): [M+H]⁺=554/556 (Br isotope pattern).

### Intermediate D.4

### Preparation of methyl 3-[(5-bromo-1′,1′-dioxido-2-vinyl-1,2,2′,3′,5′,6′-hexahydrospiro[indole-3,4′-thiopyran]-1-yl)sulfonyl]benzoate

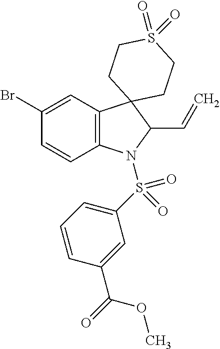 [SMILES: C=CC1N(S(=O)(=O)c2cccc(C(=O)OC)c2)c2ccc(Br)cc2C12CCS(=O)(=O)CC2]

D.4 was prepared according to GP 5.3 starting from C.4. ¹H-NMR (400 MHz, CDCl₃): Shift [ppm]=1.30 (m, 1H), 1.97 (dt, 1H), 2.15 (dbr, 1H), 2.50-2.65 (m, 1H), 2.82 (br. s., 1H), 2.81 (dbr, 1H), 3.02 (m, 2H), 3.15 (dt, 1H), 3.97 (s, 3H), 4.74 (d, 2H), 4.33 (d, 1H), 5.55 (d, 1H), 5.67 (ddbr, 1H), 7.25 (dbr, 1H), 7.38-7.48 (m, 2H), 7.60 (dd, 1H), 8.02 (dbr, 1H), 8.26 (dbr, 1H), 8.51 (br. s., 1H). UPLC-MS (ESI+): [M+H]⁺=540/542 (Br isotope pattern).

### Intermediate D.5

### Preparation of 5-bromo-2-cyclopropyl-1-[(3-methoxyphenyl)sulfonyl]-1,2,2′,3′,5′,6′-hexahydrospiro[indole-3,4′-thiopyran]1′,1′-dioxide

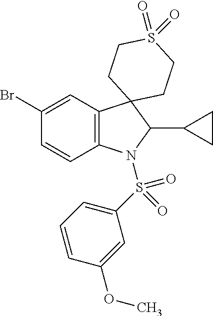 [SMILES: COc1cccc(S(=O)(=O)N2c3ccc(Br)cc3C3(CCS(=O)(=O)CC3)C2C2CC2)c1]

D.5 was prepared in analogy to intermediate D.1 according to GP 5.2 starting from C.6. UPLC-MS (ESI+): [M+H]⁺=526/528 (Br isotope pattern).

### Intermediate D.6

### Preparation of 4-[(5-bromo-2-cyclopropyl-1′,1′-dioxido-2′,3′,5′,6′-tetrahydrospiro[indole-3,4′-thiopyran]-1 (2H)-yl)sulfonyl]benzonitrile

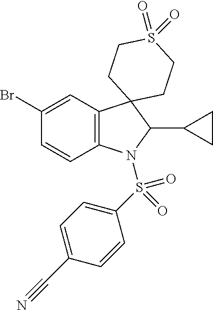 [SMILES: N#Cc1ccc(S(=O)(=O)N2c3ccc(Br)cc3C3(CCS(=O)(=O)CC3)C2C2CC2)cc1]

D.6 was prepared in analogy to intermediate D.1 according to GP 5.2 starting from C.7. ¹H-NMR (300 MHz, DMSO-d6): Shift [ppm]=0.08 (d, 1H), 0.33-0.43 (m, 1H), 0.44-0.61 (m, 2H), 0.75-0.84 (m, 1H), 0.90-1.01 (m, 1H), 1.33-1.44 (m, 1H), 2.33-2.43 (m, 1H), 2.51-2.67 (m, 2H), 3.04-3.19 (m, 2H), 3.48-3.60 (m, 1H), 4.27 (d, 1H), 7.39-7.49 (m, 3H), 7.93 (d, 2H), 8.01 (d, 2H). UPLC-MS (ESI+): [M+H]⁺=522/524 (Br isotope pattern).

### Intermediate D.7

### Preparation of 3-[(5-bromo-2-cyclopropyl-1′,1′-dioxido-2′,3′,5′,6′-tetrahydrospiro[indole-3,4′-thiopyran]-1 (2H)-yl)sulfonyl]benzonitrile

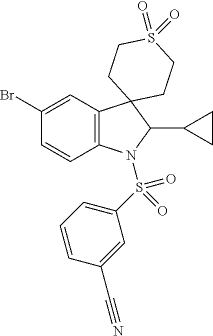 [SMILES: N#Cc1cccc(S(=O)(=O)N2c3ccc(Br)cc3C3(CCS(=O)(=O)CC3)C2C2CC2)c1]

D.7 was prepared in analogy to intermediate D.1 according to GP 5.2 starting from C.8. UPLC-MS (ESI+): [M+H]⁺=522/524 (Br isotope pattern).

### Intermediate D.8

### Preparation of 5-bromo-2-cyclopropyl-1-{[3-(trifluoromethoxy)phenyl]sulfonyl}-1,2,2′,3′,5′,6′-hexahydrospiro[indole-3,4′-thiopyran]1′,1′-dioxide

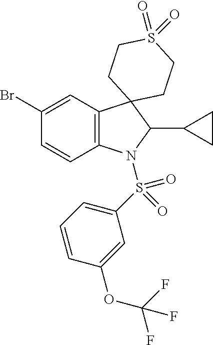 [SMILES: O=S1(=O)CCC2(CC1)c1cc(Br)ccc1N(S(=O)(=O)c1cccc(OC(F)(F)F)c1)C2C1CC1]

D.8 was prepared in analogy to intermediate D.1 according to GP 5.2 starting from C.9. UPLC-MS (ESI+): [M+H]⁺=580/582 (Br isotope pattern).

### Intermediate D.9

### Preparation of 5-bromo-2-cyclopropyl-1-{[3-(difluoromethoxy)phenyl]sulfonyl}-1,2,2′,3′,5′,6′-hexahydrospiro[indole-3,4′-thiopyran]1′,1′-dioxide

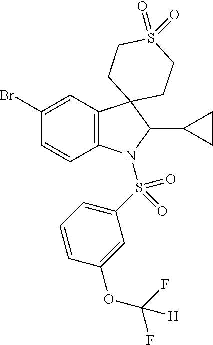 [SMILES: O=S1(=O)CCC2(CC1)c1cc(Br)ccc1N(S(=O)(=O)c1cccc(OC(F)F)c1)C2C1CC1]

D.9 was prepared in analogy to intermediate D.1 according to GP 5.2 starting from C.10. UPLC-MS (ESI+): [M+H]⁺=562/564 (Br isotope pattern).

### Intermediate D.10

### Preparation of 5-bromo-2-cyclopropyl-1-{[4-(difluoromethoxy)phenyl]sulfonyl}-1,2,2′,3′,5′,6′-hexahydrospiro[indole-3,4′-thiopyran]1′,1′-dioxide

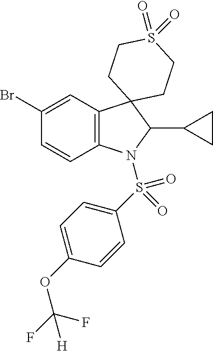 [SMILES: O=S1(=O)CCC2(CC1)c1cc(Br)ccc1N(S(=O)(=O)c1ccc(OC(F)F)cc1)C2C1CC1]

D.10 was prepared in analogy to intermediate D.1 according to GP 5.2 starting from C.11. UPLC-MS (ESI+): [M+H]⁺=562/564 (Br isotope pattern).

### Intermediate D.11

### Preparation of 4-[(5-bromo-2-cyclopropyl-1′,1′-dioxido-2′,3′,5′,6′-tetrahydrospiro[indole-3,4′-thiopyran]-1 (2H)-yl)sulfonyl]benzamide

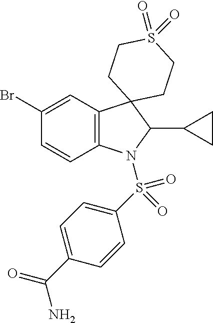 [SMILES: NC(=O)c1ccc(S(=O)(=O)N2c3ccc(Br)cc3C3(CCS(=O)(=O)CC3)C2C2CC2)cc1]

D.11 was prepared in a modification to GP 5.2 starting from C.12. Deviating from GP 5.2 the reaction mixture was filtered upon completion and the obtained residue washed with acetonitrile to get a first crop of product. The filtrate was worked-up as described in GP 5.2 to get a second crop. Both materials were combined and taken to the next step without further purification. ¹H-NMR (400 MHz, DMSO-d6): Shift [ppm]=0.08-0.13 (m, 1H), 0.38-0.45 (m, 1H), 0.48-0.54 (m, 1H), 0.56-0.63 (m, 1H), 0.82-0.88 (m, 1H), 0.95-1.03 (m, 1H), 1.39 (dt, 1H), 2.39-2.56 (m, 3H), 3.16-3.18 (m, 2H), 3.60 (dt, 1H), 4.32 (d, 1H), 7.46-7.48 (m, 3H), 7.60 (br. s., 1H), 7.86-7.88 (m, 2H), 7.93-7.96 (m, 2H), 8.12 (br. s., 1H). UPLC-MS (ESI+): [M+H]⁺=539/541 (Br isotope pattern).

### Intermediate D.12

### Preparation of 5-bromo-1-[(4-fluorophenyl)sulfonyl]-2-(prop-2-en-1-yl)-1,2,2′,3′,5′,6′-hexahydrospiro[indole-3,4′-thiopyran]1′,1′-dioxide

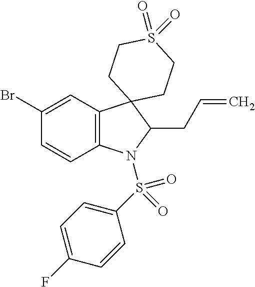 [SMILES: C=CCC1N(S(=O)(=O)c2ccc(F)cc2)c2ccc(Br)cc2C12CCS(=O)(=O)CC2]

D.12 was prepared in a modification to GP 5.3 starting from C.13. Deviating from GP 5.3 the reaction mixture was cooled to −20° C. during the addition of Oxone® and it was stirred at −20° C. for 7 hours after the addition was completed. Afterward, it was worked-up as described in GP 5.3. ¹H-NMR (300 MHz, CDCl₃): Shift [ppm]=0.72 (dbr, 1H), 1.68 (tbr, 1H), 2.35 (m, 2H), 2.88-3.20 (m, 3H), 4.30 (t, 1H), 5.05-5.17 (m, 2H), 5.75 (m, 1H), 7.12-7.25 (3H), 7.41 (d, 1H), 7.51 (d, 1H), 7.85 (m, 2H). HPLC-MS (ESI+): [M]⁺=514/516 (Br isotope pattern).

### Intermediate D.13

### Preparation of methyl 3-[(5-bromo-2-cyclopropyl-1′,1′-dioxido-2′,3′,5′,6′-tetrahydrospiro[indole-3,4′-thiopyran]-1 (2H)-yl)sulfonyl]benzoate

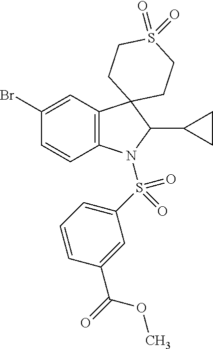 [SMILES: COC(=O)c1cccc(S(=O)(=O)N2c3ccc(Br)cc3C3(CCS(=O)(=O)CC3)C2C2CC2)c1]

D.13 was prepared in analogy to intermediate D.1 according to GP 5.2 starting from C.14 HPLC-MS (ESI+): [M]⁺=554/556 (Br isotope pattern).

### Intermediate E.1

### Preparation of methyl 2-cyclopropyl-1-[(4-fluorophenyl)sulfonyl]-1,2,2′,3′,5′,6′-hexahydrospiro[indole-3,4′-thiopyran]-5-carboxylate 1′,1′-dioxide

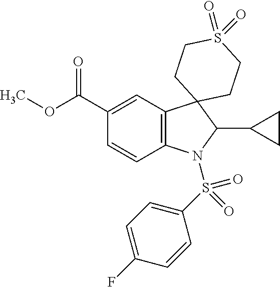 [SMILES: COC(=O)c1ccc2c(c1)C1(CCS(=O)(=O)CC1)C(C1CC1)N2S(=O)(=O)c1ccc(F)cc1]

According to GP 6 4.2 mmol of intermediate D.1 were carbonylated in a mixture of 120 mL methanol, 12 mL DMSO and 1.4 mL triethylamine (10.5 mmol) in the presence of 600 mg trans-bis(triphenylphosphine) palladium(II) dichloride (0.84 mmol). A carbon monoxide pressure of 8.59 bar was applied at 20° C., then the autoclave was heated to 100° C. internal temperature to reach a pressure of 12.2 bar. The reaction was complete after 22 h. Yield: 1.80 g of the desired methyl ester (82%). UPLC-MS (ESI+): [M+H]⁺=494.

### Intermediate E.2

### Preparation of methyl 1-[(4-fluorophenyl)sulfonyl]-2-methyl-1,2,2′,3′,5′,6′-hexahydrospiro[indole-3,4′-thiopyran]-5-carboxylate 1′,1′-dioxide

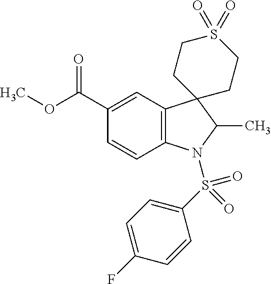

E.2 was prepared in analogy to intermediate E.1 according to GP 6 starting from D.2. UPLC-MS (ESI+): [M+H]⁺=467.

### Intermediate E.3

### Preparation of methyl 1-[(4-fluorophenyl)sulfonyl]-2-methyl-1,2,2′,3′,5′,6′-hexahydrospiro[indole-3,4′-pyran]-5-carboxylate

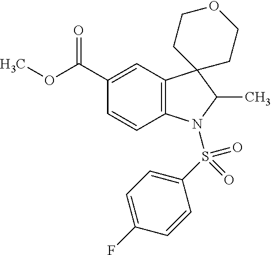

E.3 was prepared in analogy to intermediate E.2 according to GP 6 starting from C.5. ¹H-NMR (400 MHz, DMSO-d6): Shift [ppm]=0.01 (d, 1H), 1.03 (dt, 1H), 1.24 (d, 3H), 1.66 (d, 1H), 2.02 (dt, 1H), 3.33-3.39 (m, 2H), 3.47 (dt, 1H), 3.78 (s, 3H), 3.79-3.84 (m, 1H), 4.54 (q, 1H), 7.36-7.41 (m, 2H), 7.59 (d, 1H), 7.68 (d, 1H), 7.87 (dd, 1H), 7.89-7.93 (m, 2H). UPLC-MS (ESI+): [M+H]⁺=420.

### Intermediate E.4

### Preparation of methyl 2-cyclopropyl-1-[(3-methoxyphenyl)sulfonyl]-1,2,2′,3′,5′,6′-hexahydrospiro[indole-3,4′-thiopyran]-5-carboxylate 1′,1′-dioxide

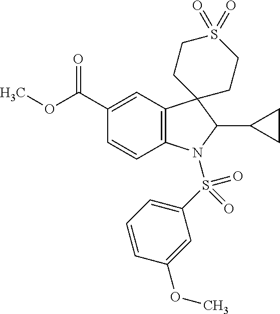 [SMILES: COC(=O)c1ccc2c(c1)C1(CCS(=O)(=O)CC1)C(C1CC1)N2S(=O)(=O)c1cccc(OC)c1]

E.4 was prepared in analogy to intermediate E.1 according to GP 6 starting from D.5. UPLC-MS (ESI+): [M+H]⁺=506.

### Intermediate E.5

### Preparation of methyl 2-cyclopropyl-1-{[3-(trifluoromethoxy)phenyl]sulfonyl}-1,2,2′,3′,5′,6′-hexahydrospiro[indole-3,4′-thiopyran]-5-carboxylate 1′,1′-dioxide

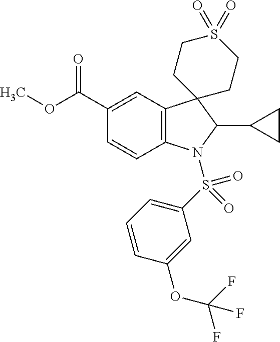 [SMILES: COC(=O)c1ccc2c(c1)C1(CCS(=O)(=O)CC1)C(C1CC1)N2S(=O)(=O)c1cccc(OC(F)(F)F)c1]

E.5 was prepared in analogy to intermediate E.1 according to GP 6 starting from D.8. ¹H-NMR (400 MHz, DMSO-d6): Shift [ppm]=0.19 (d, 1H), 0.32-0.62 (m, 3H), 0.76-0.85 (m, 1H), 0.89-1.02 (m, 1H), 1.40 (dt, 1H), 3.62 (dt, 1H), 3.79 (s, 3H), 4.35 (d, 1H), 7.60-7.96 (m, 7H). UPLC-MS (ESI+): [M+H]⁺=560.

### Intermediate E.6

### Preparation of methyl 2-cyclopropyl-1-{[3-(difluoromethoxy)phenyl]sulfonyl}-1,2,2′,3′,5′,6′-hexahydrospiro[indole-3,4′-thiopyran]-5-carboxylate 1′,1′-dioxide

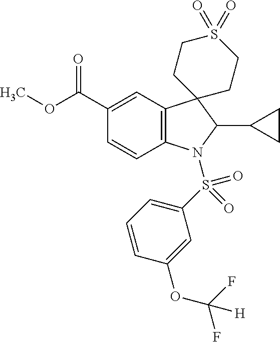 [SMILES: COC(=O)c1ccc2c(c1)C1(CCS(=O)(=O)CC1)C(C1CC1)N2S(=O)(=O)c1cccc(OC(F)F)c1]

E.6 was prepared in analogy to intermediate E.1 according to GP 6 starting from D.9. UPLC-MS (ESI+): [M+H]⁺=542.

### Intermediate E.7

### Preparation of methyl 2-cyclopropyl-1-{[4-(difluoromethoxy)phenyl]sulfonyl}-1,2,2′,3′,5′,6′-hexahydrospiro[indole-3,4′-thiopyran]-5-carboxylate 1′,1′-dioxide

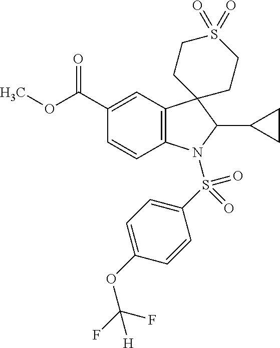 [SMILES: COC(=O)c1ccc2c(c1)C1(CCS(=O)(=O)CC1)C(C1CC1)N2S(=O)(=O)c1ccc(OC(F)F)cc1]

E.7 was prepared in analogy to intermediate E.1 according to GP 6 starting from D.10. UPLC-MS (ESI+): [M+H]⁺=542.

### Intermediate E.8

### Preparation of methyl 1-[(4-fluorophenyl)sulfonyl]-2-(prop-2-en-1-yl)-1,2,2′,3′,5′,6′-hexahydrospiro[indole-3,4′-thiopyran]-5-carboxylate 1′,1′-dioxide

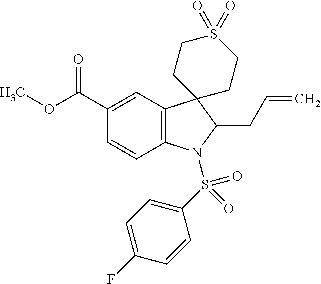 [SMILES: C=CCC1N(S(=O)(=O)c2ccc(F)cc2)c2ccc(C(=O)OC)cc2C12CCS(=O)(=O)CC2]

E.8 was prepared in analogy to intermediate E.1 according to GP 6 starting from D.12. ¹H-NMR (400 MHz, CDCl₃): Shift [ppm]=0.81 (dbr, 1H), 1.72 (tbr, 1H), 2.49 (m, 2H), 2.60-2.77 (m, 3H), 2.90-3.15 (m, 2H), 3.19 (m, 1H), 3.91 (s, 3H), 4.38 (t, 1H), 5.00-5.12 (m, 2H), 5.70 (m, 1H), 7.18 (t, 2H), 7.68 (d, 1H), 7.78 (s, 1H), 7.87 (m, 2H), 8.04 (d, 1H). UPLC-MS (ESI+): [M+H]⁺=494.

### Intermediate F.1

### Preparation of 2-cyclopropyl-1-[(4-fluorophenyl)sulfonyl]-1,2,2′,3′,5′,6′-hexahydrospiro[indole-3,4′-thiopyran]-5-carboxylic acid 1′,1′-dioxide

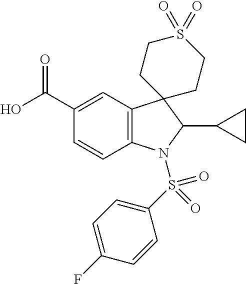 [SMILES: O=C(O)c1ccc2c(c1)C1(CCS(=O)(=O)CC1)C(C1CC1)N2S(=O)(=O)c1ccc(F)cc1]

According to GP 7 1.90 g of the intermediate E.1 were hydrolyzed in 130 mL of a 1:1 mixture of THF and 2M aqueous lithium hydroxide solution to yield 1.50 g (77%) of the desired carboxylic acid. UPLC-MS (ESI−): [M−H]⁻=478.

### Intermediate F.2

### Preparation of 1-[(4-fluorophenyl)sulfonyl]-2-methyl-1,2,2′,3′,5′,6′-hexahydrospiro[indole-3,4′-thiopyran]-5-carboxylic acid 1′,1′-dioxide

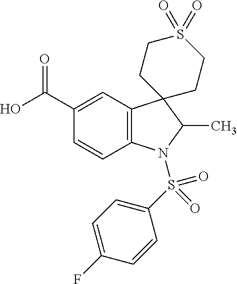 [SMILES: CC1N(S(=O)(=O)c2ccc(F)cc2)c2ccc(C(=O)O)cc2C12CCS(=O)(=O)CC2]

F.2 was prepared in analogy to intermediate F.1 according to GP 7 starting from E.2. UPLC-MS (ESI−): [M−H]⁻=452.

### Intermediate F.3

### Preparation of 1-[(4-fluorophenyl)sulfonyl]-2-methyl-1,2,2′,3′,5′,6′-hexahydrospiro[indole-3,4′-pyran]-5-carboxylic acid

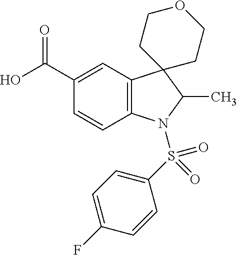 [SMILES: CC1N(S(=O)(=O)c2ccc(F)cc2)c2ccc(C(=O)O)cc2C12CCOCC2]

F.3 was prepared in analogy to intermediate F.2 according to GP 7 starting from E.3. ¹H-NMR (400 MHz, DMSO-d6): Shift [ppm]=0.01 (d, 1H), 1.03 (dt, 1H), 1.24 (d, 3H), 1.66 (d, 1H), 2.00 (dt, 1H), 3.33-3.39 (m, 2H), 3.47 (dt, 1H), 3.78-3.84 (m, 1H), 4.53 (q, 1H), 7.36-7.41 (m, 2H), 7.56 (d, 1H), 7.65 (d, 1H), 7.87 (dd, 1H), 7.89-7.93 (m, 2H). UPLC-MS (ESI−): [M−H]⁻=404; UPLC-MS (ESI+): [M+H]⁺=406.

### Intermediate F.4

### Preparation of 2-cyclopropyl-1-[(3-methoxyphenyl)sulfonyl]-1,2,2′,3′,5′,6′-hexahydrospiro[indole-3,4′-thiopyran]-5-carboxylic acid 1′,1′-dioxide

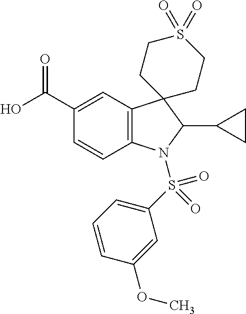 [SMILES: COc1cccc(S(=O)(=O)N2c3ccc(C(=O)O)cc3C3(CCS(=O)(=O)CC3)C2C2CC2)c1]

F.4 was prepared in analogy to intermediate F.1 according to GP 7 starting from E.4. UPLC-MS (ESI−): [M−H]⁻=490.

### Intermediate F.5

### Preparation of 2-cyclopropyl-1-{[3-(trifluoromethoxy)phenyl]sulfonyl}-1,2,2′,3′,5′,6′-hexahydrospiro[indole-3,4′-thiopyran]-5-carboxylic acid 1′,1′-dioxide

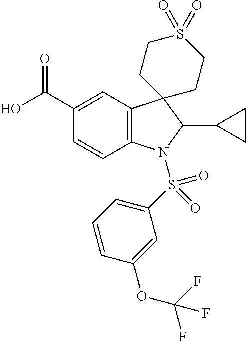 [SMILES: O=C(O)c1ccc2c(c1)C1(CCS(=O)(=O)CC1)C(C1CC1)N2S(=O)(=O)c1cccc(OC(F)(F)F)c1]

F.5 was prepared in analogy to intermediate F.1 according to GP 7 starting from E.5. UPLC-MS (ESI−): [M−H]⁻=544.

### Intermediate F.6

### Preparation of 2-cyclopropyl-1-{[3-(difluoromethoxy)phenyl]sulfonyl}-1,2,2′,3′,5′,6′-hexahydrospiro[indole-3,4′-thiopyran]-5-carboxylic acid 1′,1′-dioxide

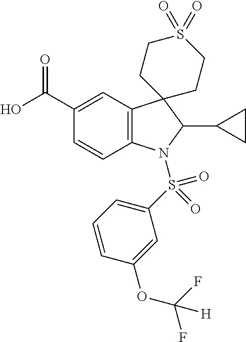 [SMILES: O=C(O)c1ccc2c(c1)C1(CCS(=O)(=O)CC1)C(C1CC1)N2S(=O)(=O)c1cccc(OC(F)F)c1]

F.6 was prepared in analogy to intermediate F.1 according to GP 7 starting from E.6. UPLC-MS (ESI−): [M−H]⁻=526.

### Intermediate F.7

### Preparation of 2-cyclopropyl-1-{[4-(difluoromethoxy)phenyl]sulfonyl}-1,2,2′,3′,5′,6′-hexahydrospiro[indole-3,4′-thiopyran]-5-carboxyic acid 1′,1′-dioxide

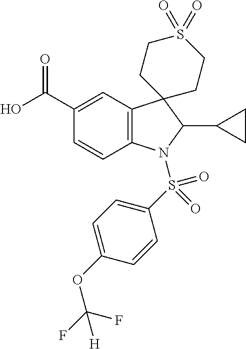 [SMILES: O=C(O)c1ccc2c(c1)C1(CCS(=O)(=O)CC1)C(C1CC1)N2S(=O)(=O)c1ccc(OC(F)F)cc1]

F.7 was prepared in analogy to intermediate F.1 according to GP 7 starting from E.7. UPLC-MS (ESI−): [M−H]⁻=526.

### Intermediate F.8

### Preparation of 1-[(4-cyanophenyl)sulfonyl]-2-cyclopropyl-1,2,2′,3′,5′,6′-hexahydrospiro[indole-3,4′-thiopyran]-5-carboxylic acid 1′,1′-dioxide

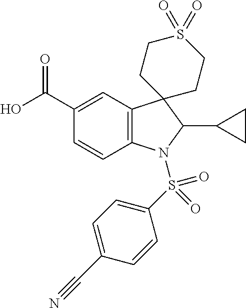 [SMILES: N#Cc1ccc(S(=O)(=O)N2c3ccc(C(=O)O)cc3C3(CCS(=O)(=O)CC3)C2C2CC2)cc1]

F.8 was prepared according to GP 8 starting from D.6. The aryl bromide D.6 (1 g) was placed into a steel autoclave under argon atmosphere and dissolved in dimethyl sulfoxide (30 mL). 25 mg of palladium(II) acetate, 250 mg of 1,1′-bis(diphenylphosphino)ferrocene and 750 mg of potassium acetate were added and the mixture was purged 3 times with carbon monoxide. The mixture was stirred for 30 min at 20° C. under a carbon monoxide pressure of ca. 11.3 bar. The autoclave was set under vacuum again, then a carbon monoxide pressure of ca. 12.69 bar was applied and the mixture heated to 100° C. until TLC and/or LCMS indicate complete consumption of the starting material (24 h), yielding a maximum pressure of ca. 14.9 bar. The reaction was cooled to rt, the pressure released and the reaction mixture given to a mixture of 2 M HCl_{aq} in ice-water. After stirring for 20 min, the formed precipitate was filtered off and washed with water. The obtained crude product was taken to the next step without further purification. UPLC-MS (ESI−): [M−H]⁻=485.

### Intermediate F.9

### Preparation of 1-[(3-cyanophenyl)sulfonyl]-2-cyclopropyl-1,2,2′,3′,5′,6′-hexahydrospiro[indole-3,4′-thiopyran]-5-carboxylic acid 1′,1′-dioxide

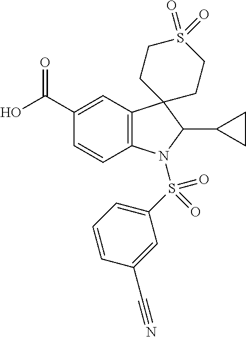 [SMILES: N#Cc1cccc(S(=O)(=O)N2c3ccc(C(=O)O)cc3C3(CCS(=O)(=O)CC3)C2C2CC2)c1]

F.9 was prepared in analogy to intermediate F.8 according to GP 8 starting from D.7. UPLC-MS (ESI−): [M−H]⁻=485.

### Intermediate F.10

### Preparation of 1-[(4-carbamoylphenyl)sulfonyl]-2-cyclopropyl-1,2,2′,3′,5′,6′-hexahydrospiro[indole-3,4′-thiopyran]-5-carboxylic acid 1′,1′-dioxide

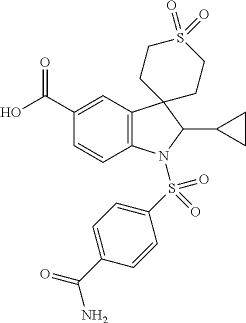 [SMILES: NC(=O)c1ccc(S(=O)(=O)N2c3ccc(C(=O)O)cc3C3(CCS(=O)(=O)CC3)C2C2CC2)cc1]

F.10 was prepared in a modification to GP 8 starting from D.11. Deviating from GP 8 the precipitate obtained upon aqueous work-up was redissolved in ethyl acetate. It was further proceeded as described in GP 8. ¹H-NMR (300 MHz, DMSO-d6): Shift [ppm]=0.16-0.21 (m, 1H), 0.38-0.47 (m, 1H), 0.50-0.65 (m, 2H), 0.83-0.90 (m, 1H), 0.94-1.03 (m, 1H), 1.34-1.44 (m, 1H), 2.50-2.56 (m, 3H), 3.17-3.22 (m, 2H), 3.59-3.69 (m, 1H), 4.38 (d, 1H), 7.60-7.66 (m, 3H), 7.88-7.96 (m, 5H), 8.11 (br. s., 1H), 12.93 (br. s., 1H). UPLC-MS (ESI−): [M−H]⁻=503.

### Intermediate F.11

### Preparation of 1-[(4-fluorophenyl)sulfonyl]-2-(prop-2-en-1-yl)-1,2,2′,3′,5′,6′-hexahydrospiro[indole-3,4′-thiopyran]-5-carboxylic acid 1′,1′-dioxide

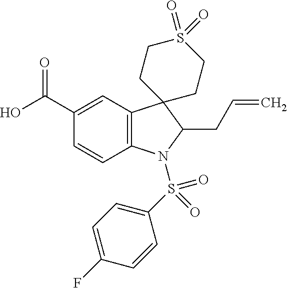 [SMILES: C=CCC1N(S(=O)(=O)c2ccc(F)cc2)c2ccc(C(=O)O)cc2C12CCS(=O)(=O)CC2]

F.11 was prepared in analogy to intermediate F.1 according to GP 7 starting from E.8. ¹H-NMR (300 MHz, CDCl₃): Shift [ppm]=0.80 (m, 1H), 1.77 (tbr, 1H), 2.40 (m, 2H), 2.60-2.88 (m, 3H), 2.90-3.30 (m, 3H), 4.39 (sbr, 1H), 5.00-5.18 (m, 2H), 5.70 (m, 1H), 7.13 (m, 2H), 7.70 (m, 1H), 7.78-8.00 (m, 3H), 8.14 (d, 1H). UPLC-MS (ESI−): [M−H]⁻=478.

### Intermediate F.12

### Preparation of 2-cyclopropyl-1-{[3-(methoxycarbonyl)phenyl]sulfonyl}-1,2,2′,3′,5′,6′-hexahydrospiro[indole-3,4′-thiopyran]-5-carboxyic acid 1′,1′-dioxide

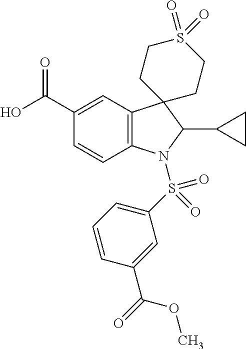 [SMILES: COC(=O)c1cccc(S(=O)(=O)N2c3ccc(C(=O)O)cc3C3(CCS(=O)(=O)CC3)C2C2CC2)c1]

F.12 was prepared in analogy to intermediate F.8 according to GP 8 starting from D.13. UPLC-MS (ESI−): [M−H]⁻=518.

### Intermediate G.1

### Preparation of 2-cyclopropyl-1-[(4-fluorophenyl)sulfonyl]-1,2,2′,3′,5′,6′-hexahydrospiro[indole-3,4′-thiopyran]1′,1′-dioxide

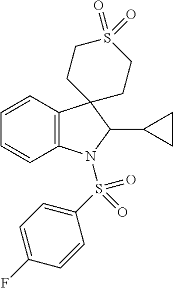 [SMILES: O=S1(=O)CCC2(CC1)c1ccccc1N(S(=O)(=O)c1ccc(F)cc1)C2C1CC1]

According to GP 12 2.90 g (5.63 mmol) of intermediate D.1 were hydrogenated with 1.80 g palladium on charcoal (10% Pd/C; contains 50% of water) for 2.5 h to yield 2.23 g (91%) of the desired debrominated indoline. ¹H-NMR (400 MHz, DMSO-d6): Shift [ppm]=0.25-0.30 (m, 1H), 0.36-0.42 (m, 1H), 0.45-0.52 (m, 1H), 0.55-0.61 (m, 1H), 0.80-0.86 (m, 1H), 0.93-1.01 (m, 1H), 1.41 (dt, 1H), 2.39-2.58 (m, 3H), 3.18-3.21 (m, 2H), 3.58 (dt, 1H), 4.28 (d, 1H), 7.10 (dt, 1H), 7.23 (dd, 1H), 7.30 (dt, 1H), 7.36-7.42 (m, 2H), 7.52 (d, 1H), 7.83-7.88 (m, 2H). UPLC-MS (ESI+): [M+H]⁺=436.

### Intermediate H.1

### Preparation of 2-cyclopropyl-1-[(4-fluorophenyl)sulfonyl]-5-nitro-1,2,2′,3′,5′,6′-hexahydrospiro[indole-3,4′-thiopyran]1′,1′-dioxide

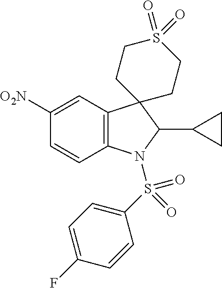 [SMILES: O=[N+]([O-])c1ccc2c(c1)C1(CCS(=O)(=O)CC1)C(C1CC1)N2S(=O)(=O)c1ccc(F)cc1]

According to GP 13 2.23 g (5.11 mmol) of intermediate G.1 were nitrated with 6.4 mL (153 mmol) concentrated nitric acid for 2 h to yield 2.39 g (97%) of the desired nitroarene. ¹H-NMR (300 MHz, DMSO-d6): Shift [ppm]=0.33-0.66 (m, 4H), 0.81-0.89 (m, 1H), 0.96-1.05 (m, 1H), 1.55 (dt, 1H), 2.50-2.56 (m, 1H), 2.61-2.69 (m, 2H), 3.18-3.22 (m, 2H), 3.66 (dt, 1H), 4.45 (d, 1H), 7.41-7.47 (m, 2H), 7.71 (d, 1H), 7.93-7.97 (m, 2H), 8.07 (d, 1H), 8.23 (dd, 1H). UPLC-MS (ESI+): [M+H]⁺=481.

### Intermediate I.1

### Preparation of 2-cyclopropyl-1-[(4-fluorophenyl)sulfonyl]-1,2,2′,3′,5′,6′-hexahydrospiro[indole-3,4′-thiopyran]-5-amine 1′,1′-dioxide

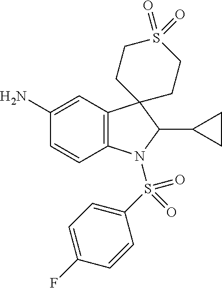 [SMILES: Nc1ccc2c(c1)C1(CCS(=O)(=O)CC1)C(C1CC1)N2S(=O)(=O)c1ccc(F)cc1]

According to GP 14 2.54 g (5.63 mmol) of intermediate H.1 were hydrogenated with 600 mg palladium on charcoal (10% Pd/C) for 4.5 h. In a slight modification to GP 14 the crude product was taken up with ethyl acetate (80 mL). The obtained solid was filtered off, rinsed with a small amount of ethyl acetate (20 mL) and dried to yield a first amount of the desired aniline (1.2 g, 51%). The filtrate was purified by flash chromatography (SiO₂-hexane/ethyl acetate) to give a another amount of product (502 mg, 21%). ¹H-NMR (300 MHz, DMSO-d6): Shift [ppm]=0.11-0.16 (m, 1H), 0.33-0.60 (m, 3H), 0.77-0.85 (m, 1H), 0.91-1.00 (m, 1H), 1.34 (dt, 1H), 2.29-2.50 (m, 3H), 3.15-3.19 (m, 2H), 3.50 (dt, 1H), 4.13 (d, 1H), 5.04 (br. s., 2H), 6.38 (d, 1H), 6.46 (dd, 1H), 7.19 (d, 1H), 7.33-7.39 (m, 2H), 7.76-7.80 (m, 2H). UPLC-MS (ESI+): [M+H]⁺=451.

Compounds according to the invention:

### Example 1

### N-[(3-Chloropyridin-2-yl)methyl]-1-[(4-fluorophenyl)sulfonyl]-2-methyl-1,2,2′,3′,5′,6′-hexahydrospiro[indole-3,4′-pyran]-5-carboxamide

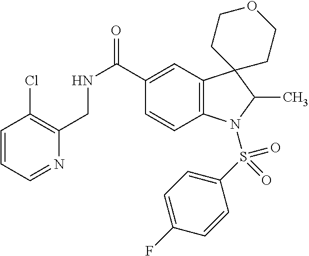 [SMILES: CC1N(S(=O)(=O)c2ccc(F)cc2)c2ccc(C(=O)NCc3ncccc3Cl)cc2C12CCOCC2]

According to GP 9.1, 250 mg (0.62 mmol) of intermediate F.3 and 105 mg (0.74 mmol, 1.2 eq.) 1-(3-chlorpyridin-2-yl)methylamine (CAS No. [500305-98-6]) were reacted with 280 mg (0.74 mmol, 1.2 eq.) HATU in the presence of 0.32 mL (0.23 mmol, 3.7 eq.) triethylamine in 10 mL DMF to yield 300 mg (84%) of the desired amide. ¹H-NMR (300 MHz, DMSO-d6): Shift [ppm]=−0.02 (d, 1H), 0.97-1.08 (m, 1H), 1.25 (d, 3H), 1.67 (d, 1H), 1.96-2.08 (m, 1H), 3.32-3.50 (m, 3H), 3.79-3.87 (m, 1H), 4.50 (q, 1H), 4.63 (d, 2H), 7.33 (dd, 1H), 7.35-7.41 (m, 2H), 7.54 (d, 1H), 7.76 (d, 1H), 7.81 (dd, 1H), 7.87-7.92 (m, 3H), 8.44 (dd, 1H), 8.80 (t, 1H). UPLC-MS (ESI+): [M+H]⁺=530/532 (CI isotope pattern).

The enantiomers of the racemic material of example 1 were separated by chiral preparative HPLC (System: Dionex: Pump P 580, Gilson: Liquid Handler 215, Knauer: UV-Detektor K-2501; Column: Chiralpak IC 5 μm 250×30 mm; Solvent: Hexane/ethanol 70:30; Flow: 40 mL/min; Temperature: rt; Injection: 1.0 mL/run, 68 mg/mL THF; Detection: UV 280 nm) and analytically characterized by HPLC method A with Column: Chiralpak IC 5 μm 150×4.6 mm; Solvent: hexane/ethanol 70:30; Detection: DAD 280 nm:

### Example 1.1

Rt=17.81 min;

### Example 1.2

Rt=23.01 min;

TABLE 1
The following examples (3 to 11) were prepared in analogy to example 1 starting
from intermediate F.3 and commercially available amines, applying the indicated general
procedure. Example 2 was prepared from intermediate C.5 according to the given procedure.

| No | Structure | Name | Analytical data | Methods |
|---|---|---|---|---|
| 2 | 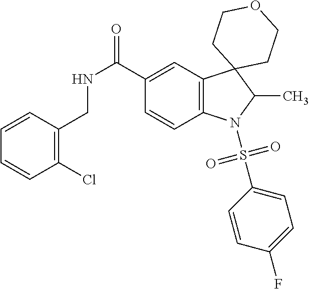 [SMILES: CC1N(S(=O)(=O)c2ccc(F)cc2)c2ccc(C(=O)NCc3ccccc3Cl)cc2C12CCOCC2] | N-(2-chlorobenzyl)- 1-[(4- fluorophenyl) sulfonyl]-2-methyl- 1,2,2′,3′,5′,6′- hexahydrospiro [indole-3,4′-pyran]-5- carboxamide | ¹H-NMR (300 MHz, DMSO-d6): Shift [ppm] = 0.02 (d, 1H), 1.01- 2.08 (m, 1H), 1.26 (d, 3H), 1.65- 1.69 (m, 1H), 1.98-2.06 (m, 1H), 3.33-3.41 (m, 2H), 3.43-3.50 (m, 1H), 3.81-3.86 (m, 1H), 4.49 (d, 2H), 4.52 (t, 1H), 7.24-7.30 (m, 2H), 7.30-7.34 (m, 1H), 7.35- 7.43 (m, 3H), 7.54 (d, 1H), 7.76 (d, 1H), 7.83 (dd, 1H), 7.88-7.92 (m, 2H), 8.88 (t, 1H). UPLC-MS (ESI+): [M + H]⁺ = 529/531 (CI isotope pattern). | prepared by carbonylation of intermediate C.5 according to GP 10 |
| 2.1 | Enantiomer 1 of Ex. 2 | | Rₜ = 17.6 min | HPLC method |
| 2.2 | Enantiomer 1 of Ex. 2 | | Rₜ = 18.8 min | A with Column: |
| | | | | Chiralpak IC |
| | | | | 5 μm 150 × 4.6 |
| | | | | mm; Solvent: |
| | | | | hexane/ |
| | | | | ethanol 85:15; |
| | | | | Detection: |
| | | | | DAD 280 nm |
| 3 | 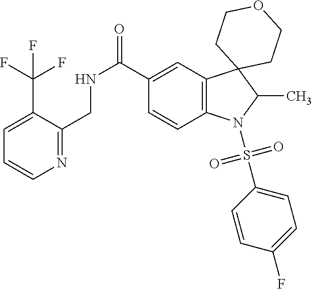 [SMILES: CC1N(S(=O)(=O)c2ccc(F)cc2)c2ccc(C(=O)NCc3ncccc3C(F)(F)F)cc2C12CCOCC2] | 1-[(4- fluorophenyl) sulfonyl]-2-methyl- N-{[3-(trifluoromethyl) pyridin-2-yl]methyl}- 1,2,2′,3′,5′,6′- hexahydrospiro [indole-3,4′-pyran]-5- carboxamide | ¹H-NMR (300 MHz, DMSO-d6): Shift [ppm] = −0.02 (d, 1H), 0.96- 1.10 (m, 1H), 1.25 (d, 3H), 1.67 (d, 1H), 1.94-2.08 (m, 1H), 3.32- 3.50 (m, 3H), 3.77-3.86 (m, 1H), 4.51 (q, 1H), 4.69 (d, 2H), 7.35- 7.41 (m, 2H), 7.50 (dd, 1H), 7.55 (d, 1H), 7.76 (d, 1H), 7.81 (dd, 1H), 7.88-7.92 (m, 2H), 8.14 (d, 1H), 8.75 (d, 1H), 8.89 (t, 1H). UPLC-MS (ESI+): [M + H]⁺ = 564 | GP 9.1 |
| 3.1 | Enantiomer 1 of Ex. 3 | | Rₜ = 7.3 min | HPLC method |
| 3.2 | Enantiomer 2 of Ex. 3 | | Rₜ = 8.2 min | A with Column: |
| | | | | Chiralpak IC |
| | | | | 5 μm 150 × 4.6 |
| | | | | mm; Solvent: |
| | | | | hexane/ |
| | | | | ethanol 70:30 |
| | | | | (v/v); |
| | | | | Detection: |
| | | | | DAD 254 nm. |
| 4 | 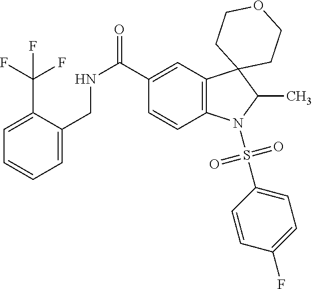 [SMILES: CC1N(S(=O)(=O)c2ccc(F)cc2)c2ccc(C(=O)NCc3ccccc3C(F)(F)F)cc2C12CCOCC2] | 1-[(4- fluorophenyl) sulfonyl]-2-methyl-N- [2-(trifluoromethyl) benzyl]-1,2,2′,3′,5′,6′- hexahydrospiro [indole-3,4′-pyran]-5- carboxamide | ¹H-NMR (400 MHz, DMSO-d6): Shift [ppm] = 0.02 (d, 1H), 1.05 (dt, 1H), 1.26 (d, 3H), 1.68 (d, 1H), 2.03 (dt, 1H), 3.34-3.40 (m, 2H), 3.43-3.50 (m, 1H), 3.81-3.86 (m, 1H), 4.52 (q, 1H), 4.61 (d, 2H), 7.36-7.49 (m, 4H), 7.56 (d, 1H), 7.61 (t, 1H), 7.70 (d, 1H), 7.77 (d, 1H), 7.84 (dd, 1H), 7.89-7.93 (m, 2H), 8.95 (t, 1H). UPLC-MS (ESI+): [M + H]⁺ = 563 | GP 9.1 |
| 5 | 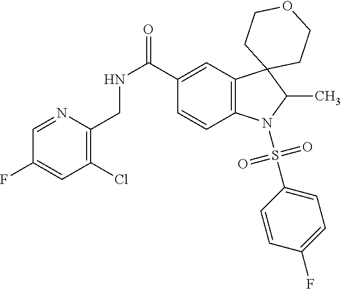 [SMILES: CC1N(S(=O)(=O)c2ccc(F)cc2)c2ccc(C(=O)NCc3ncc(F)cc3Cl)cc2C12CCOCC2] | N-[(3-chloro-5- fluoropyridin-2- yl)methyl]-1-[(4- fluorophenyl) sulfonyl]-2-methyl- 1,2,2′,3′,5′,6′- hexahydrospiro [indole-3,4′-pyran]-5- carboxamide | ¹H-NMR (300 MHz, DMSO-d6): Shift [ppm] = −0.03 (d, 1H), 0.94- 1.08 (m, 1H), 1.24 (d, 3H), 1.65 (d, 1H), 1.94-2.06 (m, 1H), 3.26- 3.50 (m, 3H), 3.78-3.85 (m, 1H), 4.49 (q, 1H), 4.55 (d, 2H), 7.34- 7.40 (m, 2H), 7.53 (d, 1H), 7.72 (s, 1H), 7.79 (dd, 1H), 7.86-7.91 (m, 2H), 8.03 (dd, 1H), 8.42 (s, 1H), 8.90 (t, 1H). UPLC-MS (ESI+): [M + H]⁺ = 548/550 (CI isotope pattern). | GP 9.1 |
| 6 | 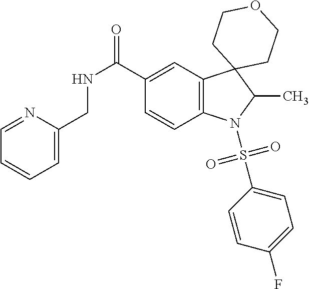 [SMILES: CC1N(S(=O)(=O)c2ccc(F)cc2)c2ccc(C(=O)NCc3ccccn3)cc2C12CCOCC2] | 1-[(4- fluorophenyl sulfonyl]-2-methyl-N- (2-pyridylmethyl)- 1,2,2′,3′,5′,6′- hexahydrospiro [indole-3,4′-pyran]-5- carboxamide | ¹H-NMR (400 MHz, DMSO-d6): Shift [ppm] = 0.00 (d, 1H), 1.04 (dt, 1H), 1.25 (d, 3H), 1.67 (d, 1H), 2.03 (dt, 1H), 3.33-3.41 (m, 2H), 3.46 (m, 1H), 3.84 (m, 1H), 4.48- 4.54 (m, 3H), 7.22 (dd, 1H), 7.28 (d, 1H), 7.36-7.40 (m, 2H), 7.54 (d, 1H), 7.71 (dt, 1H), 7.77 (d, 1H), 7.83 (dd, 1H), 7.88-7.91 (m, 2H), 8.47 (d, 1H), 8.97 (t, 1H). UPLC-MS (ESI+): [M + H]⁺ = 496 | GP 9.1 |
| 7 | 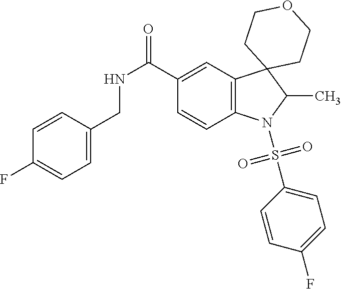 [SMILES: CC1N(S(=O)(=O)c2ccc(F)cc2)c2ccc(C(=O)NCc3ccc(F)cc3)cc2C12CCOCC2] | N-(4-fluorobenzyl)- 1-[(4- fluorophenyl) sulfonyl]-2-methyl- 1,2,2′,3′,5′,6′- hexahydrospiro [indole-3,4′-pyran]-5- carboxamide | ¹H-NMR (300 MHz, DMSO-d6): Shift [ppm] = −0.05 (d, 1H), 1.02 (dt, 1H), 1.24 (d, 3H) 1.65 (d, 1H), 2.01 (dt, 1H), 3.31-3.40 (m, 2H), 3.42-3.50 (m, 1H), 3.79-3.86 (m, 1H), 4.39 (d, 2H), 4.50 (q, 1H), 7.08-7.14 (m, 2H), 7.28-7.33 (m, 2H), 7.34-7.40 (m, 2H), 7.53 (d, 1H), 7.72 (d, 1H), 7.80 (dd, 1H), 7.86-7.91 (m, 2H), 8 90 (t, 1H). UPLC-MS (ESI+): [M + H]⁺ = 513 | GP 9.1 |
| 8 | 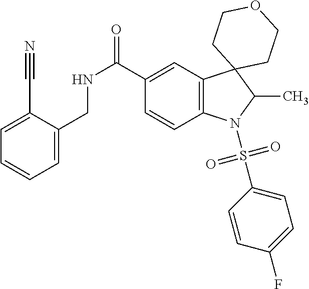 [SMILES: CC1N(S(=O)(=O)c2ccc(F)cc2)c2ccc(C(=O)NCc3ccccc3C#N)cc2C12CCOCC2] | N-(2-cyanobenzyl)- 1-[(4- fluorophenyl) sulfonyl]-2-methyl- 1,2,2′,3′,5′,6′- hexahydrospiro [indole-3,4′-pyran]-5- carboxamide | ¹H-NMR (400 MHz, DMSO-d6): Shift [ppm] = 0.02 (d, 1H), 1.03 (dt, 1H), 1.25 (d, 3H), 1.66 (d, 1H), 1.92-2.06 (m, 1H), 3.34-3.41 (m, 2H), 3.43-3.49 (m, 1H), 3.81- 3.85 (m, 1H), 4.51 (q, 1H), 4.59 (d, 2H), 7.36-7.49 (m, 4H), 7.54 (d, 1H ), 7.57-7.66 (m, 1H), 7.74 (d, 1H), 7.74-7.83 (m, 1H), 7.87- 7.92 (m, 3H), 9.03 (t, 1H). UPLC-MS (ESI+): [M + H]⁺ = 520 | GP 9.1 |
| 9 | 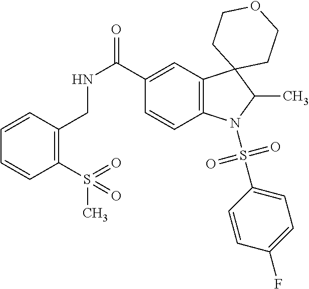 [SMILES: CC1N(S(=O)(=O)c2ccc(F)cc2)c2ccc(C(=O)NCc3ccccc3S(C)(=O)=O)cc2C12CCOCC2] | 1-[(4- fluorophenyl) sulfonyl]-N-(2- mesylbenzyl)-2- methyl- 1,2,2′,3′,5′,6′- hexahydrospiro [indole-3,4′-pyran]-5- carboxamide | ¹H-NMR (400 MHz, DMSO-d6): Shift [ppm] = 0.02 (d, 1H), 1.05 (dt, 1H), 1.26 (d, 3H), 1.68 (d, 1H), 2.04 (dt, 1H), 3.34-3.41 (m, 2H), 3.35 (s, 3H), 3.44-3.51 (m, 1H), 3.81-3.86 (m, 1H), 4.52 (q, 1H), 4.82 (d, 2H), 7.36-7.41 (m, 2H), 7.49 (t, 1H), 7.55 (d, 1H), 7.66 (dt, 1H), 7.77 (d, 1H), 7.83 (dd, 1H), 7.89-7.92 (m, 3H), 9.10 (t, 1H). UPLC-MS (ESI+): [M + H]⁺ = 573 | GP 9.1 |
| 10 | 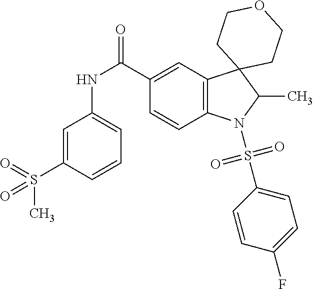 [SMILES: CC1N(S(=O)(=O)c2ccc(F)cc2)c2ccc(C(=O)Nc3cccc(S(C)(=O)=O)c3)cc2C12CCOCC2] | 1-[(4- fluorophenyl) sulfonyl]-N-(3- mesylphenyl)-2- methyl- 1,2,2′,3′,5′,6′- hexahydrospiro [indole-3,4′-pyran]-5- carboxamide | ¹H-NMR (400 MHz, DMSO-d6): Shift [ppm] = 0.26 (d, 1H), 1.14 (dt, 1H), 1.30 (d, 3H), 1.73 (d, 1H), 2.12 (dt, 1H), 3.20 (s, 3H), 3.37- 3.44 (m, 2H), 3.52 (dt, 1H), 3.89 (d, 1H), 4.58 (q, 1H), 7.40-7.45 (m, 2H), 7.61-7.64 (m, 3H), 7.86 (d, 1H), 7.92-7.97 (m, 3H), 8.09- 8.11 (m, 1H), 8.34 (s, 1H), 10.41 (s, 1H). UPLC-MS (ESI+): [M + H]⁺ = 559 | GP 9.1 |
| 11 | 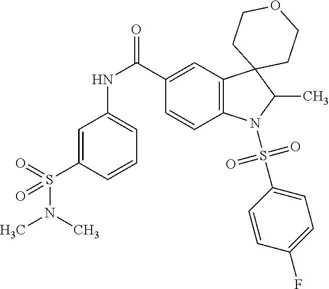 [SMILES: CC1N(S(=O)(=O)c2ccc(F)cc2)c2ccc(C(=O)Nc3cccc(S(=O)(=O)N(C)C)c3)cc2C12CCOCC2] | N-[3-(N,N- dimethylsulfamoyl) phenyl]-1-[(4- fluorophenyl) sulfonyl]-2-methyl- 1,2,2′,3′,5′,6′- hexahydrospiro [indole-3,4′-pyran]-5- carboxamide | ¹H-NMR (400 MHz, DMSO-d6): Shift [ppm] = 0.03 (d, 1H), 1.11 (dt, 1H), 1.27 (d, 3H), 1.69 (d, 1H), 2.10 (dt, 1H), 2.60 (s, 6H), 3.35- 3.43 (m, 2H), 3.49 (dt, 1H), 3.86 (d, 1H), 4.55 (q, 1H), 7.36-7.44 (m, 3H), 7.58-7.62 (m, 2H), 7.82 (d, 1H), 7.89-7.95 (m, 3H), 8.04- 8.09 (m, 1H), 8.15 (t, 1H), 10.37 (s, 1H). UPLC-MS (ESI+): [M + H]⁺ = 588 | GP 9.1 |

### Example 12

### N-(2-Chlorobenzyl)-2-cyclopropyl-1-[(4-fluorophenyl)sulfonyl]-1,2,2′,3′,5′,6′-hexahydrospiro[indole-3,4′-thiopyran]-5-carboxamide

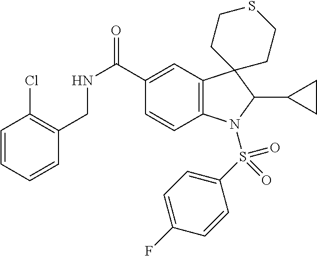 [SMILES: O=C(NCc1ccccc1Cl)c1ccc2c(c1)C1(CCSCC1)C(C1CC1)N2S(=O)(=O)c1ccc(F)cc1]

In an adaption of GP 10: 2.70 g (5.60 mmol) of intermediate C.1 were dissolved in 40 mL 1,4-dioxane (with 0.1 mL water) and 2.38 g (3 eq.) 2-chlorobenzylamine (CAS No. [89-97-4]), 1.48 g (1 eq.) molybdenum hexacarbonyl, 1.78 g (3 eq.) sodium carbonate, 162 mg (0.1 eq.) tri-tert-butylphosphonium tetrafluoroborate and 126 mg (0.1 eq.) palladium(II) acetate were added. The mixture was heated to reflux (bath temperature 120° C.) for 18 h. After cooling to rt, the solids were filtered off and rinsed with ethyl acetate. The combined filtrates were washed with water, dried with sodium sulfate and the solvents removed in vacuo. The crude product was purified by flash chromatography (yield: 31%) or preparative HPLC, respectively. ¹H-NMR (300 MHz, DMSO-d6): Shift [ppm]=0.25 (d, 1H), 0.32-0.49 (m, 2H), 0.52-0.63 (m, 1H), 0.67-0.77 (m, 1H), 0.87-1.07 (m, 2H), 1.98 (d, 1H), 2.07-2.22 (m, 1H), 2.35 (d, 1H), 2.59-2.69 (m, 1H), 2.82 (m, 2H), 4.03 (d, 1H), 4.48 (d, 2H), 7.23-7.43 (m, 6H), 7.53 (d, 1H), 7.76 (s, 1H), 7.81-7.88 (m, 3H), 8.94 (t, 1H); UPLC-MS (ESI+): [M+H]⁺=571/573 (CI isotope pattern).

The enantiomers of the racemic material of example 12 were separated by chiral preparative HPLC (System: Dionex: Pump P 580, Gilson: Liquid Handler 215, Knauer: UV-Detektor K-2501; Column: Chiralpak IC 5 μm 250×30 mm; Solvent: hexane/ethanol 90:10; Flow: 40 mL/min; Temperature: rt; Injection: 0.75 mL/run, 63 mg/mL THF; Detection: UV 280 nm) and analytically characterized by HPLC method A with Column: Chiralpak IC 5 μm 150×4.6 mm; Solvent: hexane/ethanol 90:10; Detection: DAD 280 nm:

### Example 12.1

Rt=18.04 min (enantiomer 1)

### Example 12.2

Rt=20.35 min (enantiomer 2)

### Example 13

### N-(2-Chlorobenzyl)-2-cyclopropyl-1-[(4-fluorophenyl)sulfonyl]-1,2,2′,3′,5′,6′-hexahydrospiro[indole-3,4′-thiopyran]-5-carboxamide 1′-oxide

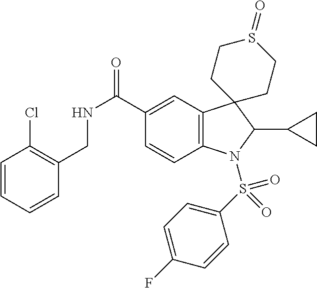 [SMILES: O=C(NCc1ccccc1Cl)c1ccc2c(c1)C1(CCS(=O)CC1)C(C1CC1)N2S(=O)(=O)c1ccc(F)cc1]

According to GP 11 250 mg (0.44 mmol) of example compound 12 were dissolved at rt in 12 mL acetonitrile, 10 mg (0.06 mmol, 0.14 eq.) iron(III) chloride were added and after min stirring, 110 mg (0.48 mmol, 1.1 eq.) periodic acid were added. After 45 min stirring at rt, the mixture was partitioned between ethyl acetate and half-saturated aqueous sodium hydrocarbonate. The layers were separated and the aqueous phase (pH ˜10) extracted with ethyl acetate. The combined organic layers were washed with water and brine, dried with sodium sulfate and the solvents removed in vacuo. The crude product (yield: 78%) was purified by preparative HPLC. The product was obtained as a 3:1 mixture of sulfoxide-diastereomers. ¹H-NMR (300 MHz, DMSO-d6, major isomer:): Shift [ppm]=−0.19 (d, 1H), 0.33-0.44 (m, 2H), 0.48-0.63 (m, 1H), 0.71-0.84 (m, 1H), 0.88-1.00 (m, 1H), 1.64 (m, 1H), 1.97-2.35 (m, 2H), 2.62-2.82 (m, 2H), 2.95 (m, 2H), 4.16 (d, 1H) [minor isomer: 4.11 (d, 1H)], 4.48 (d, 2H), 7.22-7.43 (m, 6H), 7.54 (d, 1H) [minor isomer: 7.56 (d, 1H)], 7.75-7.92 (m, 4H), 9.05 (t, 1H) [minor isomer: 8.91 (t, 1H)]. UPLC-MS (ESI+): [M+H]⁺=587/589 (CI isotope pattern).

### Example 13.1 and Example 13.2

The enantiopure sulfides 12.1 and 12.2 were oxidized to the corresponding sulfoxides 13.1 and 13.2 according to the same procedure as given for the racemate 12. The crude products were purified by preparative HPLC to obtain the major sulfoxide isomer, respectively.

### Example 14

### N-[(3-Chloropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide

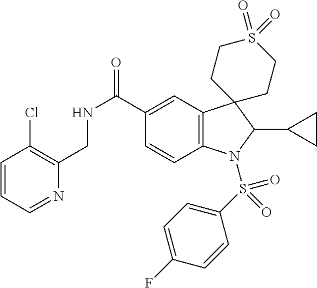 [SMILES: O=C(NCc1ncccc1Cl)c1ccc2c(c1)C1(CCS(=O)(=O)CC1)C(C1CC1)N2S(=O)(=O)c1ccc(F)cc1]

According to GP 9.1 4.34 mmol of intermediate F.1 and 8.67 mmol 1-(3-chlorpyridin-2-yl)methylamine (CAS No. [500305-98-6]) were reacted with 6.51 mmol HATU in the presence of 1.81 mL (13 mmol) triethylamine in 170 mL DMF to yield 2.40 g (85%) of the desired amide. ¹H-NMR (300 MHz, DMSO-d6): Shift [ppm]=0.22 (d, 1H), 0.33-0.61 (m, 3H), 0.77-0.85 (m, 1H), 0.91-1.02 (m, 1H), 1.35-1.49 (m, 1H), 2.40-2.63 (m, 3H), 3.10-3.23 (m, 2H), 3.54-3.67 (m, 1H), 4.33 (d, 1H), 4.63 (d, 2H), 7.30-7.40 (m, 3H), 7.56 (d, 1H), 7.81-7.90 (m, 5H), 8.44 (d, 1H), 8.96 (t, 1H); UPLC-MS (ESI+): [M+H]⁺=604/606 (CI isotope pattern).

The enantiomers of the racemic material of example 14 were separated by chiral preparative HPLC (System: Dionex: Pump P 580, Gilson: Liquid Handler 215, Knauer: UV-Detektor K-2501; Column: Chiralpak IA 5 μm 250×30 mm; Solvent: Methanol/0.1% diethylamine; Flow: mL/min; Temperature: rt; Injection: 0.6 mL/run, 130 mg/mL DMSO/methanol; Detection: UV 280 nm) and analytically characterized by HPLC (method B1 with Column: Chiralpak IC 5 μm 150×6.6 mm; Solvent: Methanol/0.1% diethylamine) and specific optical rotation:

### Example 14.1

Rₜ=5.12 min; [α]_{D}²⁰=−109.5°+/−0.21° (C=0.60, chloroform)

### Example 14.2

Rₜ=6.65 min; [α]_{D}²⁰=+108.5°+/−0.13° (C=0.61, chloroform)

TABLE 2
The following examples were prepared in analogy to example 14 starting from the
corresponding acid intermediates F.1, F.2, F.4, F.5, F.6, F.7, F.8, F.9, F.10, F.11 or F.12 and
commercially available amines, applying the indicated general procedure. Examples 49.1,
49.2, 50, 51.1, 51.2, 54, 55, 56, 59, 61.1, 61.2, 81, 83, 86, 90.1, 90.2 and 94 were prepared
according to the given procedures.

| No | Structure | Name | Analytical data | Methods |
|---|---|---|---|---|
| 15 | 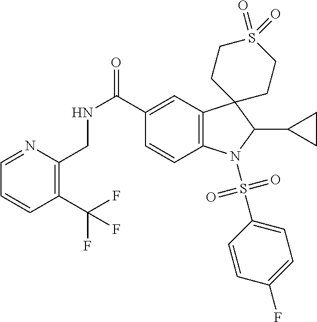 [SMILES: O=C(NCc1ncccc1C(F)(F)F)c1ccc2c(c1)C1(CCS(=O)(=O)CC1)C(C1CC1)N2S(=O)(=O)c1ccc(F)cc1] | 2-cyclopropyl-1-[(4- fluorophenyl) sulfonyl]-N-{[3- (trifluoromethyl)pyridin- 2-yl]methyl}- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.28 (d, 1H), 0.37- 0.44 (m, 1H), 0.47-0.53 (m, 1H), 0.56-0.63 (m, 1H), 0.81- 0.87 (m, 1H), 0.95-1.04 (m, 1H), 1.46 (dt, 1H), 2.53-2.66 (m, 3H), 3.18-3.25 (m, 2H), 3.62 (dt, 1H), 4.35 (d, 1H), 4.67-4.77 (m, 2H), 7.40 (t, 2H), 7.53 (dd, 1H), 7.58 (d, 1H), 7.84-7.91 (m, 4H), 8.16 (d, 1H), 8.78 (d, 1H), 9.03 (t, 1H). UPLC-MS (ESI+): [M + H]⁺ = 638. | from F.1 according to GP 9.1 |
| 15.1 | Enantiomer 1 of Ex. 15 | | Rₜ = 6.8 min | HPLC method |
| 15.2 | Enantiomer 2 of Ex. 15 | | Rₜ = 8.2 min | A with |
| | | | | Column: |
| | | | | Chiralpak IB |
| | | | | 5 μm |
| | | | | 150 × 4.6 |
| | | | | mm; Solvent: |
| | | | | hexane/ |
| | | | | ethanol 70:30 |
| | | | | (v/v); |
| | | | | Detection: |
| | | | | DAD 254 nm |
| 16 | 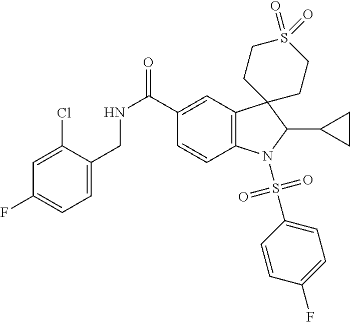 [SMILES: O=C(NCc1ccc(F)cc1Cl)c1ccc2c(c1)C1(CCS(=O)(=O)CC1)C(C1CC1)N2S(=O)(=O)c1ccc(F)cc1] | N-(2-chloro-4- fluorobenzyl)-2- cyclopropyl-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.29 (d, 1H), 0.37- 0.44 (m, 1H), 0.46-0.53 (m, 1H), 0.56-0.62 (m, 1H), 0.81- 0.87 (m, 1H), 0.95-1.03 (m, 1H), 1.47 (dt, 1H), 2.53-2.66 (m, 3H), 3.18-3.24 (m, 2H), 3.62 (dt, 1H), 4.36 (d, 1H), 4.49 (d, 2H), 7.20 (dt, 1H), 7.37-7.42 (m, 3H), 7.44 (dd, 1H), 7.58 (d, 1H), 7.82 (d, 1H), 7.87-7.91 (m, 3H), 9.01 (t, 1H). UPLC-MS (ESI+): [M + H]⁺ = 621/623 (Cl isotope pattern). | from F.1 according to GP 9.1 |
| 16.1 | Enantiomer 1 of Ex. 16 | | Rₜ = 5.1 min | HPLC method |
| 16.2 | Enantiomer 2 of Ex. 16 | | Rₜ = 7.0 min | B1 with |
| | | | | Column: |
| | | | | Chiralpak IA |
| | | | | 5 μm |
| | | | | 150 × 4.6 |
| | | | | mm; Solvent: |
| | | | | Hexane/ |
| | | | | ethanol 60:40 |
| | | | | +0.1% |
| | | | | diethylamine |
| 17 | 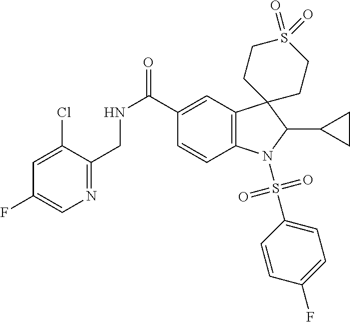 [SMILES: O=C(NCc1ncc(F)cc1Cl)c1ccc2c(c1)C1(CCS(=O)(=O)CC1)C(C1CC1)N2S(=O)(=O)c1ccc(F)cc1] | N-[(3-chloro-5- fluoropyridin-2- yl)methyl]-2- cyclopropyl-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.24 (d, 1H), 0.34- 0.40 (m, 1H), 0.42-0.49 (m, 1H), 0.53-0.60 (m, 1H), 0.77- 0.84 (m, 1H), 0.92-1.00 (m, 1H), 1.43 (dt, 1H), 2.51-2.61 (m, 2H), 3.14-3.22 (m, 3H), 3.59 (dt, 1H), 4.31 (d, 1H), 4.56 (d, 2H), 7.33-7.38 (m, 2H), 7.54 (d, 1H), 7.77 (d, 1H), 7.81-7.87 (m, 3H), 8.01 (dd. 1H), 8.42 (d, 1H), 9.02 (t, 1H) UPLC-MS (ESI+): [M + H]⁺ = 622/624 (Cl isotope pattern). | from F.1 according to GP 9.1 |
| 17.1 | Enantiomer 1 of Ex. 17 | | Rₜ = 4.46 min | HPLC method |
| 17.2 | Enantiomer 2 of Ex. 17 | | Rₜ = 5.83 min | B1 with |
| | | | | Column: |
| | | | | Chiralpak IC |
| | | | | 5 μm |
| | | | | 150 × 4.6 |
| | | | | mm; Solvent: |
| | | | | Methanol/ |
| | | | | 0.1% |
| | | | | diethylamine |
| 18 | 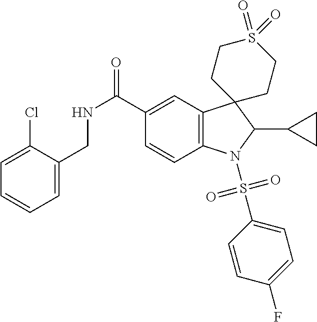 [SMILES: O=C(NCc1ccccc1Cl)c1ccc2c(c1)C1(CCS(=O)(=O)CC1)C(C1CC1)N2S(=O)(=O)c1ccc(F)cc1] | N-(2-chlorobenzyl)- 2-cyclopropyl-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.24 (d, 1H), 0.32- 0.60 (m, 3H), 0.77-0.86 (m, 1H), 0.91-1.02 (m, 1H), 1.45 (m, 1H), 2.40-2.65 (m, 3H), 3.12-3.24 (m, 2H), 3.60 (m, 1H), 4.33 (d, 1H), 4.48 (d, 2H), 7.22-7.43 (m, 6H), 7.56 (d, 1H), 7.81-7.88 (m, 4H), 9.01 (t, 1H). UPLC-MS (ESI+): [M + H]⁺ = 603/605 (Cl isotope pattern). | from F.1 according to GP 9.1 |
| 18.1 | Enantiomer 1 of Ex. 18 | | Rₜ = 4.85 min | HPLC method |
| 18.2 | Enantiomer 2 of Ex. 18 | | Rₜ = 6.89 min | B1 with |
| | | | | Column: |
| | | | | Chiralpak IA |
| | | | | 5 μm |
| | | | | 150 × 4.6 |
| | | | | mm; Solvent: |
| | | | | Hexane/ |
| | | | | ethanol 60:40 |
| | | | | +0.1% |
| | | | | diethylamine |
| 19 | 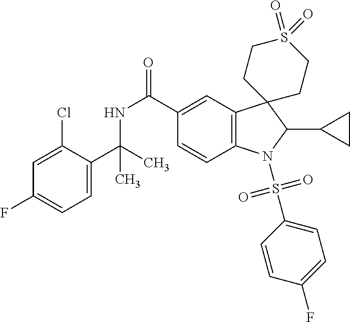 [SMILES: CC(C)(NC(=O)c1ccc2c(c1)C1(CCS(=O)(=O)CC1)C(C1CC1)N2S(=O)(=O)c1ccc(F)cc1)c1ccc(F)cc1Cl] | N-(2-chloro-4- fluoro-α,α- dimethylbenzyl)-2- cyclopropyl-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.22 (d, 1H), 0.32- 0.41 (m, 1H), 0.41-0.49 (m, 1H), 0.50-0.60 (m, 1H), 0.76- 0.85 (m, 1H), 0.89-0.99 (m, 1H), 1.43 (dt, 1H), 1.72 (s, 6H), 2.43- 2.62 (m, 3H), 3.14-3.21 (m, 2H), 3.59 (dt, 1H), 4.32 (d, 1H), 7.14 (dt, 1H), 7.21 (dd, 1H), 7.33- 7.39 (m, 2H), 7.49 (d, 1H), 7.53 (dd, 1H), 7.69 (d, 1H), 7.74 (dd, 1H), 7.82-7.87 (m, 2H), 8.53 (s, 1H). UPLC-MS (ESI+): [M + H]⁺ = 649/651 (Cl isotope pattern). | from F.1 according to GP 9.1 |
| 20 | 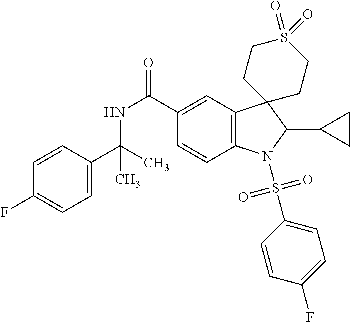 [SMILES: CC(C)(NC(=O)c1ccc2c(c1)C1(CCS(=O)(=O)CC1)C(C1CC1)N2S(=O)(=O)c1ccc(F)cc1)c1ccc(F)cc1] | 2-cyclopropyl-N-(4- fluoro-α,α- dimethylbenzyl)-1- [(4-fluorophenyl) sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.25 (d, 1H), 0.36- 0.43 (m, 1H), 0.45-0.51 (m, 1H), 0.56-0.62 (m, 1H), 0.81- 0.88 (m, 1H), 0.94-1.02 (m, 1H), 1.50 (dt, 1H), 1.64 (s, 6H), 2.43- 2.53 (m, 1H), 2.56-2.67 (m, 2H), 3.16-3.26 (m, 2H), 3.64 (dt, 1H), 4.37 (d, 1H), 7.06 (t, 1H), 7.35-7.42 (m, 4H), 7.53 (d, 1H), 7.74 (d, 1H), 7.79 (dd, 1H), 7.89 (dd, 2H), 7.90 (d, 1H), 8.40 (s, 1H). UPLC-MS (ESI+): [M + H]⁺ = 615. | from F.1 according to GP 9.1 |
| 21 | 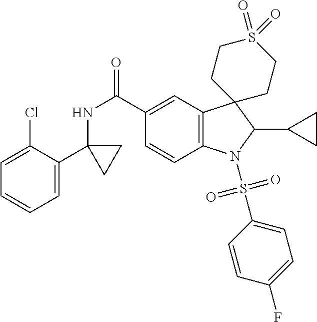 [SMILES: O=C(NC1(c2ccccc2Cl)CC1)c1ccc2c(c1)C1(CCS(=O)(=O)CC1)C(C1CC1)N2S(=O)(=O)c1ccc(F)cc1] | N-[1-(2- chlorophenyl) cyclopropyl]-2- cyclopropyl-1-[(4- fluorophenyl)sulfonyl]- 1,2,2′,3′,5′,6′- hexahydrospiro[indole- 3,4′-thiopyran]- 5-carboxamide 1′,1′-dioxide | 1H-NMR (400 MHz, DMSO-d6): Shift [ppm] = 0.18 (d, 1H), 0.34- 0.40 (m, 1H), 0.40-0.47 (m, 1H), 0.53-0.60 (m, 1H), 0.77- 0.86 (m, 1H), 0.91-0.99 (m, 1H), 1.08-1.17 (m, 2H), 1.19-1.27 (m, 2H), 1.43 (dt, 1H), 2.39- 2.46 (m, 1H), 2.53-2.66 (m, 2H), 3.15-3.25 (m, 2H), 3.60 (dt, 1H), 4.33 (d, 1H), 7.21-7.28 (m, 2H), 7.33-7.39 (m, 3H), 7.51 (d, 1H), 7.71-7.75 (m, 2H), 7.77 (dd, 1H), 7.82-7.86 (m, 2H), 9.02 (s, 1H). UPLC-MS (ESI+): [M + H]⁺ = 629/631 (Cl isotope pattern). | from F.1 according to GP 9.1 |
| 22 | 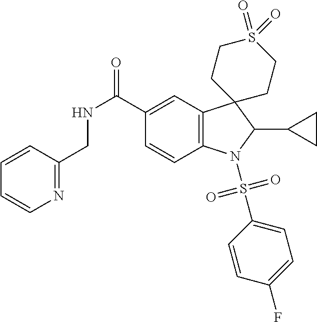 [SMILES: O=C(NCc1ccccn1)c1ccc2c(c1)C1(CCS(=O)(=O)CC1)C(C1CC1)N2S(=O)(=O)c1ccc(F)cc1] | 2-cyclopropyl-1-[(4- fluorophenyl) sulfonyl]-N-(2- pyridylmethyl)- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.23 (d, 1H), 0.32- 0.62 (m, 3H), 0.75-0.87 (m, 1H), 0.89-1.03 (m, 1H), 1.40- 1.49 (m, 1H), 2.40-2.64 (m, 3H), 3.11-3.25 (m, 2H), 3.60 (m, 1H), 4.33 (d, 1H), 4.51 (d, 2H), 7.20-7.24 (m, 1H), 7.28 (d, 1H), 7.36 (t, 2H), 7.56 (d, 1H), 7.71 (t, 1H), 7.82-7.91 (m, 4H), 8.46 (d, 1H), 9.10 (t, 1H). UPLC-MS (ESI+): [M + H]⁺ = 570. | from F.1 according to GP 9.1 |
| 23 | 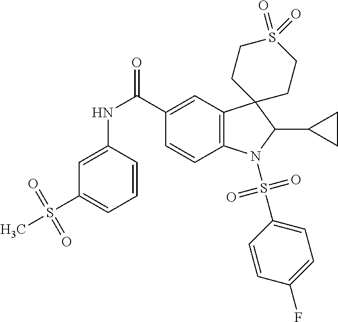 [SMILES: CS(=O)(=O)c1cccc(NC(=O)c2ccc3c(c2)C2(CCS(=O)(=O)CC2)C(C2CC2)N3S(=O)(=O)c2ccc(F)cc2)c1] | 2-cyclopropyl-1-[(4- fluorophenyl) sulfonyl]-N-(3- mesylphenyl)- 1,2,2′,3′,5′,6′- hexahydrospiro[indole- 3,4′-thiopyran]- 5-carboxannide 1′,1′-dioxide | ¹H-NMR (400 MHz, DMSO-d6): Shift [ppm] = 0.30 (d, 1H), 0.34- 0.44 (m, 1H), 0.44-0.53 (m, 1H), 0.53-0.62 (m, 1H), 0.77- 1.04 (m, 3H), 1.13-1.17 (m, 1H), 1.48-1.55 (m, 1H), 2.59- 2.65 (m, 2H), 3.18 (s, 3H), 3.43- 3.53 (m, 1H), 3.59-3.66 (m, 1H), 4.37 (d, 1H), 7.36-7.41 (m, 2H), 7.61-7.64 (m, 3H), 7.87-7.96 (m, 4H), 8.05-8.09 (m, 1H), 8.30 (s, 1H), 10.47 (s, 1H). UPLC-MS (ESI+): [M + H]⁺ = 633. | from F.1 according to GP 9.1 |
| 23.1 | Enantiomer 1 of Ex. 23 | | Rₜ = 6.94 min | HPLC |
| 23.2 | Enantiomer 2 of Ex. 23 | | Rₜ = 8.60 min | method |
| | | | | A with |
| | | | | Column: |
| | | | | Chiralcel |
| | | | | OZ-H |
| | | | | 5 μm |
| | | | | 150 × 4.6 |
| | | | | mm; Solvent: |
| | | | | ethanol/ |
| | | | | methanol 1:1 |
| | | | | (v/v); |
| | | | | Detection: |
| | | | | DAD 280 nm |
| 24 | 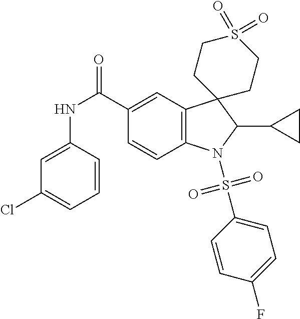 [SMILES: O=C(Nc1cccc(Cl)c1)c1ccc2c(c1)C1(CCS(=O)(=O)CC1)C(C1CC1)N2S(=O)(=O)c1ccc(F)cc1] | N-(3-chlorophenyl)- 2-cyclopropyl-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.25 (d, 1H), 0.34- 0.43 (m, 1H), 0.43-0.51 (m, 1H), 0.51-0.62 (m, 1H), 0.78- 0.86 (m, 2H), 0.92-1.03 (m, 1H), 1.49 (dt, 1H), 2.54-2.69 (m, 2H), 3.11-3.25 (m, 2H), 3.62 (dt, 1H), 4.37 (d, 1H), 7.13 (dd, 1H), 7.32-7.44 (m, 4H), 7.59- 7.66 (m, 2H), 7.81-7.93 (m, 4H), 10.27 (s, 1H). UPLC-MS (ESI+): [M + H]⁺ = 589/591 (Cl isotope pattern). | from F.1 according to GP 9.1 |
| 25 | 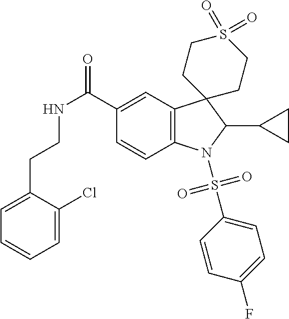 [SMILES: O=C(NCCc1ccccc1Cl)c1ccc2c(c1)C1(CCS(=O)(=O)CC1)C(C1CC1)N2S(=O)(=O)c1ccc(F)cc1] | N-[2-(2- chlorophenyl)ethyl]- 2-cyclopropyl-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.22 (d, 1H), 0.31- 0.41 (m, 1H), 0.43-0.50 (m, 1H), 0.52-0.59 (m, 1H), 0.76- 0.86 (m, 1H), 0.90-1.00 (m, 1H), 1.16-1.27 (m, 1H), 1.41 (dt, 1H), 2.50-2.60 (m, 2H), 2.91 (t, 2H), 3.14-3.22 (m, 2H), 3.40-3.47 (m, 2H), 3.53-3.65 (m, 1H), 4.31 (d, 1H), 7.18-7.41 (m, 6H), 7.53 (d, 1H), 7.67 (d, 1H), 7.78 (dd, 1H), 7.83-7.87 (m, 2H), 8.59 (t, 1H). UPLC-MS (ESI+): [M + H]⁺ = 617/619 (Cl isotope pattern). | from F.1 according to GP 9.1 |
| 26 | 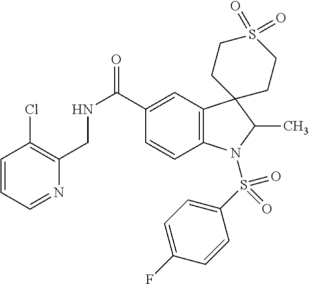 [SMILES: CC1N(S(=O)(=O)c2ccc(F)cc2)c2ccc(C(=O)NCc3ncccc3Cl)cc2C12CCS(=O)(=O)CC2] | N-[(3-chloropyridin- 2-yl)methyl]-1-[(4- fluorophenyl)sulfonyl]- 2-methyl- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.30 (d, 1H), 1.25 (d, 3H), 1.49 (dt, 1H), 2.12 (d, 1H), 2.62 (d, 1H), 3.06-3.16 (m, 2H), 3.40-3.54 (m, 2H), 4.63 (d, 2H), 4.76 (q, 1H), 7.32 (dd, 1H), 7.37-7.42 (m, 2H), 7.56 (d, 1H), 7.80-7.94 (m, 5H), 8.44 (dd, 1H), 8.93 (t, 1H). UPLC-MS (ESI+): [M + H]⁺ = 578/580 (Cl isotope pattern). | from F.2 according to GP 9.1 |
| 27 | 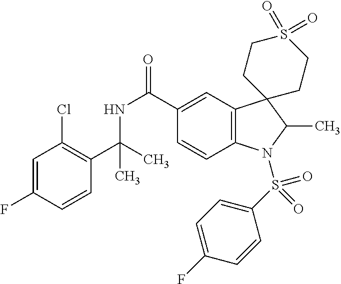 [SMILES: CC1N(S(=O)(=O)c2ccc(F)cc2)c2ccc(C(=O)NC(C)(C)c3ccc(F)cc3Cl)cc2C12CCS(=O)(=O)CC2] | N-(2-chloro-4- fluoro-α,α- dimethylbenzyl)-1- [(4-fluorophenyl) sulfonyl]- 2-methyl- 1,2,2′,3′,5′,6′- hexahydrospiro [indole-3,4′-thiopyran]- 5-carboxamide 1′,1′-dioxide | ¹H-NMR (300 MHz, DMSO-d6): Shift [ppm] = 0.29 (d, 1H), 1.24 (d, 3H), 1.50 (dt, 1H), 1.72 (s, 6H), 2.03-2.14 (m, 1H), 2.50- 2.65 (m, 2H), 3.07-3.13 (m, 2H), 3.49 (dt, 1H), 4.75 (q, 1H), 7.14 (dt, 1H), 7.21 (dd, 1H), 7.36- 7.42 (m, 2H), 7.49 (d, 1H), 7.53 (dd, 1H), 7.69 (d, 1H), 7.74 (dd, 1H), 7.89-7.93 (m, 2H), 8.50 (s, 1H). UPLC-MS (ESI+): [M + H]⁺ = 623/625 (Cl isotope pattern). | from F.2 according to GP 9.1 |
| 28 | 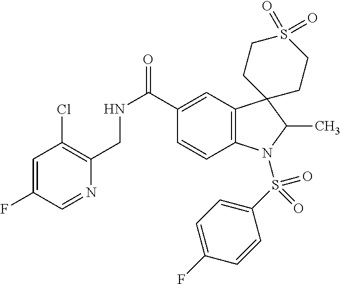 [SMILES: CC1N(S(=O)(=O)c2ccc(F)cc2)c2ccc(C(=O)NCc3ncc(F)cc3Cl)cc2C12CCS(=O)(=O)CC2] | N-[(3-chloro-5- fluoropyridin-2- yl)methyl]-1-[(4- fluorophenyl) sulfonyl]-2-methyl- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.32 (d, 1H), 1.24 (d, 3H), 1.48 (dt, 1H), 2.08-2.13 (m, 1H), 2.56-2.69 (m, 2H), 3.06-3.14 (m, 3H), 4.55 (d, 2H), 4.74 (q, 1H), 7.35-7.41 (m, 2H), 7.53 (d, 1H), 7.75 (s, 1H), 7.82 (dd, 1H), 7.89-7.93 (m, 2H), 8.00 (dd, 1H), 8.41 (d, 1H), 8.98 (t, 1H). UPLC-MS (ESI+): [M + H]⁺ = 596/598 (Cl isotope pattern). | from F.2 according to GP 9.1 |
| 29 | 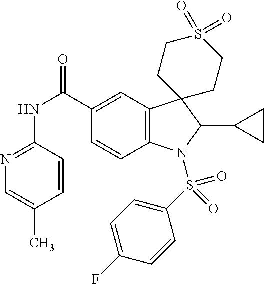 | 2-cyclopropyl-1-[(4- fluorophenyl) sulfonyl]-N-(5- methylpyridin-2-yl)- 1,2,2′,3′,5′,6′- hexahydrospiro[indole- 3,4′-thiopyran]- 5-carboxamide 1′,1′-dioxide | 1H-NMR (300 MHz, DMSO-d6): Shift [ppm] = 0.29 (d, 1H), 0.34- 0.44 (m, 1H), 0.45-0.53 (m, 1H), 0.53-0.62 (m, 1H), 0.76- 0.87 (m, 1H), 0.92-1.03 (m, 1H), 1.53 (dt, 1H), 2.24 (s, 3H), 3.15-3.23 (m, 2H), 3.58 (dt, 1H), 4.33 (d, 1H), 7.38 (t, 2H), 7.54-7.64 (m, 2H), 7.83-8.06 (m, 5H), 8.17 (dbr, 1H), 10.72 (s, 1H). UPLC-MS (ESI+): [M + H]⁺ = 660. | from F.1 according to GP 9.2 |
| 30 | 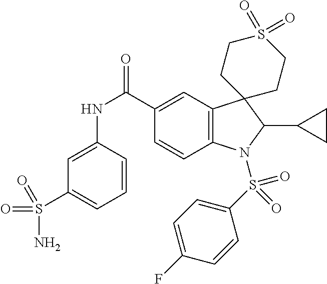 [SMILES: NS(=O)(=O)c1cccc(NC(=O)c2ccc3c(c2)C2(CCS(=O)(=O)CC2)C(C2CC2)N3S(=O)(=O)c2ccc(F)cc2)c1] | 2-cyclopropyl-1-[(4- fluorophenyl) sulfonyl]-N-(3- sulfamoylphenyl)- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.29 (d, 1H), 0.35- 0.44 (m, 1H), 0.44-0.53 (m, 1H), 0.53-0.64 (m, 1H), 0.77- 0.87 (m, 1H), 0.93-1.04 (m, 1H), 1.51 (dt, 1H), 3.62 (dt, 1H), 4.37 (d, 1H), 7.30-7.43 (m, 3H), 7.49-7.55 (m, 2H), 7.61 (d, 1H), 7.84-7.99 (m, 5H), 8.23 (br. s., 1H), 10.39 (s, 1H). UPLC-MS (ESI+): [M + H]⁺ = 634. | from F.1 according to GP 9.2 |
| 30.1 | Enantiomer 1 of Ex. 30 | | Rₜ = 6.47 min | HPLC method |
| 30.2 | Enantiomer 2 of Ex. 30 | | Rₜ = 7.65 min | A with |
| | | | | Column: |
| | | | | Chiralpak IB |
| | | | | 5 μm |
| | | | | 150 × 4.6 |
| | | | | mm; Solvent |
| | | | | Hexan/ |
| | | | | Ethanol 50:50 |
| | | | | (v/v); |
| | | | | Detection: |
| | | | | DAD 280 nm |
| 31 | 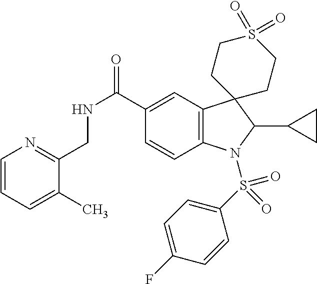 [SMILES: Cc1cccnc1CNC(=O)c1ccc2c(c1)C1(CCS(=O)(=O)CC1)C(C1CC1)N2S(=O)(=O)c1ccc(F)cc1] | 2-cyclopropyl-1-[(4- fluorophenyl) sulfonyl]-N-[(3- methylpyridin-2- yl)methyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.20 (d, 1H), 0.31- 0.42 (m, 1H), 0.42-0.51 (m, 1H), 0.51-0.61 (m, 1H), 0.75- 0.87 (m, 1H), 0.89-1.02 (m, 1H), 1.42 (dt, 1H), 2.30 (s, 3H), 3.13-3.23 (m, 2H), 3.59 (dt, 1H), 4.32 (d, 1H), 4.53 (d, 2H), 7.16 (dt, 1H), 7.32-7.41 (m, 2H), 7.50-7.57 (m, 2H), 7.78- 7.91 (m, 4H), 8.29 (mc, 1H), 8.86 (t, 1H). UPLC-MS (ESI+): [M + H]⁺ = 584. | from F.1 according to GP 9.1 |
| 31.1 | Enantiomer 1 of Ex. 31 | | Rₜ = 13.53 min | HPLC |
| 31.2 | Enantiomer 2 of Ex. 31 | | Rₜ = 17.70 min | method |
| | | | | B1 with |
| | | | | Column: |
| | | | | Chiralpak IA |
| | | | | 5 μm |
| | | | | 150 × 4.6 |
| | | | | mm; Solvent |
| | | | | Hexan/2- |
| | | | | Propanol/ |
| | | | | Diethylamine |
| | | | | 70:30:0.1 |
| | | | | (v/v/v) |
| 32 | 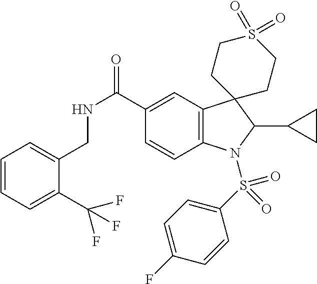 [SMILES: O=C(NCc1ccccc1C(F)(F)F)c1ccc2c(c1)C1(CCS(=O)(=O)CC1)C(C1CC1)N2S(=O)(=O)c1ccc(F)cc1] | 2-cyclopropyl-1-[(4- fluorophenyl) sulfonyl]-N-[2- (trifluoromethyl) benzyl]-1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.25 (d, 1H), 0.33- 0.44 (m, 1H), 0.44-0.51 (m, 1H), 0.51-0.62 (m, 1H), 0.76- 0.87 (m, 1H), 0.91-1.03 (m, 1H), 1.45 (dt, 1H), 3.14-3.24 (m, 2H), 3.61 (dt, 1H), 4.34 (d, 1H), 4.61 (d, 2H), 7.33-7.94 (m, 11H), 9.08 (t, 1H). UPLC-MS (ESI+): [M + H]⁺ = 637. | from F.1 according to GP 9.1 |
| 32.1 | Enantiomer 1 of Ex. 32 | | Rₜ = 4.37 min | HPLC method |
| 32.2 | Enantiomer 2 of Ex. 32 | | Rₜ = 7.00 min | B2 with |
| | | | | column |
| | | | | Chiralpak |
| | | | | AD-H |
| | | | | 5 μm |
| | | | | 150 × 4.6 mm; |
| | | | | Solvent: |
| | | | | Hexan/ |
| | | | | 2-Propanol |
| | | | | 70:30 (v/v); |
| | | | | Detection: |
| | | | | DAD 280 nm |
| 33 | 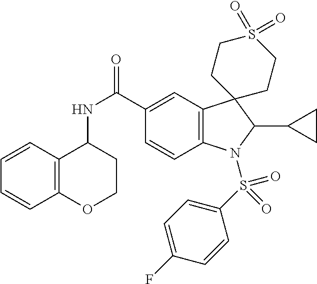 [SMILES: O=C(NC1CCOc2ccccc21)c1ccc2c(c1)C1(CCS(=O)(=O)CC1)C(C1CC1)N2S(=O)(=O)c1ccc(F)cc1] | 2-cyclopropyl-N- (3,4-dihydro-2H- chromen-4-yl)-1- [(4-fluorophenyl) sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.20 (d, 1H), 0.33- 0.41 (m, 1H), 0.42-0.50 (m, 1H), 0.51-0.60 (m, 1H), 0.77- 0.85 (m, 1H), 0.91-1.01 (m, 1H), 1.43 (dt, 1H), 1.92-2.11 (m, 1H), 3.12-3.20 (m, 2H), 3.59 (dt, 1H), 4.16-4.28 (m, 2H), 4.33 (dd, 1H), 5.19-5.28 (m, 1H), 6.76 (d, 1H), 6.81-6.87 (m, 1H), 7.08-7.15 (m, 2H), 7.37 (dt, 2H), 7.55 (d, 1H), 7.74-7.94 (m, 4H), 8.81 (d, 1H). UPLC-MS (ESI+): [M + H]⁺ = 611. | from F.1 according to Gp 9.1 |
| 33.1 | Diastereomers 1 and 2, | | Rₜ = 3.64 min | HPLC method |
| | 1:1) of Ex. 33 | | | B3 with |
| 33.2 | Diastereomer 3 of Ex. 33 | | Rₜ = 4.31 min | column |
| 33.3 | Diastereomer 4 of Ex. 33 | | Rₜ = 5.39 min | Chiralpak IC |
| | | | | 5 μm |
| | | | | 150 × 4.6 |
| | | | | mm; Solvent |
| | | | | Methanol |
| 34 | 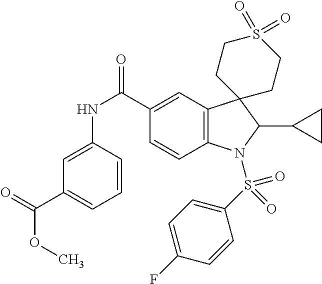 [SMILES: COC(=O)c1cccc(NC(=O)c2ccc3c(c2)C2(CCS(=O)(=O)CC2)C(C2CC2)N3S(=O)(=O)c2ccc(F)cc2)c1] | methyl 3-[({2- cyclopropyl-1-[(4- fluorophenyl)sulfonyl]- 1′,1′-dioxido- 1,2,2′,3′,5′,6′- hexahydrospiro[indole- 3,4′-thiopyran]- 5-yl}carbonyl) amino]benzoate | ¹H-NMR (300 MHz, DMSO-d6): Shift [ppm] = 0.29 (d, 1H), 0.35- 0.44 (m, 1H), 0.45-0.53 (m, 1H), 0.53-0.62 (m, 1H), 0.79- 0.87 (m, 1H), 0.95-1.03 (m, 1H), 1.51 (dt, 1H), 2.56-2.66 (m, 3H), 3.17-3.24 (m, 2H), 3.62 (dt, 1H), 3.84 (s, 3H), 4.37 (d, 1H), 7.39 (t br, 2H), 7.48 (t, 1H), 7.61 (d, 1H), 7.66 (d, 1H), 7.85-8.04 (m, 6H), 8.33 (mc, 1H), 10.32 (s, 1H). UPLC-MS (ESI+): [M + H]⁺ = 613. | from F.1 according to GP 9.1 |
| 34.1 | Enantiomer 1 of Ex. 34 | | Rₜ = 13.62 min | HPLC |
| 34.2 | Enantiomer 2 of Ex. 34 | | Rₜ = 17.61 min | method |
| | | | | B1 with |
| | | | | column |
| | | | | Chiralpak IA |
| | | | | 5 μm |
| | | | | 150 × 4.6 |
| | | | | mm; Solvent |
| | | | | Hexan/ |
| | | | | Ethanol 70:30 |
| | | | | (v/v) |
| 35 | 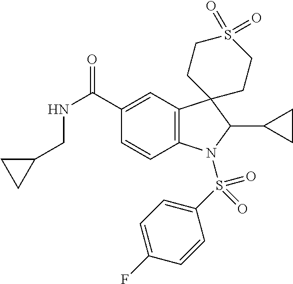 [SMILES: O=C(NCC1CC1)c1ccc2c(c1)C1(CCS(=O)(=O)CC1)C(C1CC1)N2S(=O)(=O)c1ccc(F)cc1] | 2-cyclopropyl-N- (cyclopropylmethyl)- 1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.19-0.28 (m, 3H), 0.38-0.64 (m, 5H), 0.81-0.88 (m, 1H), 0.97-1.05 (m, 2H), 1.47 (dt, 1H), 2.44-2.63 (m, 3H), 3.07-3.18 (m, 2H), 3.20- 3.23 (m, 2H), 3.63 (dt, 1H), 4.36 (d, 1H), 7.37-7.43 (m, 2H), 7.57 (d, 1H), 7.76 (d, 1H), 7.84-7.91 (m, 3H), 8.56 (t, 1H). UPLC-MS (ESI+): [M + H]⁺ = 533. | from F.1 according to GP 9.2 |
| 35.1 | Enantiomer 1 of Ex. 35 | | Rₜ = 3.20 min | HPLC |
| 35.2 | Enantiomer 2 of Ex. 35 | | Rₜ = 3.76 min | method |
| | | | | B3 with |
| | | | | column |
| | | | | Chiralpak IC |
| | | | | 5 μm |
| | | | | 150 × 4.6 |
| | | | | mm; Solvent |
| | | | | Methanol |
| 36 | 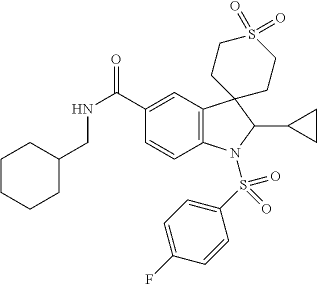 [SMILES: O=C(NCC1CCCCC1)c1ccc2c(c1)C1(CCS(=O)(=O)CC1)C(C1CC1)N2S(=O)(=O)c1ccc(F)cc1] | N- (cyclohexylmethyl)- 2-cyclopropyl-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.22 (d, 1H), 0.31- 0.42 (m, 1H), 0.42-0.50 (m, 1H), 0.50-0.62 (m, 1H), 2.98- 3.10 (m, 2H), 3.13-3.23 (m, 2H), 3.59 (dt, 1H), 4.31 (d, 1H), 7.36 (t, 2H), 7.52 (d, 1H), 7.71 (mc, 1H), 7.77-7.90 (m, 3H), 8.39 (t, 1H). UPLC-MS (ESI+): [M + H]⁺ = 575. | from F.1 according to GP 9.2 |
| 36.1 | Enantiomer 1 of Ex. 34 | | Rₜ = 6.28 min | HPLC method |
| 36.2 | Enantiomer 2 of Ex. 34 | | Rₜ = 8.31 min | B2 with |
| | | | | column |
| | | | | Chiralpak IA |
| | | | | 5 μm |
| | | | | 150 × 4.6 |
| | | | | mm; Solvent: |
| | | | | Hexan/2- |
| | | | | Propanol 70:30 |
| | | | | (v/v); |
| | | | | Detection: |
| | | | | DAD 280 nm |
| 37 | 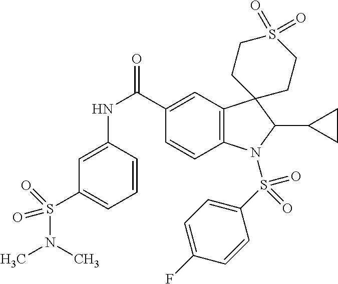 [SMILES: CN(C)S(=O)(=O)c1cccc(NC(=O)c2ccc3c(c2)C2(CCS(=O)(=O)CC2)C(C2CC2)N3S(=O)(=O)c2ccc(F)cc2)c1] | 2-cyclopropyl-N-[3- (N,N- dimethylsulfamoyl) phenyl]-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.29 (d, 1H), 0.35- 0.44 (m, 1H), 0.44-0.53 (m, 1H), 0.53-0.64 (m, 1H), 0.77- 0.89 (m, 1H), 0.91-1.04 (m, 1H), 1.51 (dt, 1H), 2,61 (s, 3H), 3.62 (dt, 1H), 4.37 (d, 1H), 7.33- 7.46 (m, 3H), 7.55-7.65 (m, 2H), 7.82-8.18 (m, 6H), 10.44 (s, 1H). UPLC-MS (ESI+): [M + H]⁺ = 662. | from F.1 according to GP 9.2 |
| 37.1 | Enantiomer 1 of Ex. 37 | | Rₜ: = 12.79 min | HPLC |
| 37.2 | Enantiomer 2 of Ex. 37 | | Rₜ: = 14.76 min | method |
| | | | | A with |
| | | | | Column: |
| | | | | Chiralpak IB |
| | | | | 5 μm |
| | | | | 150 × 4.6 |
| | | | | mm; Solvent |
| | | | | Hexan/ |
| | | | | Ethanol 70:30 |
| | | | | (v/v); |
| | | | | Detection: |
| | | | | DAD 280 nm |
| 38 | 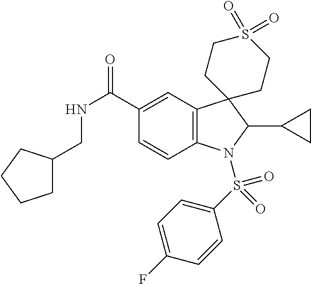 [SMILES: O=C(NCC1CCCC1)c1ccc2c(c1)C1(CCS(=O)(=O)CC1)C(C1CC1)N2S(=O)(=O)c1ccc(F)cc1] | N- (cyclopentylmethyl)- 2-cyclopropyl-1- [(4-fluorophenyl) sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.23 (d, 1H), 0.31- 0.42 (m, 1H), 0.42-0.50 (m, 1H), 0.50-0.61 (m, 1H), 0.76- 0.86 (m, 1H), 0.90-1.01 (m, 1H), 3.06-3.21 (m, 4H), 3.59 (dt, 1H), 4.31 (d, 1H), 7.36 (t, 2H), 7.52 (d, 1H), 7.70 (m, 1H), 7.74-7.93 (m, 3H), 8.43 (t, 1H). UPLC-MS (ESI+): [M + H]⁺ = 561. | from F.1 according to GP 9.1 |
| 38.1 | Enantiomer 1 of Ex. 38 | | Rₜ = 5.87 min | HPLC |
| 38.2 | Enantiomer 2 of Ex. 38 | | Rₜ = 8.01 min | method |
| | | | | B1 with |
| | | | | column |
| | | | | Chiralpak IA |
| | | | | 5 μm |
| | | | | 150 × 4.6 |
| | | | | mm; Solvent |
| | | | | Hexan/ |
| | | | | Ethanol 70:30 |
| | | | | (v/v) |
| 39 | 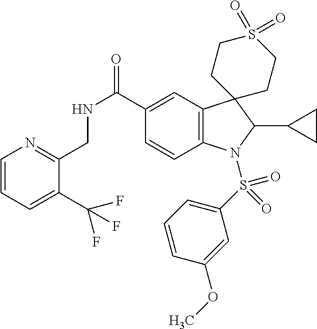 [SMILES: COc1cccc(S(=O)(=O)N2c3ccc(C(=O)NCc4ncccc4C(F)(F)F)cc3C3(CCS(=O)(=O)CC3)C2C2CC2)c1] | 2-cyclopropyl-1-[(3- methoxyphenyl) sulfonyl]-N-{[3- (trifluoromethyl) pyridin-2-yl]methyl}- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.16 (d, 1H), 0.32- 0.42 (m, 1H), 0.43-0.50 (m, 1H), 0.51-0.60 (m, 1H), 0.79- 0.89 (m, 1H), 0.90-1.03 (m, 1H), 1.39 (dt, 1H), 3.14-3.22 (m, 2H), 3.61 (dt, 1H), 3.73 (s, 3H), 4.34 (d, 1H), 4.69 (d, 2H), 7.14-7.22 (m, 1H), 7.26-7.32 (m, 2H), 7.38-7.62 (m, 3H), 7.57 (d, 1H), 7.77-7.88 (m, 2H), 8.10-8.17 (m, 1H), 8.76 (d, 1H), 8.99 (t, 1H). UPLC-MS (ESI+): [M + H]⁺ = 650. | from F.4 according to GP 9.1 |
| 39.1 | Enantiomer 1 of Ex. 39 | | Rₜ = 10.36 min | HPLC |
| 39.2 | Enantiomer 2 of Ex. 39 | | Rₜ = 13.29 min | method |
| | | | | B2 with |
| | | | | column |
| | | | | Chiralpak IA |
| | | | | 5 μm |
| | | | | 150 × 4.6 |
| | | | | mm; Solvent: |
| | | | | Hexan/2- |
| | | | | Propanol |
| | | | | 70:30 (v/v); |
| | | | | Detection: |
| | | | | DAD 254 nm |
| 40 | 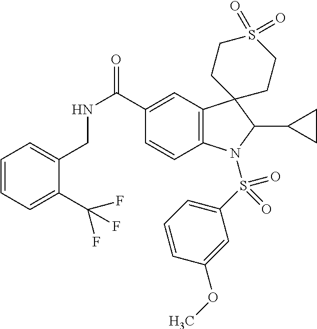 [SMILES: COc1cccc(S(=O)(=O)N2c3ccc(C(=O)NCc4ccccc4C(F)(F)F)cc3C3(CCS(=O)(=O)CC3)C2C2CC2)c1] | 2-cyclopropyl-1-[(3- methoxyphenyl) sulfonyl]-N-[2- (trifluoromethyl) benzyl]-1,2,2′,3′,5′,6′- hexahydrospiro [indole-3,4′-thiopyran]- 5-carboxamide 1′,1′-dioxide | ¹H-NMR (300 MHz, DMSO-d6): Shift [ppm] = 0.18 (d, 1H), 0.32- 0.44 (m, 1H), 0.44-0.62 (m, 2H), 0.79-0.90 (m, 1H), 0.90- 1.02 (m, 1H), 1.41 (dt, 1H), 3.13- 3.23 (m, 2H), 3.61 (dt, 1H), 3.73 (s, 3H), 4.35 (d, 1H), 4.61 (d, 2H), 7.15-7.22 (m, 1H), 7.26- 7.33 (m, 2H), 7.38-7.65 (m, 5H), 7.69 (d, 1H), 7.77-7.92 (m, 2H), 9.05 (t, 1H). UPLC-MS (ESI+): [M + H]⁺ = 649. | from F.4 according to GP 9.1 |
| 40.1 | Enantiomer 1 of Ex. 40 | | Rₜ = 5.57 min | HPLC |
| 40.2 | Enantiomer 2 of Ex. 40 | | Rₜ = 7.40 min | method |
| | | | | B2 with |
| | | | | column |
| | | | | Chiralpak IA |
| | | | | 5 μm |
| | | | | 150 × 4.6 |
| | | | | mm; Solvent: |
| | | | | Hexan/2- |
| | | | | Propanol |
| | | | | 70:30 (v/v); |
| | | | | Detection: |
| | | | | DAD 280 nm |
| 41 | 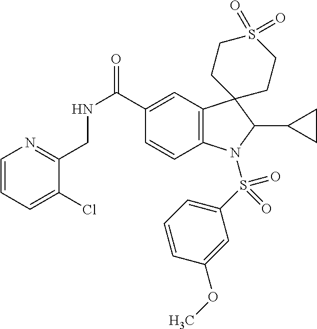 [SMILES: COc1cccc(S(=O)(=O)N2c3ccc(C(=O)NCc4ncccc4Cl)cc3C3(CCS(=O)(=O)CC3)C2C2CC2)c1] | N-[(3-chloropyridin- 2-yl)methyl]-2- cyclopropyl-1-[(3- methoxyphenyl) sulfonyl]-1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.16-0.21 (m, 1H), 0.37-0.62 (m, 3H), 0.84-0.90 (m, 1 H), 0.94-1.02 (m, 1H), 1.42 (dt, 1H), 2.42-2.62 (m, 3H), 3.21-3.22 (m, 2H), 3.65 (dt, 1H), 3.77 (s, 3H), 4.37 (d, 1H), 4.67 (d, 2H), 7.20-7.23 (m, 1H), 7.31-7.38 (m, 3H), 7.43- 7.49 (m, 1H), 7.60 (d, 1H), 7.82 (d, 1H), 7.87-7.94 (m, 2H), 8.49 (dd, 1H), 8.96 (t, 1H). UPLC-MS (ESI+): [M + H]⁺ = 616/618 (Cl isotope pattern). | from F.4 according to GP 9.1 |
| 41.1 | Enantiomer 1 of Ex. 41 | | Rₜ = 20.28 min | HPLC |
| 41.2 | Enantiomer 2 of Ex. 41 | | Rₜ = 24.87 min | method |
| | | | | A with |
| | | | | column: |
| | | | | Chiralpak IA |
| | | | | 5 μm |
| | | | | 150 × 4.6 |
| | | | | mm; Solvent |
| | | | | Hexan/2- |
| | | | | Propanol |
| | | | | 70:30 (v/v); |
| | | | | Detection: |
| | | | | DAD 280 nm |
| 42 | 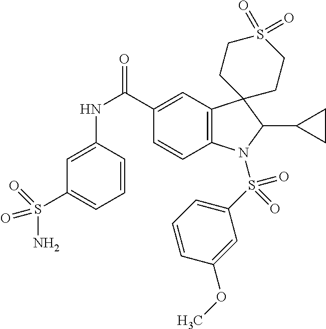 [SMILES: COc1cccc(S(=O)(=O)N2c3ccc(C(=O)Nc4cccc(S(N)(=O)=O)c4)cc3C3(CCS(=O)(=O)CC3)C2C2CC2)c1] | 2-cyclopropyl-1-[(3- methoxyphenyl) sulfonyl]-N-(3- sulfamoylphenyl) 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.16-0.29 (m, 1H), 0.37-0.48 (m, 1H), 0.48-0.67 (m, 2H), 0.84-0.95 (m, 1H), 0.95-1.07 (m, 1H), 1.49 (dt, 1H), 3.67 (dt, 1H), 3.77 (s, 3H), 4.41 (d, 1H), 7.19-7.65 (m, 8H), 7.84-8.02 (m, 3H), 8.27 (s, 1H), 10.43 (s, 1H). UPLC-MS (ESI+): [M + H]⁺ = 646. | from F.4 according to GP 9.1 |
| 42.1 | Enantiomer 1 of Ex. 42 | | Rₜ = 2.91 min | HPLC |
| 42.2 | Enantiomer 2 of Ex. 42 | | Rₜ = 3.42 min | method |
| | | | | A with |
| | | | | column: |
| | | | | Chiralpak IC |
| | | | | 5 μm |
| | | | | 150 × 4.6 |
| | | | | mm; Solvent |
| | | | | Methanol; |
| | | | | Detection: |
| | | | | DAD 280 nm |
| 43 | 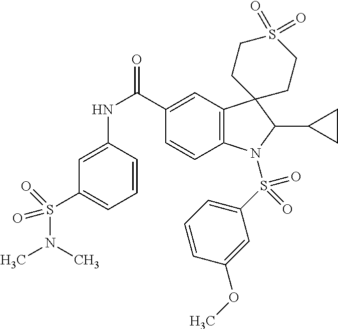 [SMILES: COc1cccc(S(=O)(=O)N2c3ccc(C(=O)Nc4cccc(S(=O)(=O)N(C)C)c4)cc3C3(CCS(=O)(=O)CC3)C2C2CC2)c1] | 2-cyclopropyl-N-[3- (N,N- dimethylsulfamoyl) phenyl]-1-[(3- methoxyphenyl) sulfonyl]-1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.19-0.25 (m, 1H), 0.38-0.65 (m, 3H), 0.85- 1.03 (m, 2H), 1.49 (dt, 1H), 2.58- 2.68 (m, 9H), 3.23-3.25 (m, 2H), 3.67 (dt, 1H), 3.77 (s, 3H), 4.42 (d, 1H), 7.21-7.25 (m, 1H), 7.33-7.37 (m, 2H), 7.45-7.50 (m, 2H), 7.61-7.68 (m, 2H), 7.87 (d, 1H), 7.99 (dd, 1H), 8.06- 8.09 (m, 1H), 8.18-8.19 (m, 1H), 10.48 (s, 1H). UPLC-MS (ESI+): [M + H]⁺ = 674. | from F.4 according to GP 9.1 |
| 43.1 | Enantiomer 1 of Ex. 43 | | Rₜ = 4.63 min | HPLC |
| 43.2 | Enantiomer 2 of Ex. 43 | | Rₜ = 6.92 min | method |
| | | | | B2 with |
| | | | | column |
| | | | | Chiralpak IC |
| | | | | 5 μm |
| | | | | 150 × 4.6 |
| | | | | mm; Solvent: |
| | | | | Hexan/ |
| | | | | Ethanol 0:100 |
| | | | | (v/v); |
| | | | | Detection: |
| | | | | DAD 280 nm |
| 44 | 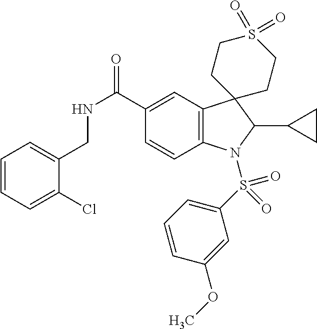 [SMILES: COc1cccc(S(=O)(=O)N2c3ccc(C(=O)NCc4ccccc4Cl)cc3C3(CCS(=O)(=O)CC3)C2C2CC2)c1] | N-(2-chlorobenzyl)- 2-cyclopropyl-1-[(3- methoxyphenyl) sulfonyl]-1,2,2′,3′,5′,6′- hexahydrospiro [indole-3,4′-thiopyran]- 5-carboxamide 1′,1′-dioxide | ¹H-NMR (300 MHz, DMSO-d6): Shift [ppm] = 0.18-0.23 (m, 1H), 0.38-0.64 (m, 3H), 0.82-0.92 (m, 1H), 0.93-1.03 (m, 1H), 1.44 (dt, 1H), 2.44-2.63 (m, 3H), 3.21-3.22 (m, 2H), 3.65 (dt, 1H), 3.77 (s, 3H), 4.37 (d, 1H), 4.53 (d, 2H), 7.20-7.23 (m, 1H), 7.26-7.38 (m, 5H), 7.43- 7.49 (m, 2H), 7.61 (d, 1H), 7.82 (d, 1H), 7.91 (dd, 1H), 9.02 (t, 1H). UPLC-MS (ESI+): [M + H]⁺ = 615/617 (Cl isotope pattern). | from F4 according to GP 9.1 |
| 44.1 | Enantiomer 1 of Ex. 44 | | Rₜ = 8.69 min | HPLC |
| 44.2 | Enantiomer 2 of Ex. 44 | | Rₜ = 12.51 min | method |
| | | | | A with |
| | | | | Column: |
| | | | | Chiralpak IA |
| | | | | 5 μm |
| | | | | 150 × 4.6 |
| | | | | mm; Solvent |
| | | | | Hexan/ |
| | | | | Ethanol 70:30 |
| | | | | (v/v); |
| | | | | Detection: |
| | | | | DAD 280 nm |
| 45 | 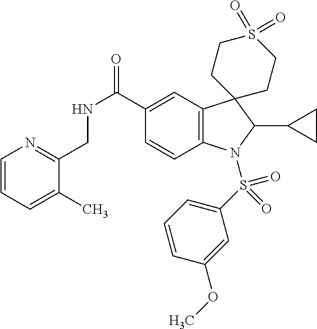 [SMILES: COc1cccc(S(=O)(=O)N2c3ccc(C(=O)NCc4ncccc4C)cc3C3(CCS(=O)(=O)CC3)C2C2CC2)c1] | 2-cyclopropyl-1-[(3- methoxyphenyl) sulfonyl]-N-[(3- methylpyridin-2-yl) methyl]-1,2,2′,3′,5′,6′- hexahydrospiro [indole-3,4′-thiopyran]- 5-carboxamide 1′,1′-dioxide | ¹H-NMR (300 MHz, DMSO-d6): Shift [ppm] = 0.16-0.20 (m, 1H), 0.36-0.63 (m, 3H), 0.83-0.91 (m, 1H), 0.95-1.02 (m, 1H), 1.42 (dt, 1H), 2.34 (s, 3H), 2.42- 2.63 (m, 3H), 3.20-3.22 (m, 2H), 3.64 (dt, 1H), 3.76 (s, 3H), 4.36 (d, 1H), 4.56 (d, 2H), 7.18- 7.23 (m, 2H), 7.30-7.33 (m, 2H), 7.45 (t, 1H), 7.56-7.60 (m, 2H), 7.82 (d, 1H), 7.89 (dd, 1H), 8.33-8.35 (m, 1H), 8.87 (t, 1H). UPLC-MS (ESI+): [M + H]⁺ = 596. | from F.4 according to GP 9.1 |
| 45.1 | Enantiomer 1 of Ex. 45 | | Rₜ = 7.73 min | HPLC |
| 45.2 | Enantiomer 2 of Ex. 45 | | Rₜ = 10.73 min | method |
| | | | | A with |
| | | | | Column: |
| | | | | Chiralpak IA |
| | | | | 5 μm |
| | | | | 150 × 4.6 |
| | | | | mm; Solvent |
| | | | | Hexan/ |
| | | | | Ethanol 50:50 |
| | | | | (v/v); |
| | | | | Detection: |
| | | | | DAD 280 nm |
| 46 | 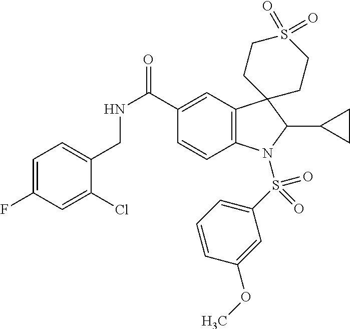 [SMILES: COc1cccc(S(=O)(=O)N2c3ccc(C(=O)NCc4ccc(F)cc4Cl)cc3C3(CCS(=O)(=O)CC3)C2C2CC2)c1] | N-(2-chloro-4- fluorobenzyl)-2- cyclopropyl-1-[(3- methoxyphenyl) sulfonyl]-1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.18-0.22 (m, 1H), 0.38-0.44 (m, 1H), 0.48-0.54 (m, 1H), 0.56-0.62 (m, 1H), 0.85-0.91 (m, 1H), 0.94-1.01 (m, 1H), 1.43 (dt, 1H), 2.44- 2.61 (m, 3H), 3.21-3.22 (m, 2H), 3.65 (dt, 1H), 3.76 (s, 3H), 4.38 (d, 1H), 4.49 (d, 2H), 7.18- 7.23 (m, 2H), 7.31-7.34 (m, 2H), 7.39-7.48 (m, 3H), 7.61 (d, 1H), 7.81 (d, 1H), 7.90 (dd, 1H), 9.01 (t, 1H). UPLC-MS (ESI+): [M + H]⁺ = 633/635 (Cl isotope pattern). | from F.4 according to GP 9.1 |
| 46.1 | Enantiomer 1 of Ex. 46 | | Rₜ = 7.49 min | HPLC |
| 46.2 | Enantiomer 2 of Ex. 46 | | Rₜ = 10.06 min | method |
| | | | | B2 with |
| | | | | column |
| | | | | Chiralpak IA |
| | | | | 5 μm |
| | | | | 150 × 4.6 |
| | | | | mm; Solvent: |
| | | | | Hexan/ |
| | | | | Ethanol 70:30 |
| | | | | (v/v); |
| | | | | Detection: |
| | | | | DAD 280 nm |
| 47 | 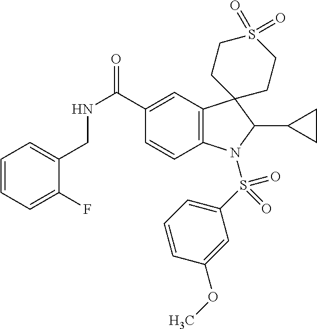 [SMILES: COc1cccc(S(=O)(=O)N2c3ccc(C(=O)NCc4ccccc4F)cc3C3(CCS(=O)(=O)CC3)C2C2CC2)c1] | 2-cyclopropyl-N-(2- fluorobenzyl)-1-[(3- methoxyphenyl) sulfonyl]-1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.16-0.20 (m, 1H), 0.37-0.44 (m, 1H), 0.47-0.53 (m, 1H), 0.55-0.62 (m, 1H), 0.84-0.90 (m, 1H), 0.94-1.01 (m, 1H), 1.42 (dt, 1H), 2.43- 2.60 (m, 3H), 3.20-3.22 (m, 2H), 3.64 (dt, 1H), 3.76 (s, 3H), 4.37 (d, 1H), 4.45-4.54 (m, 2H), 7.14-7.22 (m, 3H), 7.28-7.39 (m, 4H), 7.43-7.47 (m, 1H), 7.60 (d, 1H), 7.79 (d, 1H), 7.89 (dd, 1H), 8.99 (t, 1H). UPLC-MS (ESI+): [M + H]⁺ = 599. | from F.4 according to GP 9.1 |
| 47.1 | Enantiomer 1 of Ex. 47 | | Rₜ = 7.25 min | HPLC |
| 47.2 | Enantiomer 2 of Ex. 47 | | Rₜ = 9.97 min | method |
| | | | | B2 with |
| | | | | column |
| | | | | Chiralpak IA |
| | | | | 5 μm |
| | | | | 150 × 4.6 |
| | | | | mm; Solvent: |
| | | | | Hexan/ |
| | | | | Ethanol 70:30 |
| | | | | (v/v); |
| | | | | Detection: |
| | | | | DAD 280 nm |
| 48 | 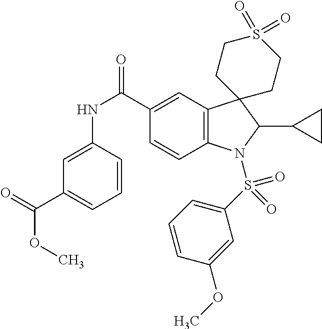 [SMILES: COC(=O)c1cccc(NC(=O)c2ccc3c(c2)C2(CCS(=O)(=O)CC2)C(C2CC2)N3S(=O)(=O)c2cccc(OC)c2)c1] | methyl 3-[({2- cyclopropyl-1-[(3- methoxyphenyl) sulfonyl]-1′,1′-dioxido- 1,2,2′,3′,5′,6′- hexahydrospiro [indole-3,4′- thiopyran]-5- yl}carbonyl)amino] benzoate | ¹H-NMR (400 MHz, DMSO-d6): Shift [ppm] = 0.20-0.25 (m, 1H), 0.39-0.46 (m, 1H), 0.49-0.55 (m, 1H), 0.57-0.63 (m, 1H), 0.86-0.92 (m, 1H), 0.96-1.01 (m, 1H), 1.49 (dt, 1H), 2.44- 2.50 (m, 2H), 2.59-2.65 (m, 1H), 3.23-3.25 (m, 2H), 3.67 (dt, 1H), 3.77 (s, 3H), 3.87 (s, 3H), 4.41 (d, 1H), 7.23 (dd, 1H), 7.33-7.36 (m, 2H), 7.45-7.53 (m, 2H), 7.66 (d, 1H), 7.68-7.71 (m, 1H), 7.88 (d, 1H), 7.98 (dd, 1H), 8.04-8.06 (m, 1H), 8.36- 8.37 (m, 1H), 10.35 (s, 1H). UPLC-MS (ESI+): [M + H]⁺ = 625. | from F.4 according to GP 9.1 |
| 49.1 | 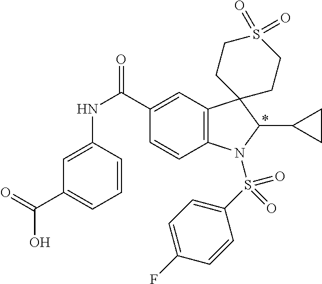 [SMILES: O=C(O)c1cccc(NC(=O)c2ccc3c(c2)C2(CCS(=O)(=O)CC2)C(C2CC2)N3S(=O)(=O)c2ccc(F)cc2)c1] | 3-[({2-cyclopropyl- 1-[(4-fluorophenyl) sulfonyl]- 1′,1′-dioxido- 1,2,2′,3′,5′,6′- hexahydrospiro[indole- 3,4′-thiopyran]- 5-yl}carbonyl) amino]benzoic acid | ¹H-NMR (300 MHz, DMSO-d6): Shift [ppm] = 0.30-0.34 (m, 1H), 0.39-0.63 (m, 3H), 0.83-0.90 (m, 1H), 0.97-1.03 (m, 1H), 1.55 (dt, 1H), 2.50-2.67 (m, 3H), 3.24-3.25 (m, 2H), 3.65 (dt, 1H), 4.40 (d, 1H), 7.39-7.50 (m, 3H), 7.62-7.70 (m, 2H), 7.90-8.02 (m, 5H), 8.32-8.33 (m, 1H), 10.31 (s, 1H), 12.97 (br. s., 1H). UPLC-MS (ESI+): [M + H]⁺ = 599. | prepared by saponification of Ex. 34.1 according to GP7 |
| 49.2 | Enantiomer 2 of Ex. 49.1 | 3-[({2-cyclopropyl- | | prepared by |
| | | 1-[(4-fluorophenyl) | | saponification |
| | | sulfonyl]- | | of Ex. 34.2 |
| | | 1′,1′-dioxido- | | according to |
| | | 1,2,2′,3′,5′,6′- | | GP 7 |
| | | hexahydrospiro[indole- | | |
| | | 3,4′-thiopyran]- | | |
| | | 5-yl}carbonyl) | | |
| | | amino]benzoic acid | | |
| 50 | 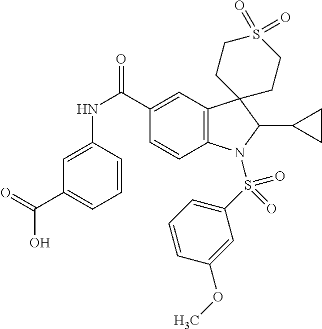 [SMILES: COc1cccc(S(=O)(=O)N2c3ccc(C(=O)Nc4cccc(C(=O)O)c4)cc3C3(CCS(=O)(=O)CC3)C2C2CC2)c1] | 3-[({2-cyclopropyl- 1-[(3-methoxyphenyl) sulfonyl]- 1′,1′-dioxido- 1,2,2′,3′,5′,6′- hexahydrospiro[indole- 3,4′-thiopyran]- 5-yl}carbonyl) amino]benzoic acid | ¹H-NMR (300 MHz, DMSO-d6): Shift [ppm] = 0.20-0.25 (m, 1H), 0.39-0.62 (m, 3H), 0.85- 0.91 (m, 1H), 0.98-1.04 (m, 1H), 1.49 (dt, 1H), 2.50-2.65 (m, 3H), 3.23-3.25 (m, 2H), 3.67 (dt, 1H), 3.77 (s, 3H), 4.41 (d, 1H), 7.23 (dd, 1H), 7.33- 7.36 (m, 2H), 7.45-7.50 (m, 2H), 7.64-7.69 (m, 2H), 7.88 (d, 1H), 7.96-8.02 (m, 2H), 8.33 (mc, 1H), 10.31 (s, 1H), 12.95 (br. s., 1H). UPLC-MS (ESI+): [M + H]⁺ = 611. | prepared by saponification of Ex. 48 according to GP 7 |
| 51.1 | 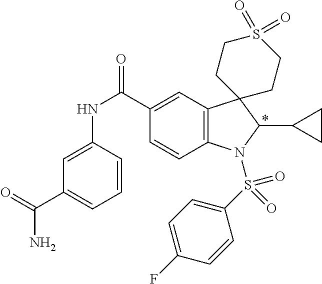 [SMILES: NC(=O)c1cccc(NC(=O)c2ccc3c(c2)C2(CCS(=O)(=O)CC2)C(C2CC2)N3S(=O)(=O)c2ccc(F)cc2)c1] | N-(3- carbamoylphenyl)- 2-cyclopropyl-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.30-0.34 (m, 1H), 0.40-0.46 (m, 1H), 0.49-0.55 (m, 1H), 0.58-0.65 (m, 1H), 0.84-0.89 (m, 1H), 0.99-1.06 (m, 1H), 1.54 (dt, 1H), 2.50- 2.52 (m, 1H), 2.60-2.68 (m, 2H), 3.19-3.28 (m, 2H), 3.65 (dt, 1H), 4.39 (d, 1H), 7.34 (br. s., 1H), 7.40-7.44 (m, 3H), 7.60 (d, 1H), 7.64 (d, 1H), 7.90-7.94 (m, 5H), 7.98 (dd, 1H), 8.15 (mc, 1H), 10.28 (s, 1H). UPLC-MS (ESI+): [M + H]⁺ = 598. | prepared by amide coupling of Ex. 49.1 with NH₃ according to GP 9.2 |
| 51.2 | Enantiomer 2 of Ex. 51.1 | N-(3- | | prepared by |
| | | carbamoylphenyl)- | | amide |
| | | 2-cyclopropyl-1-[(4- | | coupling of |
| | | fluorophenyl)sulfonyl]- | | Ex. 49.2 |
| | | 1,2,2′,3′,5′,6′- | | with NH₃ |
| | | hexahydrospiro[indole- | | according to |
| | | 3,4′-thiopyran]- | | GP 9.2 |
| | | 5-carboxamide | | |
| | | 1′,1′-dioxide | | |
| 52 | 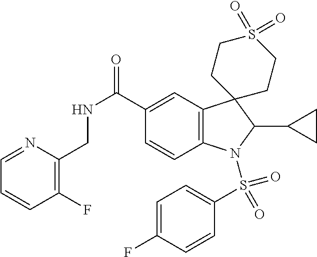 [SMILES: O=C(NCc1ncccc1F)c1ccc2c(c1)C1(CCS(=O)(=O)CC1)C(C1CC1)N2S(=O)(=O)c1ccc(F)cc1] | 2-cyclopropyl-1-[(4- fluorophenyl) sulfonyl]-N-[(3- fluoropyridin-2- yl)methyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.23-0.28 (m, 1H), 0.36-0.64 (m, 3H), 0.81-0.87 (m, 1H), 0.96-1.03 (m, 1H), 1.47 (dt, 1H), 2.55-2.63 (m, 3H), 3.19-3.22 (m, 2H), 3.62 (dt, 1H), 4.35 (d, 1H), 4.62-4.63 (m, 2H), 7.36-7.44 (m, 3H), 7.58 (d, 1H), 7.65-7.72 (m, 1H), 7.83 (mc, 1H), 7.86-7.91 (m, 3H), 8.35-8.38 (m, 1H), 9.01 (t, 1H). UPLC-MS (ESI+): [M + H]⁺ = 588. | from F.1 according to GP 9.1 |
| 53 | 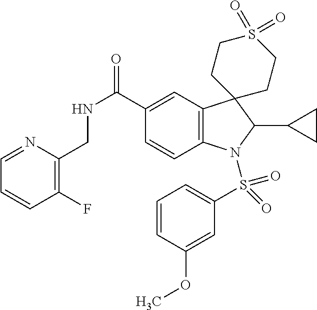 [SMILES: COc1cccc(S(=O)(=O)N2c3ccc(C(=O)NCc4ncccc4F)cc3C3(CCS(=O)(=O)CC3)C2C2CC2)c1] | 2-cyclopropyl-N-[(3- fluoropyridin-2- yl)methyl]-1-[(3- methoxyphenyl)sulfonyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.15-0.20 (m, 1H), 0.38-0.63 (m, 3H), 0.83-0.91 (m, 1H), 0.94-1.01 (m, 1H), 1.41 (dt, 1H), 2.42-2.62 (m, 3H), 3.19-3.23 (m, 2H), 3.64 (dt, 1H), 3.76 (s, 3H), 4.36 (d, 1H),4.61-4.63 (m, 2H), 7.19- 7.23 (m, 1H), 7.30-7.33 (m, 2H), 7.36-7.48 (m, 2H), 7.59 (d, 1H), 7.65-7.72 (m, 1H), 7.81 (d, 1H), 7.87 (dd, 1H), 8.36-8.38 (m, 1H), 9.00 (t, 1H). UPLC-MS (ESI+): [M + H]⁺ = 600. | from F.4 according to GP 9.1 |
| 54 | 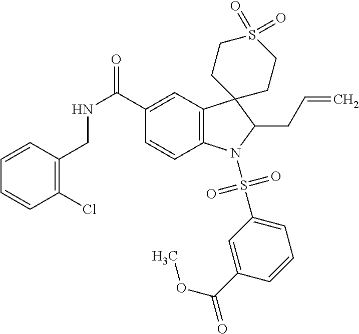 [SMILES: C=CCC1N(S(=O)(=O)c2cccc(C(=O)OC)c2)c2ccc(C(=O)NCc3ccccc3Cl)cc2C12CCS(=O)(=O)CC2] | methyl 3-({5-[N-(2- chlorobenzyl) carbamoyl]- 1′,1′-dioxido- 2-(prop-2-en-1-yl)- 1,2,2′,3′,5′,6′- hexahydrospiro [indole-3,4′-thiopyran]- 1-yl}sulfonyl) benzoate | ¹H-NMR (400 MHz, CDCl₃): Shift [ppm] = 1.32 (m, 1H), 2.00 (m, 1H), 2.15 (m, 1H), 2.68 (m, 1H), 2.83 (m, 1H), 3.02 (m, 2H), 3.15 (m, 1H), 3.98 (s, 1H), 4.73 (d, 2H), 4.80 (d, 1H), 5.35 (d, 1H), 5.52-5.75 (m, 2H), 6.51 (m, 1H), 7.40 (m, 1H), 7.49 (m, 1H), 7.58 (m, 3H), 7.68 (dbr, 1H), 8.04 (d, 1H), 8.25 (d, 1H), 8.54 (br. s., 1H). UPLC-MS (ESI+): [M + H]⁺ = 643/645 (Cl isotope pattern).. | prepared by carbonylation of intermediate D.3 according to GP 10 |
| 55 | 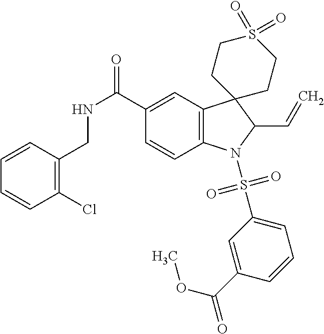 | methyl 3-({5-[N-(2- chlorobenzyl) carbamoyl]- 1′,1′-dioxido- 2-vinyl-1,2,2′,3′,5′,6′- hexahydrospiro [indole-3,4′-thiopyran]- 1-yl}sulfonyl) benzoate | ¹H-NMR (300 MHz, CDCl₃): Shift [ppm] = 0.69 (dbr, 1H), 0.88 (m, 2H), 2.32 (m, 2H), 2.52-2.72 (m, 3H), 2.95-3.20 (m, 3H), 3.97 (s, 3H), 4.46 (m, 1H), 4.72 (d, 2H), 5.00-5.13 (m, 2H), 5.70 (m, 1H), 6.53 (m, 1H), 7.39 (m, 1H), 7.48 (m, 1H), 7.56 (m, 2H), 7.68 (m, 2H), 7.97 (d, 1H), 8.24 (d, 1H), 8.50 (s, 1H). UPLC-MS (ESI+): [M + H]⁺ = 644. | prepared by carbonylation of intermediate D.4 according to GP 10 |
| 56 | 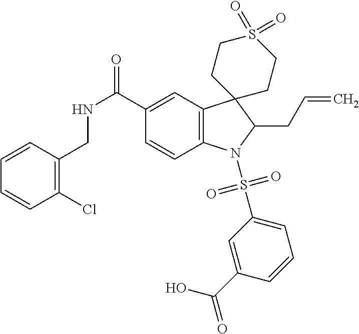 [SMILES: C=CCC1N(S(=O)(=O)c2cccc(C(=O)O)c2)c2ccc(C(=O)NCc3ccccc3Cl)cc2C12CCS(=O)(=O)CC2] | 3-({5-[(2- chlorobenzyl) carbamoyl]- 1′,1′-dioxido- 2-(prop-2-en-1-yl)- 2′,3′,5′,6′- tetrahydrospiro[indole- 3,4′-thiopyran]- 1(2H)-yl}sulfonyl) benzoic acid | ¹H-NMR (400 MHz, CDCl3): Shift [ppm] = 0.60 (dbr, 1H), 1.71 (tbr, 1H), 3.10-3.23 (m, 3H), 4.10 (t, 1H), 4.23 (d, 2H), 5.00-5.12 (m, 2H) 5.73 (m, 1H), 6.65 (t, 1H), 7.40 (m, 1H), 7.48 (m, 1H), 7.57 (t, 1H), 7.69 (m, 3H), 7.98 (d, 1H), 8.27 (d, 1H), 8.58 (s, 1H). UPLC-MS (ESI+): [M + H]⁺ = 629/631 (Cl isotope pattern).. | prepared by saponification of Ex. 54 according to GP 7 |
| 57 | 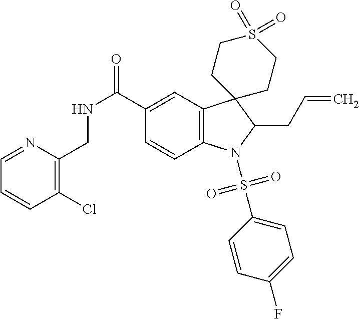 [SMILES: C=CCC1N(S(=O)(=O)c2ccc(F)cc2)c2ccc(C(=O)NCc3ncccc3Cl)cc2C12CCS(=O)(=O)CC2] | N-[(3-chloropyridin- 2-yl)methyl]-1-[(4- fluorophenyl)sulfonyl]- 2-(prop-2-en-1- yl)-1,2,2′,3′,5′,6′- hexahydrospiro [indole-3,4′-thiopyran]- 5-carboxamide 1′,1′-dioxide | ¹H-NMR (300 MHz, CDCl3): Shift [ppm] = 0.81 (dbr, 1H), 1.73 (tbr, 1H), 2.38 (m, 2H), 2.60-2.80 (m, 3H), 2.88-3.28 (m, 3H), 4.38 (t, 1H), 4.84 (d, 2H), 5.00- 5.15 (m, 2H), 5.72 (m, 1H), 7.16 (t, 2H), 7.29 (m, 1H), 7.18-7.80 (m, 3H), 7.81-7.95 (m, 4H), 8.02 (s, 1H). UPLC-MS (ESI+): [M + H]+ = 604/606 (Cl isotope pattern). | from F.11 and 1-(3- chloropyridin- 2-yl) methanamine according to GP 9.1 |
| 58 | 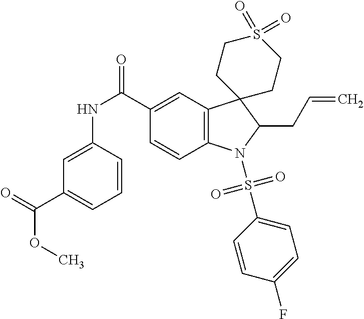 [SMILES: C=CCC1N(S(=O)(=O)c2ccc(F)cc2)c2ccc(C(=O)Nc3cccc(C(=O)OC)c3)cc2C12CCS(=O)(=O)CC2] | methyl 3-[({1-[(4- fluorophenyl)sulfonyl]- 1′,1′-dioxido-2- (prop-2-en-1-yl)- 1,2,2′,3′,5′,6′- hexahydrospiro [indole-3,4′- thiopyran]-5- yl}carbonyl)amino] benzoate | ¹H-NMR (300 MHz, CDCl3): Shift [ppm] = 0.80 (dbr, 1H), 1.77 (tbr, 1H), 2.40 m (2H), 2.60-2.80 (m, 3H), 2.92-3.25 (m, 3H), 3.94 (s, 3H), 4.40 (t, 1H), 5.00-5.15 (m, 2H), 5.70 (m, 1H), 7.16 (d, 2H), 7.48 (t, 1H), 7.73 (m, 2H), 7.80- 8.00 (m, 5H), 8.05-8.18 (m, 2H). UPLC-MS (ESI+): [M + H]+ = 613. | from F.11 and methyl 3-amino- benzoate according to GP 9.1 |
| 59 | 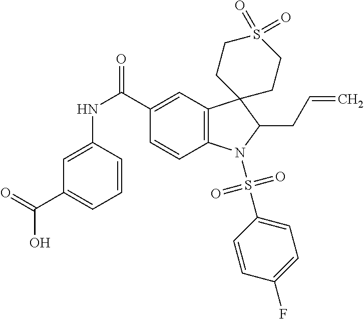 [SMILES: C=CCC1N(S(=O)(=O)c2ccc(F)cc2)c2ccc(C(=O)Nc3cccc(C(=O)O)c3)cc2C12CCS(=O)(=O)CC2] | 3-[({1-[(4- fluorophenyl)sulfonyl]- 1′,1′-dioxido-2- (prop-2-en-1-yl)- 1,2,2′,3′,5′,6′- hexahydrospiro [indole-3,4′- thiopyran]-5- yl}carbonyl)amino] benzoic acid | ¹H-NMR (300 MHz, MeOD): Shift [ppm] = 0.60 (dbr, 1H), 1.70 (tbr, 1H), 2.45 (m, 2H), 2.55- 2.80 (m, 3H), 3.13 (m, 1H), 3.40 (m, 1H), 4.72 (t, 1H), 4.95 (d, 1H), 5.08 (d, 1H), 5.75 (m, 1H), 7.29 (t, 2H), 7.48 t (1H), 7.75 (d, 1H), 7.80 (m, 2H), 7.90-8.08 (m, 4H), 8.35 (s, 1H). UPLC-MS (ESI+): [M + H]+ = 599. | prepared by saponification of Ex. 58 according to GP 7 |
| 60 | 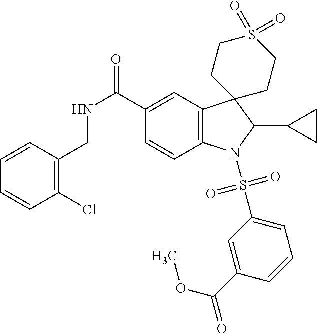 [SMILES: COC(=O)c1cccc(S(=O)(=O)N2c3ccc(C(=O)NCc4ccccc4Cl)cc3C3(CCS(=O)(=O)CC3)C2C2CC2)c1] | methyl 3-({5-[(2- chlorobenzyl) carbamoyl]-2- cyclopropyl-1′,1′- dioxido-2′,3′,5′,6′- tetrahydrospiro[indole- 3,4′-thiopyran]- 1(2H)-yl} sulfonyl)benzoate | ¹H-NMR (300 MHz, DMSO-d6): Shift [ppm] = 0.26-0.37 (m, 1H), 0.37-0.47 (m, 1H), 0.48-0.65 (m, 2H), 0.85-1.07 (m, 2H), 1.47 (dt, 1H), 3.65 (dt, 1H), 3.87 (s, 3H), 4.45 (d, 1H), 4.52 (d, 2H), 7.25-7.49 (m, 4H), 7.60 (d, 1H), 7.73 (tr, 1H), 7.84 (s, 1H), 7.92 (dd, 1H), 8.10 (d, 1H), 8.19 (d, 1H), 8.31 (s, 1H), 9.02 (t, 1H). UPLC-MS (ESI+): [M + H]+ = 643. | from F.12 and 1-(2- chlorophenyl) methanamine according to GP 9.1 |
| 60.1 | Enantiomer 1 of Ex. 60 | | Rt = 10.31 min | HPLC |
| 60.2 | Enantiomer 2 of Ex. 60 | | Rt = 15.93 min | method |
| | | | | A with |
| | | | | column: |
| | | | | Chiralpak IA |
| | | | | 5 μm |
| | | | | 150 × 4.6 |
| | | | | mm; Solvent |
| | | | | Hexan/ |
| | | | | Ethanol 70:30 |
| | | | | (v/v); |
| | | | | Detection: |
| | | | | DAD 280 nm |
| 61.1 | 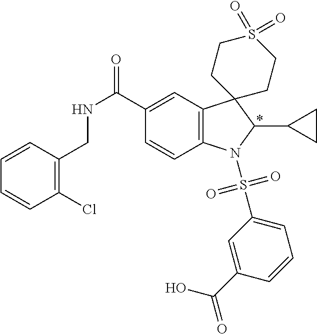 [SMILES: O=C(O)c1cccc(S(=O)(=O)N2c3ccc(C(=O)NCc4ccccc4Cl)cc3C3(CCS(=O)(=O)CC3)C2C2CC2)c1] | 3-({5-[(2- chlorobenzyl) carbamoyl]-2- cyclopropyl-1′,1′- dioxido-2′,3′,5′,6′- tetrahydrospiro[indole- 3,4′-thiopyran]- 1(2H)-yl} sulfonyl)benzoic acid | ¹H-NMR (400 MHz, DMSO-d6): Shift [ppm] = 0.23-0.32 (m, 1H), 0.37-0.46 (m, 1H), 0.48-0.62 (m, 2H), 0.88-1.03 (m, 2H), 1.46 (dt, 1H), 3.66 (dt, 1H), 4.44 (d, 1H), 4.52 (m, 2H), 7.25-7.48 (m, 4H), 7.60 (d, 1H), 7.69 (t, 1H), 7.84 (m, 1H), 7.92 (dd, 1H), 8.05 (d, 1H), 8.17 (d, 1H), 8.31 (s, 1H), 9.02 (t, 1H), 13.57 (br. s., 1H). UPLC-MS (ESI+): [M + H]+ = 629. | prepared by saponification of Ex. 60.1 according to GP 7 |
| 361.2 | Enantiomer 2 of Ex. 61.1 | 3-({5-[(2- | | prepared by |
| | | chlorobenzyl) | | saponification |
| | | carbamoyl]-2- | | of Ex. 60.2 |
| | | cyclopropyl-1′,1′- | | according to |
| | | dioxido-2′,3′,5′,6′- | | GP 7 |
| | | tetrahydrospiro[indole- | | |
| | | 3,4′-thiopyran]- | | |
| | | 1(2H)-yl} | | |
| | | sulfonyl)benzoic acid | | |
| 62 | 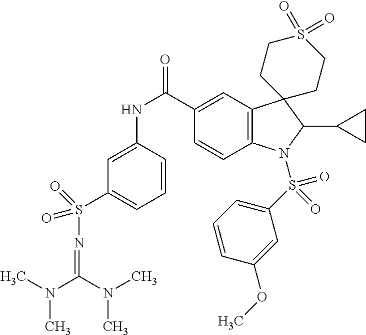 [SMILES: COc1cccc(S(=O)(=O)N2c3ccc(C(=O)Nc4cccc(S(=O)(=O)N=C(N(C)C)N(C)C)c4)cc3C3(CCS(=O)(=O)CC3)C2C2CC2)c1] | N-(3- {[bis(dimethylamino) methylidene]sulfamoyl} phenyl)-2- cyclopropyl-1-[(3- methoxyphenyl) sulfonyl]-1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.16-0.26 (m, 1H), 0.37-0.48 (m, 1H), 0.48-0.66 (m, 2H), 0.83-0.93 (m, 1H), 0.94-1.06 (m, 1H), 1.48 (dt, 1H), 2.85 (s, 12H), 3.67 (dt, 1H), 3.77 (s, 3H), 4.40 (d, 1H), 7.20- 7.26 (m, 1H), 7.30-7.38 (m, 2H), 7.42-7.51 (m, 3H), 7.65 (d, 1H), 7.84-7.92 (m, 2H), 7.94- 8.00 (m, 1H), 8.21 (s, 1H), 10.35 (s, 1H). UPLC-MS (ESI+): [M + H]⁺ = 744. | from F.4 and 3-amino- benzene- sulfonamide (CAS No. [98-18-0]) according to GP 9.1 |
| 62.1 | Enantiomer 1 of Ex. 62 | | Rt = 9.20 min | HPLC |
| 62.2 | Enantiomer 2 of Ex. 62 | | Rt = 11.75 min | method |
| | | | | A with |
| | | | | column: |
| | | | | Chiralpak IC |
| | | | | 5 μm |
| | | | | 150 × 4.6 |
| | | | | mm; Solvent |
| | | | | Methanol; |
| | | | | Detection: |
| | | | | DAD 280 nm |
| 63 | 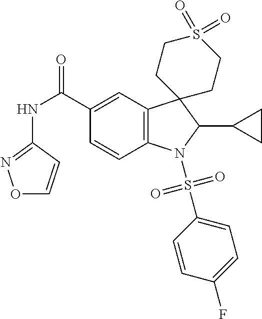 [SMILES: O=C(Nc1ccon1)c1ccc2c(c1)C1(CCS(=O)(=O)CC1)C(C1CC1)N2S(=O)(=O)c1ccc(F)cc1] | 2-cyclopropyl-1-[(4- fluorophenyl)sulfonyl]- N-(1,2-oxazol-3- yl)-1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.30 (d, 1H), 0.35- 0.43 (m, 1H), 0.45-0.53 (m, 1H), 0.53-0.62 (m, 1H), 0.77- 0.86 (m, 1H), 0.92-1.04 (m, 1H), 1.50 (dt, 1H), 3.59 (dt, 1H), 4.34 (d, 1H), 7.00 (m, 1H), 7.34- 7.41 (m, 2H), 7.59 (d, 1H), 7.84- 8.04 (m, 4H), 8.80 (m, 1H), 11.37 (s, 1H). UPLC-MS (ESI+): [M + H]+ = 546. | from F.1 according to GP 9.2 |
| 63.1 | Enantiomer 1 of Ex. 63 | | Rt = 2.48 min | HPLC |
| 63.2 | Enantiomer 2 of Ex. 63 | | Rt = 3.30 min | method |
| | | | | A with |
| | | | | column: |
| | | | | Chiralpak IC |
| | | | | 5 μm |
| | | | | 100 × 4.6 |
| | | | | mm; Solvent |
| | | | | Ethanol/ |
| | | | | Methanol |
| | | | | 50:50 (v/v); |
| | | | | Detection: |
| | | | | DAD 280 nm |
| 64 | 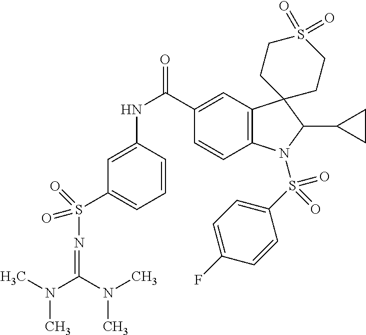 [SMILES: CN(C)C(=NS(=O)(=O)c1cccc(NC(=O)c2ccc3c(c2)C2(CCS(=O)(=O)CC2)C(C2CC2)N3S(=O)(=O)c2ccc(F)cc2)c1)N(C)C] | N-(3- {[bis(dimethylamino) methylidene]sulfamoyl} phenyl)-2- cyclopropyl-1-[(4- fluorophenyl)sulfonyl]- 1,2,2′,3′,5′,6′- hexahydrospiro [indole-3,4′-thiopyran]- 5-carboxamide 1′,1′-dioxide | 1H-NMR (300 MHz, DMSO-d6): Shift [ppm] = 0.24-0.64 (m, 4H), 0.81-0.91 (m, 1H), 0.94-1.07 (m, 1H), 1.53 (dt, 1H), 2.85 (s, 12H), 3.66 (dt, 1H), 4.40 (d, 1H), 7.37-7.50 (m, 4H), 7.64 (d, 1H), 7.84-8.01 (m, 5H), 8.21 (s, 1H), 10.35 (s, 1H). UPLC-MS (ESI+): [M + H]+ = 732. | from F.1 and 3-amino- benzene- sulfonannide (CAS No. [98-18-0]) according to GP 9.1 |
| 64.1 | Enantiomer 1 of Ex. 64 | | Rt = 9.43 min | HPLC |
| 64.2 | Enantiomer 2 of Ex. 64 | | Rt = 10.86 min | method |
| | | | | A with |
| | | | | column: |
| | | | | Chiralpak IC |
| | | | | 5 μm |
| | | | | 150 × 4.6 |
| | | | | mm; Solvent |
| | | | | Methanol; |
| | | | | Detection: |
| | | | | DAD 280 nm |
| 65 | 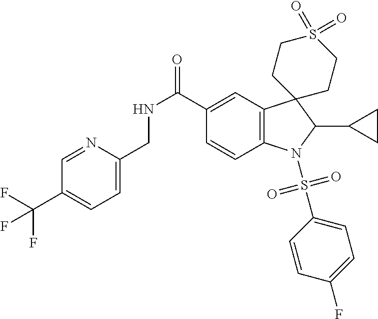 [SMILES: O=C(NCc1ccc(C(F)(F)F)cn1)c1ccc2c(c1)C1(CCS(=O)(=O)CC1)C(C1CC1)N2S(=O)(=O)c1ccc(F)cc1] | 2-cyclopropyl-1-[(4- fluorophenyl) sulfonyl]-N-{[5- (trifluoromethyl) pyridin-2-yl]methyl}- 1,2,2′,3′,5′,6′- hexahydrospiro [indole-3,4′-thiopyran]- 5-carboxamide 1′,1′-dioxide | ¹H-NMR (400 MHz, DMSO-d6): Shift [ppm] = 0.24-0.34 (m, 1H), 0.36-0.46 (m, 1H), 0.46-0.55 (m, 1H), 0.55-0.60 (m, 1H), 0.81-0.89 (m, 1H), 0.94-1.05 (m, 1H), 1.48 (dt, 1H), 3.63 (dt, 1H), 4.37 (d, 1H), 4.63 (d, 2H), 7.37-7.45 (m, 2H), 7.53-7.64 (m, 2H), 7.83-7.94 (m, 4H), 8.16 (m, 1H) , 8.90 (s, 1H), 9.23 (m, 1H), 10.35 (s, 1H). UPLC-MS (ESI+): [M + H]+ = 636. | from F.1 and 1-[5- (trifluoro- methyl) pyridin-2-yl] methanamine hydrochloride (CAS No. [871826-12-9]) according to GP 9.1 |
| 66 | 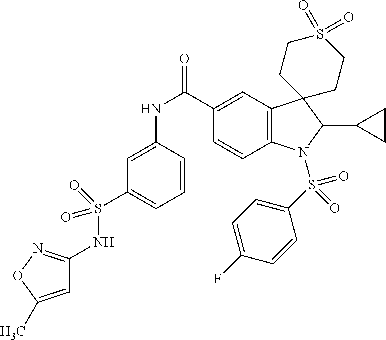 [SMILES: Cc1cc(NS(=O)(=O)c2cccc(NC(=O)c3ccc4c(c3)C3(CCS(=O)(=O)CC3)C(C3CC3)N4S(=O)(=O)c3ccc(F)cc3)c2)no1] | 2-cyclopropyl-1-[(4- fluorophenyl)sulfonyl]- N-{3-[(5-methyl- 1,2-oxazol-3-yl) sulfamoyl]phenyl}- 1,2,2′,3′,5′,6'- hexahydrospiro [indole-3,4′-thiopyran]- 5-carboxamide 1′,1′-dioxide | ¹H-NMR (400 MHz, DMSO-d6): Shift [ppm] = 0.28-0.37 (m, 1H), 0.38-0.47 (m, 1H), 0.47-0.56 (m, 1H), 0.57-0.67 (m, 1H), 0.82-0.91 (m, 1H), 0.96-1.06 (m, 1H), 1.54 (dt, 1H), 2.30 (s, 3H), 3.66 (dt, 1H), 4.40 (d, 1H), 6.13 (s, 1H), 7.38-7.47 (m, 2H), 7.53-7.67 (m, 3H), 7.87-8.02 (m, 5H), 8.34 (s, 1H), 10.48 (s, 1H). UPLC-MS (ESI+): [M + H]+ = 715. | from F.1 and 3-amino-N- (5-methyl- 1,2-oxazol-3- yl)benzene- sulfon- amide according to GP 9.2 |
| 67 | 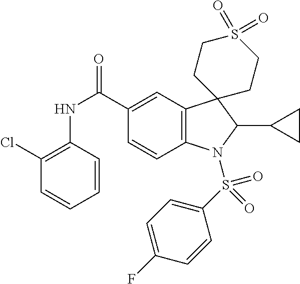 [SMILES: O=C(Nc1ccccc1Cl)c1ccc2c(c1)C1(CCS(=O)(=O)CC1)C(C1CC1)N2S(=O)(=O)c1ccc(F)cc1] | N-(2-chlorophenyl)- 2-cyclopropyl-1-[(4- fluorophenyl)sulfonyl]- 1,2,2′,3′,5′,6′- hexahydrospiro [indole-3,4′-thiopyran]- 5-carboxamide 1′,1′-dioxide | ¹H-NMR (300 MHz, DMSO-d6): Shift [ppm] = 0.26-0.37 (m, 1H), 0.38-0.49 (m, 1H), 0.49-0.57 (m, 1H), 0.57-0.67 (m, 1H), 0.81-0.92 (m, 1H), 0.96-1.09 (m, 1H), 1.52 (dt, 1H), 3.65 (dt, 1H), 4.39 (d, 1H), 7.26-7.68 (m, 7H), 7.87-8.01 (m, 4H), 10.08 (s, 1H). UPLC-MS (ESI+): [M + H]+ = 589/591 (Cl isotope pattern). | from F.1 and 2- chloroaniline according to GP 9.2 |
| 68 | 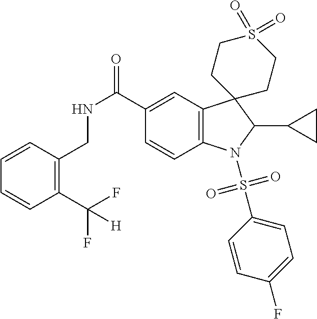 [SMILES: O=C(NCc1ccccc1C(F)F)c1ccc2c(c1)C1(CCS(=O)(=O)CC1)C(C1CC1)N2S(=O)(=O)c1ccc(F)cc1] | 2-cyclopropyl-N-[2- (difluoromethyl) benzyl]-1-[(4- fluorophenyl)sulfonyl]- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.24-0.33 (m, 1H), 0.37-0.46 (m, 1H), 0.46-0.55 (m, 1H), 0.55-0.65 (m, 1H), 0.81-0.90 (m, 1H), 0.94-1.05 (m, 1H), 1.48 (dt, 1H), 3.63 (dt, 1H), 4.36 (d, 1H), 4.61 (d, 2H), 7.34 (t, 1H), 7.37-7.61 (m, 7H), 7.81 (m, 1H), 7.86-7.93 (m, 3H), 9.06 (t, 1H). UPLC-MS (ESI+): [M + H]+ = 619. | from F.1 and 1-[2- (difluoro- methyl) phenyl] methanamine according to GP 9.1 |
| 68.1 | Enantiomer 1 of Ex 68 | | Rt = 1.24 min | HPLC |
| 68.2 | Enantiomer 2 of Ex. 68 | | Rt = 1.89 min | method C |
| | | | | with column: |
| | | | | Chiralpak IA |
| | | | | 3 μm |
| | | | | 100 × 4.6 |
| | | | | mm; Solvent |
| | | | | CO₂/Ethanol |
| | | | | +0.2% vol. |
| | | | | Et₂NH 70:30 |
| | | | | (v/v); |
| | | | | Detection: |
| | | | | DAD 280 nm |
| 69 | 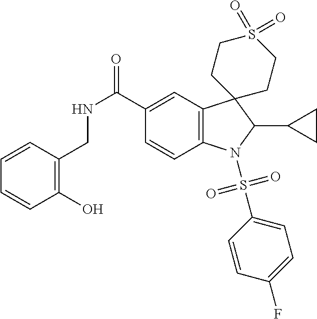 [SMILES: O=C(NCc1ccccc1O)c1ccc2c(c1)C1(CCS(=O)(=O)CC1)C(C1CC1)N2S(=O)(=O)c1ccc(F)cc1] | 2-cyclopropyl-1-[(4- fluorophenyl)sulfonyl]- N-(2-hydroxybenzyl)- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.21-0.33 (m, 1H), 0.37-0.46 (m, 1H), 0.46-0.55 (m, 1H), 0.54-0.65 (m, 1H), 0.79-0.90 (m, 1H), 0.92-1.06 (m, 1H), 1.48 (dt, 1H), 3.63 (dt, 1H), 4.35 (d, 1H), 4.39 (d, 2H), 6.70-6.83 (m, 2H), 7.02-7.15 (m, 2H), 7.35-7.45 (m, 2H), 7.58 d, 1H), 7.81-7.94 (m, 4H), 8.94 (t, 1H), 9.57 (s, IH). UPLC-MS (ESI+): [M + H]+ = 585. | from F.1 and 2-(amino- methyl) phenol according to GP 9.1 |
| 70 | 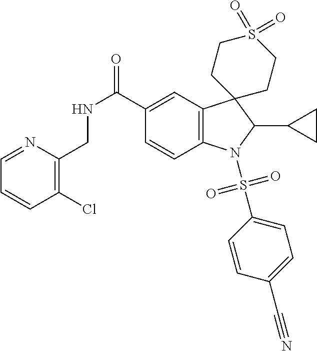 [SMILES: N#Cc1ccc(S(=O)(=O)N2c3ccc(C(=O)NCc4ncccc4Cl)cc3C3(CCS(=O)(=O)CC3)C2C2CC2)cc1] | N-[(3-chloropyridin- 2-yl)methyl]-1-[(4- cyanophenyl)sulfonyl]- 2-cyclopropyl- 1,2,2′,3′,5′,6′- hexahydrospiro [indole-3,4′-thiopyran]- 5-carboxamide 1′,1′-dioxide | ¹H-NMR (400 MHz, DMSO-d6): Shift [ppm] = 0.14-0.24 (m, 1H), 0.38-0.48 (m, 1H), 0.49-0.57 (m, 1H), 0.57-0.66 (m, 1H), 0.79-0.89 (m, 1H), 0.94-1.07 (m, 1H), 1.46 (dt, 1H), 3.61 (dt, 1H), 4.37 (d, 1H), 4.67 (d, 2H), 7.33-7.40 (m, 1H), 7.62 (d, 1H), 7.85-8.08 (m, 7H), 8.48 (m, 1H), 8.99 (m, 1H). UPLC-MS (ESI+): [M + H]+ = 611/613 (Cl isotope pattern). | from F.8 and 1-(3-chloro- pyridin- 2-yl) methanamine according to GP 9.1 |
| 70.1 | Enantiomer 1 of Ex. 70 | | Rt = 2.81 min | HPLC |
| 70.2 | Enantiomer 2 of Ex. 70 | | Rt = 3.45 min | method C |
| | | | | with column: |
| | | | | Chiralpak ID |
| | | | | 3 μm |
| | | | | 100 × 4.6 |
| | | | | mm; Solvent |
| | | | | CO2/ |
| | | | | Methanol + |
| | | | | 0.2% |
| | | | | vol. Et2NH |
| | | | | 60:40 (v/v); |
| | | | | Detection: |
| | | | | DAD 254 nm |
| 71 | 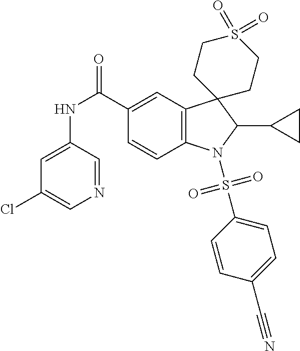 [SMILES: N#Cc1ccc(S(=O)(=O)N2c3ccc(C(=O)Nc4cncc(Cl)c4)cc3C3(CCS(=O)(=O)CC3)C2C2CC2)cc1] | N-(5-chloropyridin- 3-yl)-1-[(4- cyanophenyl)sulfonyl]- 2-cyclopropyl- 1,2,2′,3′,5′,6′- hexahydrospiro [indole-3,4′-thiopyran]- 5-carboxamide 1′,1′-dioxide | ¹H-NMR (300 MHz, DMSO-d6): Shift [ppm] = 0.16-0.29 (m, 1H), 0.38-1.09 (m, 5H), 1.51 (dt, 1H), 3.64 (dt, 1H), 4.42 (d, 1H), 7.68 (d, 1H), 7.91 (m, 1H), 7.95- 8.09 (m, 5H), 8.30-8.39 (m, 2H), 8.82 (m, 2H), 10.53 (s, 1H). UPLC-MS (ESI+): [M + H]+ = 597/599 (Cl isotope pattern). | from F.8 and 5- chloropyridin- 3-amine according to GP 9.2 |
| 71.1 | Enantiomer 1 of Ex. 71 | | Rt = 5.57 min | HPLC |
| 71.2 | Enantiomer 2 of Ex. 71 | | Rt = 6.96 min | method A |
| | | | | with column: |
| | | | | Chiralpak IC |
| | | | | 3 μm |
| | | | | 100 × 4.6 |
| | | | | mm; Solvent |
| | | | | Ethanol/ |
| | | | | Methanol |
| | | | | 50:50 (v/v); |
| | | | | Detection: |
| | | | | DAD 280 nm |
| 72 | 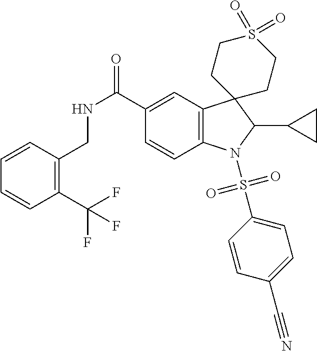 [SMILES: N#Cc1ccc(S(=O)(=O)N2c3ccc(C(=O)NCc4ccccc4C(F)(F)F)cc3C3(CCS(=O)(=O)CC3)C2C2CC2)cc1] | 1-[(4-cyanophenyl) sulfonyl]- 2-cyclopropyl-N- [2-(trifluoromethyl) benzyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.14-0.25 (m, 1H), 0.38-0.49 (m, 1H), 0.50-0.67 (m, 2H), 0.78-0.91 (m, 1H), 0.95-1.07 (m, 1H), 1.47 (dt, 1H), 3.62 (dt, 1H), 4.37 (d, 1H), 4.65 (d, 2H), 7.43-8.08 (m, 11H), 9.13 (m, 1H). UPLC-MS (ESI+): [M + H]+ = 644. | from F.8 and 1-[2- (trifluoro- methyl) phenyl] methanamine according to GP 9.1 |
| 72.1 | Enantiomer 1 of Ex. 72 | | Rt = 4.83 min | HPLC |
| 72.2 | Enantiomer 2 of Ex. 72 | | Rt = 7.32 min | method A |
| | | | | with column: |
| | | | | Chiralpak IA |
| | | | | 3 μm |
| | | | | 100 × 4.6 |
| | | | | mm; Solvent |
| | | | | Hexane/ |
| | | | | Ethanol |
| | | | | 70:30 (v/v); |
| | | | | Detection: |
| | | | | DAD 280 nm |
| 73 | 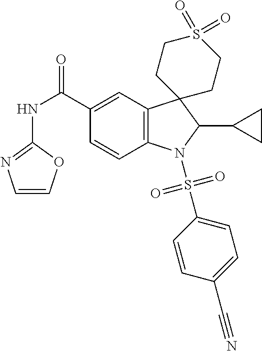 [SMILES: N#Cc1ccc(S(=O)(=O)N2c3ccc(C(=O)Nc4ncco4)cc3C3(CCS(=O)(=O)CC3)C2C2CC2)cc1] | 1-[(4- cyanophenyl)sulfonyl]- 2-cyclopropyl-N- (1,3-oxazol-2-yl)- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.16-0.33 (m, 1H), 0.36-0.49 (m, 1H), 0.50-0.67 (m, 2H), 0.77-0.91 (m, 1H), 0.92-1.08 (m, 1H), 1.50 (dt, 1H), 3.61 (dt, 1H), 4.37 (d, 1H), 7.57-8.11 (m, 9H). UPLC-MS (ESI+): [M + H]+ = 553. | from F.8 and 1,3-oxazol-2- amine according to GP 9.2 |
| 74 | 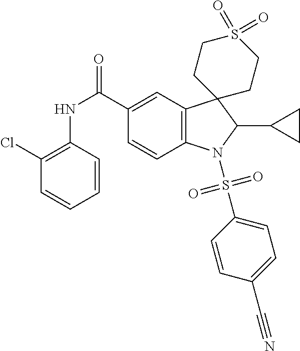 [SMILES: N#Cc1ccc(S(=O)(=O)N2c3ccc(C(=O)Nc4ccccc4Cl)cc3C3(CCS(=O)(=O)CC3)C2C2CC2)cc1] | N-(2-chlorophenyl)- 1-[(4-cyanophenyl) sulfonyl]- 2-cyclopropyl- 1,2,2′,3′,5′,6′- hexahydrospiro[indole- 3,4′-thiopyran]- 5-carboxamide 1′,1′-dioxide | UPLC-MS (ESI+): [M + H]+ = 596/598 (Cl isotope pattern). | from F.8 and 2-chloroaniline according to GP 9.2 |
| 75 | 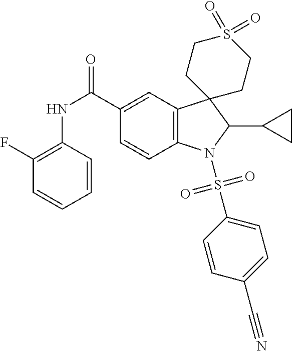 [SMILES: N#Cc1ccc(S(=O)(=O)N2c3ccc(C(=O)Nc4ccccc4F)cc3C3(CCS(=O)(=O)CC3)C2C2CC2)cc1] | 1-[(4- cyanophenyl)sulfonyl]- 2-cyclopropyl-N- (2-fluorophenyl)- 1,2,2′,3′,5',6′- hexahydrospiro[indole- 3,4′-thiopyran]- 5-carboxamide 1′,1′-dioxide | ¹H-NMR (300 MHz, DMSO-d6): Shift [ppm] = 0.14 (d, 1H), 0.35- 0.46 (m, 1H), 0.47-0.64 (m, 2H), 0.75-0.87 (m, 2H), 0.93- 1.07 (m, 2H), 1.40-1.51 (m, 2H), 2.58-2.72 (m, 1H), 3.09- 3.24 (m, 1H), 3.52-3.65 (m, 1H), 4.36 (d, 1H), 7.61 (d, 1H), 7.15-7.31 (m, 3H), 7.46-7.53 (m, 1H), 7.60-7.86 (m, 1H), 7.89-8.06 (m, 6H), 10.09 (s, 1H). UPLC-MS (ESI+): [M + H]+ = 580. | from F.8 and 2- fluoroaniline according to GP 9.2 |
| 76 | 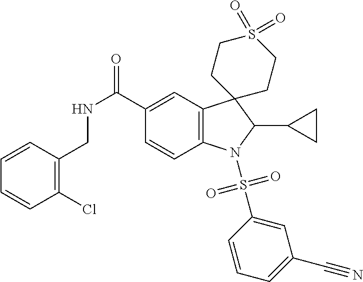 [SMILES: N#Cc1cccc(S(=O)(=O)N2c3ccc(C(=O)NCc4ccccc4Cl)cc3C3(CCS(=O)(=O)CC3)C2C2CC2)c1] | N-(2-chlorobenzyl)- 1-[(3-cyanophenyl) sulfonyl]- 2-cyclopropyl- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.05-0.18 (m, 1H), 0.38-0.50 (m, 1H), 0.55-0.68 (m, 2H), 0.77-0.89 (m, 1H), 0.93-1.07 (m, 1H), 1.47 (dt, 1H), 3.70 (dt, 1H), 4.35 (d, 1H), 4.53 (d, 2H), 7.26-8.15 (m, 10H), 8.43 (m, 1H), 9.06 (m, 1H). UPLC-MS (ESI+): [M + H]+ = 610/612 (Cl isotope pattern). | from F.9 and 1-(2- chlorophenyl) methanamine according to GP 9.1 |
| 76.1 | Enantiomer 1 of Ex. 76 | | Rt = 7.3 min | HPLC |
| 76.2 | Enantiomer 2 of Ex. 76 | | Rt = 8.8 min | method A |
| | | | | with column: |
| | | | | Chiralpak IB |
| | | | | 3 μm |
| | | | | 100 × 4.6 |
| | | | | mm; Solvent |
| | | | | Hexane/ |
| | | | | Ethanol 70:30 |
| | | | | (v/v); |
| | | | | Detection: |
| | | | | DAD 280 nm |
| 77 | 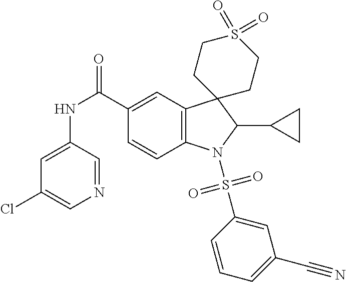 [SMILES: N#Cc1cccc(S(=O)(=O)N2c3ccc(C(=O)Nc4cncc(Cl)c4)cc3C3(CCS(=O)(=O)CC3)C2C2CC2)c1] | N-(5-chloropyridin- 3-yl)-1-[(3- cyanophenyl)sulfonyl]- 2-cyclopropyl- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.11-0.23 (m, 1H), 0.38-0.52 (m, 1H), 0.54-0.70 (m, 2H), 0.77-0.90 (m, 1H), 0.95-1.07 (m, 1H), 1.52 (dt, 1H), 3.72 (dt, 1H), 4.40 (d, 1H), 7.66-8.17 (m, 6H), 8.29-8.46 (m, 3H), 8.82 (d, 1H), 10.53 (s, 1H). UPLC-MS (ESI+): [M + H]+ = 597/599 (Cl isotope pattern). | from F.9 and 5-chloro- pyridin- 3-amine according to GP 9.2 |
| 77.1 | Enantiomer 1 of Ex. 77 | | Rt = 5.4 min | HPLC |
| 77.2 | Enantiomer 2 of Ex. 77 | | Rt = 8.2 min | method A |
| | | | | with column: |
| | | | | Chiralpak IA |
| | | | | 3 μm |
| | | | | 100 × 4.6 |
| | | | | mm; Solvent |
| | | | | Hexane/2- |
| | | | | Propanol |
| | | | | 70:30 |
| | | | | (v/v) +0.1% |
| | | | | Et₂NH; |
| | | | | Detection: |
| | | | | DAD 254 nm |
| 78 | 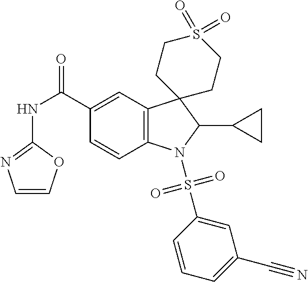 [SMILES: N#Cc1cccc(S(=O)(=O)N2c3ccc(C(=O)Nc4ncco4)cc3C3(CCS(=O)(=O)CC3)C2C2CC2)c1] | 1-[(3-cyanophenyl) sulfonyl]- 2-cyclopropyl-N- (1,3-oxazol-2-yl)- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.14-0.28 (m, 1H), 0.38-0.51 (m, 1H), 0.54-0.69 (m, 2H), 0.76-0.89 (m, 1H), 0.91-1.08 (m, 1H), 1.50 (dt, 1H), 3.68 (dt, 1H), 4.36 (d, 1H), 7.10-8.47 (m, 9H). UPLC-MS (ESI+): [M + H]+ = 553. | from F.9 and 1,3-oxazol-2- amine according to GP 9.2 |
| 79 | 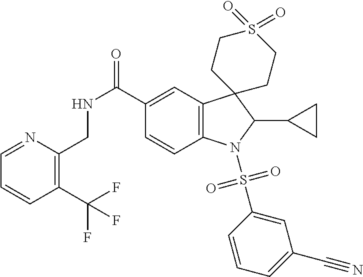 [SMILES: N#Cc1cccc(S(=O)(=O)N2c3ccc(C(=O)NCc4ncccc4C(F)(F)F)cc3C3(CCS(=O)(=O)CC3)C2C2CC2)c1] | 1-[(3- cyanophenyl)sulfonyl]- 2-cyclopropyl-N- {[3-(trifluoromethyl) pyridin- 2-yl]methyl}- 1,2,2′,3′,5′,6′- hexahydrospiro[indole- 3,4′-thiopyran]- 5-carboxamide 1′,1′-dioxide | ¹H-NMR (500 MHz, DMSO-d6): Shift [ppm] = 0.09-0.18 (m, 1H), 0.40-0.48 (m, 1H), 0.56-0.66 (m, 2H), 0.79-0.86 (m, 1H), 0.96-1.05 (m, 1H), 1.46 (dt, 1H), 3.70 (dt, 1H), 4.35 (d, 1H), 4.73 (m, 2H), 7.51-7.56 (m, 1H), 7.64 (d, 1H), 7.75 (t, 1H), 7.84-7.93 (m, 2H), 8.02 (m, 1H), 8.13 (m, 1H), 8.18 (m, 1H), 8.43 (m, 1H), 8.79 (d, 1H), 9.06 (m, 1H). UPLC-MS (ESI+): [M + H]+ = 645. | from F.9 and 1-[3- (trifluoro- methyl) pyridin-2-yl] methanamine according to GP 9.1 |
| 79.1 | Enantiomer 1 of Ex. 79 | | Rt = 2.82 min | HPLC |
| 79.2 | Enantiomer 2 of Ex. 79 | | Rt = 3.23 min | method A |
| | | | | with column: |
| | | | | Chiralpak IC |
| | | | | 3 μm |
| | | | | 100 × 4.6 |
| | | | | mm; Solvent |
| | | | | Ethanol/ |
| | | | | Methanol |
| | | | | 50:50 (v/v); |
| | | | | Detection: |
| | | | | DAD 280 nm |
| 80 | 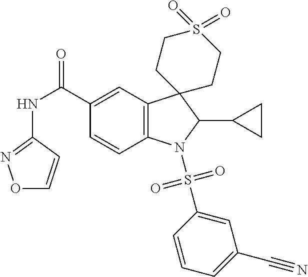 [SMILES: N#Cc1cccc(S(=O)(=O)N2c3ccc(C(=O)Nc4ccon4)cc3C3(CCS(=O)(=O)CC3)C2C2CC2)c1] | 1-[(3- cyanophenyl)sulfonyl]- 2-cyclopropyl-N- (1,2-oxazol-3-yl)- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.15-0.24 (m, 1H), 0.39-0.49 (m, 1H), 0.57-0.68 (m, 2H), 0.79-0.87 (m, 1H), 0.95-1.06 (m, 1H), 1.51 (dt, 1H), 3.68 (dt, 1H), 4.36 (d, 1H), 7.04 (m, 1H), 7.68 (d, 1H), 7.76 (t, 1H), 7.98-8.08 (m, 3H), 8.13 (d, 1H), 8.44 (s, 1H), 8.84 (m, 1H), 11.42 (s, 1H). UPLC-MS (ESI+): [M + H]+ = 553. | from F.9 and 1,2-oxazol-3- amine according to GP 9.2 |
| 80.1 | Enantiomer 1 of Ex. 80 | | Rt = 2.87 min | HPLC |
| 80.2 | Enantiomer 2 of Ex. 80 | | Rt = 4.06 min | method A |
| | | | | with column: |
| | | | | Chiralpak IC |
| | | | | 3 μm |
| | | | | 100 × 4.6 |
| | | | | mm; Solvent |
| | | | | Ethanol/ |
| | | | | Methanol |
| | | | | 50:50 (v/v); |
| | | | | Detection: |
| | | | | DAD 280 nm |
| 81 | 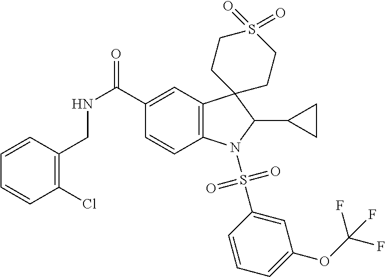 [SMILES: O=C(NCc1ccccc1Cl)c1ccc2c(c1)C1(CCS(=O)(=O)CC1)C(C1CC1)N2S(=O)(=O)c1cccc(OC(F)(F)F)c1] | N-(2-chlorobenzyl)- 2-cyclopropyl-1-{[3- (trifluoromethoxy) phenyl]sulfonyl}- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.10-0.24 (m, 1H), 0.32-0.44 (m, 1H), 0.45-0.64 (m, 2H), 0.75-0.87 (m, 1H), 0.90-1.03 (m, 1H), 1.44 (dt, 1H), 3.62 (dt, 1H), 4.25 (d, 1H), 4.49 (d, 2H), 7.18-7.94 (m, 11H), 9.02 (t, 1H). UPLC-MS (ESI+): [M + H]+ = 669/671 (Cl isotope pattern). | prepared by carbonylation of intermediate D.8 with 1-(2- chlorophenyl) methanamine according to GP 10 |
| 82 | 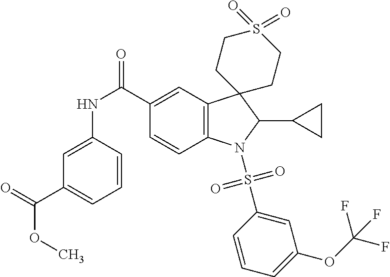 [SMILES: COC(=O)c1cccc(NC(=O)c2ccc3c(c2)C2(CCS(=O)(=O)CC2)C(C2CC2)N3S(=O)(=O)c2cccc(OC(F)(F)F)c2)c1] | methyl 3-{[(2- cyclopropyl-1′,1′- dioxido-1-{[3- (trifluoromethoxy) phenyl]sulfonyl}- 1,2,2′,3′,5′,6′- hexahydrospiro[indole- 3,4′-thiopyran]- yl)carbonyl]amino} benzoate | ¹H-NMR (400 MHz, DMSO-d6): Shift [ppm] = 0.22-0.33 (m, 1H), 0.38-0.48 (m, 1H), 0.50-0.68 (m, 2H), 0.82-0.91 (m, 1H), 0.97-1.07 (m, 1H), 1.54 (dt, 1H), 3.68 (dt, 1H), 3.87 (s, 3H), 4.41 (d, 1H), 7.48-8.10 (m, 10H), 8.37 (m, 1H), 10.36 (s, 1H). UPLC-MS (ESI+): [M + H]+ = 679. | from F.5 and methyl 3- aminobenzoate according to GP 9.1 |
| 83 | 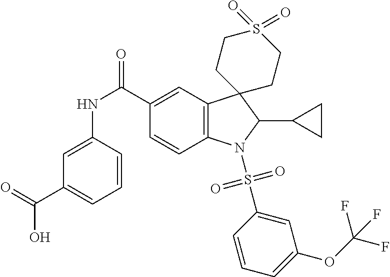 [SMILES: O=C(O)c1cccc(NC(=O)c2ccc3c(c2)C2(CCS(=O)(=O)CC2)C(C2CC2)N3S(=O)(=O)c2cccc(OC(F)(F)F)c2)c1] | 3-{[(2-cyclopropyl- 1′,1′-dioxido-1-{[3- (trifluoromethoxy) phenyl]sulfonyl}- 1,2,2′,3′,5′,6′- hexahydrospiro [indole-3,4′- thiopyran]-5- yl)carbonyl]amino} benzoic acid | ¹H-NMR (300 MHz, DMSO-d6): Shift [ppm] = 0.18-0.31 (m, 1H), 0.37-0.49 (m, 1H), 0.49-0.68 (m, 2H), 0.81-0.93 (m, 1H), 0.93-1.09 (m, 1H), 1.53 (dt, 1H), 3.68 (dt, 1H), 4.40 (d, 1H), 7.48 (t, 1H), 7.63-8.07 (m, 9H), 8.33 (s, 1H), 10.34 (s, 1H). UPLC-MS (ESI+): [M + H]+ = 665. | prepared by saponification of Ex. 82 according to GP 7 |
| 84 | 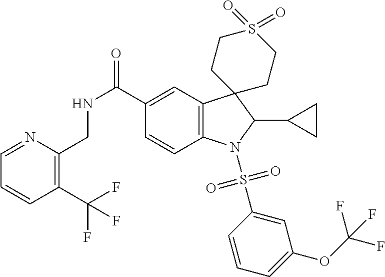 [SMILES: O=C(NCc1ncccc1C(F)(F)F)c1ccc2c(c1)C1(CCS(=O)(=O)CC1)C(C1CC1)N2S(=O)(=O)c1cccc(OC(F)(F)F)c1] | 2-cyclopropyl-1-{[3- (trifluoromethoxy) phenyl]sulfonyl)-N- {[3-(trifluoromethyl) pyridin- 2-yl]methyl}- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.19-0.29 (m, 1H), 0.38-0.48 (m, 1H), 0.50-0.66 (m, 2H), 0.80-0.90 (m, 1H), 0.96-1.06 (m, 1H), 1.47 (dt, 1H), 3.65 (dt, 1H), 4.37 (d, 1H), 4.74 (d, 2H), 7.50-7.97 (m, 9H), 8.18 (d, 1H), 8.78 (d, 1H), 9.05 (t, 1H). UPLC-MS (ESI+): [M + H]+ = 704. | from F.5 and 1-[3- (trifluoro- methyl) pyridin-2-yl] methanamine according to GP 9.1 |
| 85 | 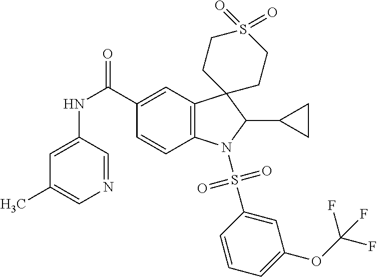 [SMILES: Cc1cncc(NC(=O)c2ccc3c(c2)C2(CCS(=O)(=O)CC2)C(C2CC2)N3S(=O)(=O)c2cccc(OC(F)(F)F)c2)c1] | 2-cyclopropyl-N-(5- methylpyridin-3-yl)- 1-{[3- (trifluoromethoxy) phenyl]sulfonyl}- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.21-0.33 (m, 1H), 0.37-0.49 (m, 1H), 0.49-0.68 (m, 2H), 0.81-0.93 (m, 1H), 0.93-1.09 (m, 1H), 1.53 (dt, 1H), 2.31 (s, 3H), 3.68 (dt, 1H), 4.41 (d, 1H), 7.63-8.03 (m, 8H), 8.17 (m, 1H), 8.69 (d, 1H), 10.28 (s, 1H). UPLC-MS (ESI+): [M + H]+ = 636. | from F.5 and 5-methyl- pyridin- 3-amine according to GP 9.2 |
| 86 | 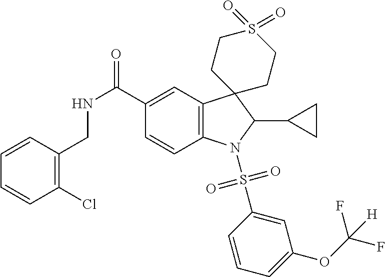 [SMILES: O=C(NCc1ccccc1Cl)c1ccc2c(c1)C1(CCS(=O)(=O)CC1)C(C1CC1)N2S(=O)(=O)c1cccc(OC(F)F)c1] | N-(2-chlorobenzyl)- 2-cyclopropyl-1-{[3- (difluoromethoxy) phenyl]sulfonyl}- 1,2,2′,3′,5′,6′- hexahydrospiro[indole- 3,4′-thiopyran]- 5-carboxamide 1′,1′-dioxde | ¹H-NMR (300 MHz, DMSO-d6): Shift [ppm] = 0.16-0.28 (m, 1H), 0.36-0.48 (m, 1H), 0.48-0.66 (m, 2H), 0.80-0.92 (m, 1H), 0.92-1.06 (m, 1H), 1.47 (dt, 1H), 3.66 (dt, 1H), 4.36 (d, 1H), 4.53 (d, 2H), 7.06-7.96 (m, 12H), 9.05 (t, 1H). UPLC-MS (ESI+): [M + H]+ = 651/653 (Cl isotope pattern). | prepared by carbonylation of intermediate D.9 with 1-(2- chlorophenyl) methanamine accordng to GP 10 |
| 87 | 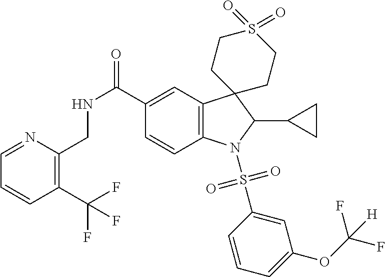 [SMILES: O=C(NCc1ncccc1C(F)(F)F)c1ccc2c(c1)C1(CCS(=O)(=O)CC1)C(C1CC1)N2S(=O)(=O)c1cccc(OC(F)F)c1] | 2-cyclopropyl-1-{[3- (difluoromethoxy) phenyl]sulfonyl}- N-{[3- (trifluoromethyl) pyridin-2-yl]methyl}- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.19-0.29 (m, 1H), 0.37-0.47 (m, 1H), 0.48-0.65 (m, 2H), 0.80-0.91 (m, 1H), 0.94-1.05 (m, 1H), 1.46 (dt, 1H), 3.65 (dt, 1H), 4.36 (d, 1H), 4.73 (d, 2H), 7.32 (tr, 1H), 7.45- 7.97 (m, 8H), 8.18 (d, 1H), 8.79 (d, 1H), 9.04 (t, 1H). UPLC-MS (ESI+): [M + H]+ = 686. | from F.6 and 1-[3- (trifluoro- methyl) pyridin-2-yl] methanamine according to GP 9.1 |
| 88 | 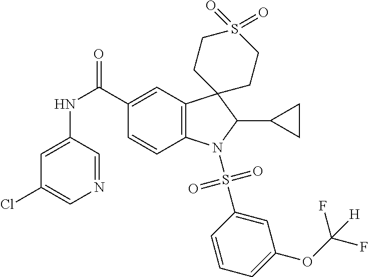 [SMILES: O=C(Nc1cncc(Cl)c1)c1ccc2c(c1)C1(CCS(=O)(=O)CC1)C(C1CC1)N2S(=O)(=O)c1cccc(OC(F)F)c1] | N-(5-chloropyridin- 3-yl)-2-cyclopropyl- 1-{[3- (difluoromethoxy) phenyl]sulfonyl}- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.21-0.30 (m, 1H), 0.38-0.48 (m, 1H), 0.50-0.66 (m, 2H), 0.83-0.92 (m, 1H), 0.94-1.05 (m, 1H), 1.52 (dt, 1H), 3.68 (dt, 1H), 4.41 (d, 1H), 7.32 (t, 1H), 7.60-7.71 (m, 4H), 7.90 (m, 1H), 7.97-8.02 (m, 1H), 8.33 (m, 1H), 8.37 (m, 1H), 8.83 (m, 1H), 10.52 (s, 1H). UPLC-MS (ESI+): [M + H]+ = 638/640 (Cl isotope pattern) | from F.6 and 5-chloro- pyridin- 3-amine according to GP 9.2 |
| 89 | 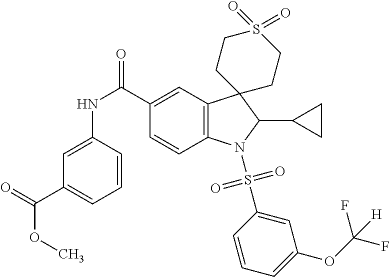 [SMILES: COC(=O)c1cccc(NC(=O)c2ccc3c(c2)C2(CCS(=O)(=O)CC2)C(C2CC2)N3S(=O)(=O)c2cccc(OC(F)F)c2)c1] | methyl 3-{[(2- cyclopropyl-1-{[3- (difluoromethoxy) phenyl]sulfonyl}- 1′,1′-dioxido- 1,2,2′,3′,5′,6′- hexahydrospiro[indole- 3,4′-thiopyran]- yl)carbonyl]amino} benzoate | ¹H-NMR (300 MHz, DMSO-d6): Shift [ppm] = 0.20-0.33 (m, 1H), 0.36-0.50 (m, 1H), 0.50-0.68 (m, 2H), 0.83-0.94 (m, 1H), 0.95-1.07 (m, 1H), 1.53 (dt, 1H), 3.67 (dt, 1H), 3.87 (s, 3H), 4.40 (d, 1H), 7.32 (t, 1H), 7.45- 7.74 (m, 7H), 7.87-8.08 (m, 3H), 8.37 (m, 1H), 10.36 (s, 1H). UPLC-MS (ESI+): [M + H]+ = 661. | from F.6 and methyl 3- amino- benzoate according to GP 9.1 |
| 89.1 | Enantiomer 1 of Ex. 89 | | Rt = 3.60 min | HPLC |
| 89.2 | Enantiomer 2 of Ex. 89 | | Rt = 4.93 min | method C |
| | | | | with column: |
| | | | | Chiralpak IA |
| | | | | 3 μm |
| | | | | 100 × 4.6 |
| | | | | mm; Solvent |
| | | | | CO₂/Ethanol |
| | | | | +0.2% vol. |
| | | | | Et2NH 80:20 |
| | | | | (v/v); |
| | | | | Detection: |
| | | | | DAD 254 nm |
| 90.1 | 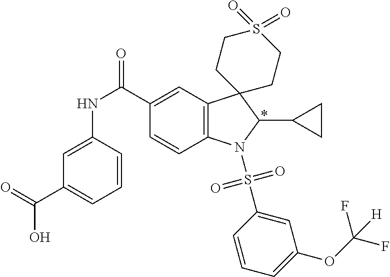 [SMILES: O=C(O)c1cccc(NC(=O)c2ccc3c(c2)C2(CCS(=O)(=O)CC2)C(C2CC2)N3S(=O)(=O)c2cccc(OC(F)F)c2)c1] | 3-{[(2-cyclopropyl- 1-{[3- (difluoromethoxy) phenyl]sulfonyl}- 1′,1′-dioxido- 1,2,2′,3′,5′,6′- hexahydrospiro[indole- 3,4′-thiopyran]- 5-yl)carbonyl]amino} benzoic acid | ¹H-NMR (300 MHz, DMSO-d6): Shift [ppm] = 0.18-0.30 (m, 1H), 0.37-0.49 (m, 1H), 0.49-0.66 (m, 2H), 0.81-0.94 (m, 1H), 0.94-1.07 (m, 1H), 1.52 (dt, 1H), 3.68 (dt, 1H), 4.40 (d, 1H), 7.33 (t, 1H), 7.47-7.72 (m, 7H), 7.88-8.04 (m, 3H), 8.33 (s, 1H), 10.34 (s, 1H). UPLC-MS (ESI+): [M + H]+ = 647. | prepared by saponification of Ex. 89.1 according to GP 7 |
| 90.2 | Enantiomer 2 of Ex. 90.1 | 3-{[(2-cyclopropyl- | | saponification |
| | | 1-([3- | | of Ex. 89.2 |
| | | (difluoromethoxy) | | according to |
| | | phenyl]sulfonyl}- | | GP 7 |
| | | 1′,1′-dioxido- | | |
| | | 1,2,2′,3′,5′,6′- | | |
| | | hexahydrospiro[indole- | | |
| | | 3,4′-thiopyran]- | | |
| | | 5-yl)carbonyl]amino} | | |
| | | benzoic acid | | |
| 91 | 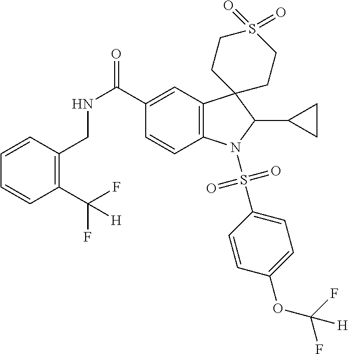 [SMILES: O=C(NCc1ccccc1C(F)F)c1ccc2c(c1)C1(CCS(=O)(=O)CC1)C(C1CC1)N2S(=O)(=O)c1ccc(OC(F)F)cc1] | 2-cyclopropyl-1-{[4- (difluoronnethoxy) phenyl]sulfonyl}-N- [2-(difluoromethyl) benzyl]- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.15-0.28 (m, 1H), 0.37-0.66 (m, 3H), 0.80-0.91 (m, 1H), 0.92-1.05 (m, 1H), 1.45 (dt, 1H), 3.61 (dt, 1H), 4.35 (d, 1H), 4.61 (d, 2H), 7.08-7.94 (m, 13H), 9.05 (t, 1H). UPLC-MS (ESI+): [M + H]+ = 667. | from F.7 and 1-[2-(difluoro- methyl) phenyl] methanamine according to GP 9.1 |
| 92 | 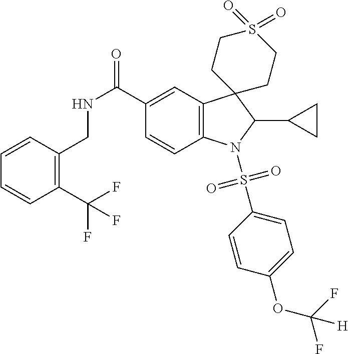 [SMILES: O=C(NCc1ccccc1C(F)(F)F)c1ccc2c(c1)C1(CCS(=O)(=O)CC1)C(C1CC1)N2S(=O)(=O)c1ccc(OC(F)F)cc1] | 2-cyclopropyl-1-{[4- (difluoromethoxy) phenyl]sulfonyl}-N- [2-(trifluoromethyl) benzyl]- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.18-0.30 (m, 1H), 0.36-0.67 (m, 3H), 0.80-0.92 (m, 1H), 0.93-1.07 (m, 1H), 1.47 (dt, 1H), 3.62 (dt, 1H), 4.36 (d, 1H), 4.65 (d, 2H), 7.08-7.96 (m, 12H), 9.09 (t, 1H). UPLC-MS (ESI+): [M + H]+ = 685. | from F.7 and 1-[2- (trifluoro- methyl) phenyl] methanamine according to GP 9.1 |
| 93 | 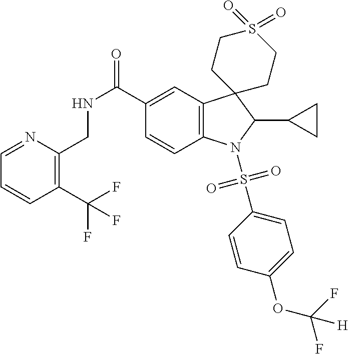 [SMILES: O=C(NCc1ncccc1C(F)(F)F)c1ccc2c(c1)C1(CCS(=O)(=O)CC1)C(C1CC1)N2S(=O)(=O)c1ccc(OC(F)F)cc1] | 2-cyclopropyl-1-{[4- (difluoromethoxy) phenyl]sulfony}-N- {[3-(trifluoromethyl) pyridin- 2-yl]methyl}- 1,2,2′,3′,5′,6′- hexahydrospiro[indole- 3,4′-thiopyran]- 5-carboxamide 1′,1′-dioxide | ¹H-NMR (300 MHz, DMSO-d6): Shift [ppm] = 0.15-0.28 (m, 1H), 0.35-0.66 (m, 3H), 0.79-0.91 (m, 1H), 0.93-1.08 (m, 1H), 1.45 (dt, 1H), 3.61 (dt, 1H), 4.35 (d, 1H), 4.73 (d, 2H), 7.07-7.66 (m, 5H), 7.79-7.94 (m, 4H), 8.18 (d, 1H), 8.78 (d, 1H), 9.04 (t, 1H). UPLC-MS (ESI+): [M + H]+ = 686. | from F.7 and 1-[3- (trifluoro- methyl) pyridin-2-yl] methanamine according to GP 9.1 |
| 94 | 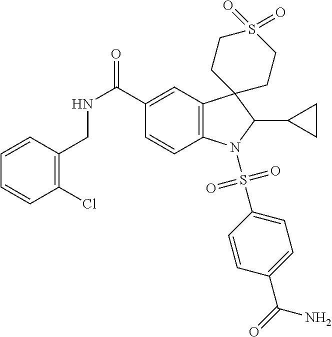 [SMILES: NC(=O)c1ccc(S(=O)(=O)N2c3ccc(C(=O)NCc4ccccc4Cl)cc3C3(CCS(=O)(=O)CC3)C2C2CC2)cc1] | 1-[(4-carbamoylphenyl) sulfonyl]- N-(2-chlorobenzyl)- 2-cyclopropyl- 1,2,2′,3′,5′,6′- hexahydrospiro [indole-3,4′- thiopyran]- 5-carboxamide 1′,1′-dioxide | ¹H-NMR (300 MHz, DMSO-d6): Shift [ppm] = 0.16-0.20 (m, 1H), 0.38-0.48 (m, 1H), 0.49-0.65 (m, 2H), 0.83-0.91 (m, 1H), 0.96-1.05 (m, 1H), 1.38-1.48 (m, 1H), 2.48-2.57 (m, 3H), 3.20-3.25 (m, 2H), 3.59-3.69 (m, 1H), 4.38 (d, 1H), 4.53 (d, 2H), 7.26-7.37 (m, 3H), 7.43- 7.46 (m, 1H), 7.60-7.63 (m, 2H), 7.83 (d, 1H), 7.88-7.96 (m, 5H), 8.11 (br. s., 1H), 9.02 (t, 1H). UPLC-MS (ESI+): [M + H]+ = 628/630 (Cl isotope pattern). | prepared by carbonylation of intermediate D.11 with 1-(2- chlorophenyl) methanamine according to GP 10 |
| 95 | 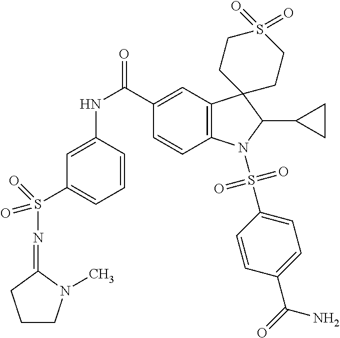 [SMILES: CN1CCCC1=NS(=O)(=O)c1cccc(NC(=O)c2ccc3c(c2)C2(CCS(=O)(=O)CC2)C(C2CC2)N3S(=O)(=O)c2ccc(C(N)=O)cc2)c1] | 1-[(4- carbamoylphenyl) sulfonyl]-2- cyclopropyl-N-{3- [(1-methylpyrrolidin- 2-ylidene) sulfamoyl]phenyl}- 1,2,2′,3′,5′,6′- hexahydrospiro [indole-3,4′-thiopyran]- 5-carboxamide 1′,1′-dioxide | ¹H-NMR (400 MHz, DMSO-d6): Shift [ppm] = 0.18-0.23 (m, 1H), 0.40-0.47 (m, 1H), 0.51-0.58 (m, 1H), 0.59-0.66 (m, 1H), 0.85-0.91 (m, 1H), 0.97-1.06 (m, 1H), 1.48 (dt, 1H), 1.90- 2.00 (m, 2H), 2.50-2.64 (m, 3H), 2.85-2.87 (m, 2H), 2.89 (s, 3H), 3.23-3.25 (m, 2H), 3.46- 3.49 (m, 2H), 3.66 (dt, 1H), 4.41 (d, 1H), 7.49-7.51 (m, 2H), 7.61 (br. s., 1H), 7.66 (d, 1H), 7.87 (d, 1H), 7.90-7.99 (m, 6H), 8.12 (br. s., 1H), 8.22 (br. s., 1H), 10.37 (s, 1H). UPLC-MS (ESI+): [M + H]+ = 740. | from F.10 and 3-amino- N-(1- methyl- pyrrolidin- 2-ylidene) benzene- sulfonamide according to GP 9.1 |
| 96 | 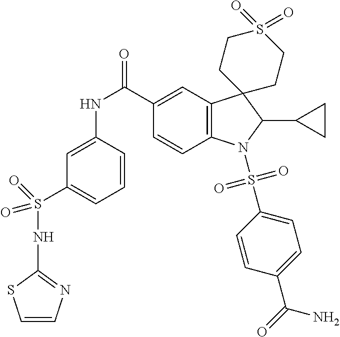 [SMILES: NC(=O)c1ccc(S(=O)(=O)N2c3ccc(C(=O)Nc4cccc(S(=O)(=O)Nc5nccs5)c4)cc3C3(CCS(=O)(=O)CC3)C2C2CC2)cc1] | 1-[(4- carbamoylphenyl) sulfonyl]-2- cyclopropyl-N-[3- (1,3-thiazol-2- ylsulfamoyl)phenyl]- 1,2,2′,3′,5′,6′- hexahydrospiro[indole- 3,4′-thiopyran]- 5-carboxamide 1′,1′-dioxide | ¹H-NMR (400 MHz, DMSO-d6): Shift [ppm] = 0.18-0.22 (m, 1H), 0.40-0.47 (m, 1H), 0.52-0.65 (m, 2H), 0.85-0.91 (m, 1H), 0.97-1.05 (m, 1H), 1.48 (dt, 1H), 2.52-2.65 (m, 3H), 3.19- 3.25 (m, 2H), 3.66 (dt, 1H), 4.41 (d, 1H), 6.81 (d, 1H), 7.23 (d, 1H), 7.47-7.53 (m, 2H), 7.60 (br. s., 1H), 7.66 (d, 1H), 7.87 (d, 1H), 7.90-7.99 (m, 6H), 8.12- 8.13 (m, 1H), 8.26 (br. s., 1H), 10.38 (s, 1H), 12.73 (br. s., 1H). UPLC-MS (ESI+): [M + H]+ = 742. | from F.10 and 3-amino- N-(1,3- thiazol-2-yl) benzene- sulfonamide according to GP 9.1 |

### Example 97

### N-{2-Cyclopropyl-1-[(4-fluorophenyl)sulfonyl]-1′,1′-dioxido-1,2,2′,3′,5′,6′-hexahydrospiro[indole-3,4′-thiopyran]-5-yl}cyclopropanecarboxamide

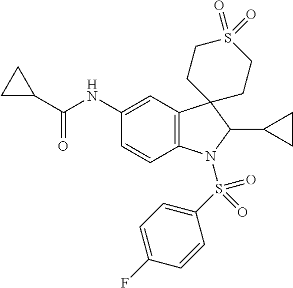 [SMILES: O=C(Nc1ccc2c(c1)C1(CCS(=O)(=O)CC1)C(C1CC1)N2S(=O)(=O)c1ccc(F)cc1)C1CC1]

According to GP 15.1 111 μmol of intermediate I.1 and 166 μmol cyclopropanecarboxylic acid were reacted with 170 μmol HATU in the presence of 23 μL (170 μmol) triethylamine in 2 mL of DMF to yield 58 mg (100%) of the desired amide. ¹H-NMR (300 MHz, DMSO-d6): Shift [ppm]=0.18 (d, 1H), 0.31-0.48 (m, 2H), 0.49-0.59 (m, 1H), 0.69-0.84 (m, 5H), 0.88-1.02 (m, 1H), 1.27-1.39 (m, 1H), 1.64-1.72 (m, 1H), 2.32-2.42 (m, 2H), 2.42-2.55 (m, 1H), 3.12-3.19 (m, 2H), 3.52 (dt, 1H), 4.21 (d, 1H), 7.31-7.44 (m, 4H), 7.49 (s, br, 1H), 7.77-7.82 (m, 2H), 10.16 (s, 1H); UPLC-MS (ESI+): [M+H]⁺=519.

TABLE 3
The following examples were prepared in analogy to example 97 starting from the
aniline intermediate I.1 and commercially available carboxylic acids, applying the
indicated general procedure.

| No | Structure | Name | Analytical data | Methods |
|---|---|---|---|---|
| 98 | 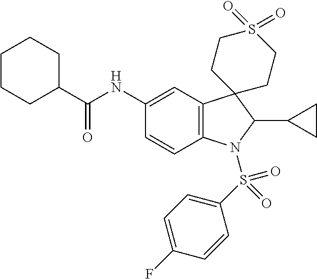 [SMILES: O=C(Nc1ccc2c(c1)C1(CCS(=O)(=O)CC1)C(C1CC1)N2S(=O)(=O)c1ccc(F)cc1)C1CCCCC1] | N-{2-cyclopropyl- 1-[(4- fluorophenyl) sulfonyl]- 1′,1′-dioxido- 1,2,2′,3′,5′,6′- hexahydrospiro [indole-3,4′- thiopyran]-5- yl}cyclohexane- carboxamide | ¹H-NMR (300 MHz, DMSO-d6): Shift [ppm] = 0.14 (d, 1H), 0.31-0.47 (m, 2H), 0.48-0.58 (m, 1H), 0.75-0.84 (m, 1H), 0.88-1.00 (m, 1H), 1.09- 1.41 (m, 6H), 1.56-1.64 (m, 1H), 1.66- 1.77 (m, 4H), 2.18-2.41 (m, 3H), 2.42-2.55 (m, 1H), 3.11-3.20 (m, 2H), 3.53 (dt, 1H), 4.21 (d, 1H), 7.31- 7.40 (m, 3H), 7.45 (dd, 1H), 7.50 (d, 1H), 7.77-7.81 (m, 2H), 9.79 (s, 1H); UPLA-MS (ESI+): [M + H]⁺ = 561. | GP 15.1 |
| 99 | 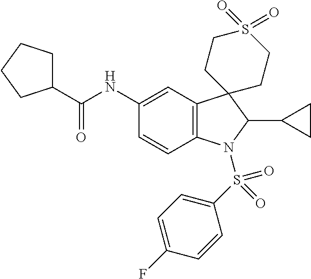 [SMILES: O=C(Nc1ccc2c(c1)C1(CCS(=O)(=O)CC1)C(C1CC1)N2S(=O)(=O)c1ccc(F)cc1)C1CCCC1] | N-{2-cyclopropyl- 1-[(4- fluorophenyl) sulfonyl]- 1′,1′-dioxido- 1,2,2′,3′,5′,6′- hexahydrospiro [indole-3,4′- thiopyran]-5- yl}cyclohexane- carboxamide | ¹H-NMR (300 MHz, DMSO-d6): Shift [ppm] = 0.15 (d, 1H), 0.31-0.47 (m, 2H), 0.48-0.59 (m, 1H), 0.76-0.83 (m, 1H), 0.88-1.00 (m, 1H), 1.32 (dt, 1H), 1.46-1.83 (m, 9H), 2.25-2.34 (m, 1H), 2.40-2.54 (m, 1H), 2.64- 2.72 (m, 1H), 3.11-3.19 (m, 2H), 3.53 (dt, 1H), 4.22 (d, 1H), 7.31-7.41 (m, 3H), 7.44 (dd, 1H), 7.50 (d, 1H), 7.77- 7.82 (m, 2H), 9.85 (s, 1H); UPLC-MS (ESI+): [M + H]⁺ = 547. | GP 15.1 |

### Biological Assays

### 1. Materials

Buserelin was purchased from Welding (Frankfurt/Main, Germany) or USbiological (#B8995, Swampscott, USA) for IP-One HTRF® assays and LHRH from Sigma-Aldrich® (Munich, Germany). Labelled cells, Tag-Lite buffer, labelled and unlabelled GnRHR binding peptide for Tag-Lite® binding assay was purchased by Cisbio Bioassays (Bagnols-sur-Cèze Cedex, France). The radio labelling was performed in the Department of Isotope Chemistry of Bayer Schering Pharma AG (Berlin, Germany) by the iodogen method using [¹²⁵I]sodium iodide (2000 Ci/mmol; PerkinElmer Life and Analytical Sciences, USA) yielding [¹²⁵I]monoiodo-buserelin. The radio-tracer was purified by reversed phase HPLC on a Spherisorb ODS II column (250×4 mm, particle size 3 μm) by elution with acetonitrile/water (34:66) containing 39 mM trifluoracetic acid at a flow rate of 1 mL/min. The retention time of [¹²⁵I]monoiodo-buserelin was approximately 17 min. All other chemicals were obtained from commercial sources at the highest purity grade available.

### 2. Methods

### 2.1. Receptor Binding Assay Using Radiolabelled Buserelin

Binding studies for competition curves were run in triplicate samples in 96 well polypropylene microtiter plates (Nunc, New Jersey, USA). One assay sample contained 70 μl of 300,000 cells for CHO cells stably transfected with the human GnRH receptor, 20 μl of ¹²⁵I-labelled buserelin (100,000 cpm per sample for competition curves) and 10 μl of assay buffer or test compound solution. Test compounds were dissolved in DMSO. Cetrorelix was dissolved in 0.1 M hydrochloric acid. Serial dilutions (5×10⁻⁶ M to 5−10⁻¹² M) were prepared in assay buffer (DMEM or DMEM/Ham's F12 medium, 10 mM Hepes buffer pH 7.5, 0.5% BSA). Nonspecific binding was determined in presence of excess unlabelled buserelin (10-5 M). Test samples were incubated for 60 min at room temperature. Bound and free ligand were separated by filtration over Unifilter GF/C filter microtiter plates (PerkinElmer, CT, USA) by applying negative pressure and washing twice with 200 mL of 0.02 M Tris/hydrochloric acid, pH 7.4. The filter plates were soaked with 0.3% polyethylenimine (Serva; Heidelberg, Germany) for 30 min prior to use in order to reduce nonspecific binding. The radioactivity retained by the filters was determined in a TopCount NXT HTS (PerkinElmer, CT, USA) using 20 μl/well MicroScint40 scintillator cocktail (PerkinElmer, CT, USA). Competition curves were obtained by plotting the measured radioactivity against the respective test compound concentration by using an in-house software.

### 2.2. Tag-Lite® Receptor Binding Assay

This binding assay is based on the fluorescence resonance energy transfer between fluorescence donor labelled human GnRHR and a green-labelled GnRHR binding peptide. Compounds interfering with the ligand binding side of the human GnRHR will replace the labelled peptide resulting in a signal decrease. The assay principle was established by Cisbio Bioassays (Bagnols-sur-Cèze Cedex, France) and further details are available on their homepage.

The assay procedure was further optimized for use in-house with reduced assay volumes. Frozen Hek293 cells, transiently transfected with human GnRHR and Terbium-labelling of the receptor, were supplied by Cisbio Bioassays as well as Tag-Lite buffer and green-labelled GnRHR binding peptide. Cells were thawed and transferred to cold Tag-Lite buffer. A volume of 8 μl of this cell suspension were added to 100 nl of a 160-fold concentrated solution of the test compound in DMSO pre-dispensed in a well of a white low-volume 384-well microtiter plate (Greiner Bio-One, Frickenhausen, Germany). The mixture was incubated for 5 min at room temperature. In the next step either 4 μl Tag-Lite buffer or as control 4 μl of an exceeding amount unlabelled binding peptide in Tag-Lite buffer were transferred to the mixture. The green-labelled GnRHR binding peptide was added in a final step at EC₅₀ in a volume of 4 μl Tag-Lite buffer. After an incubation of 1 h at room temperature plates were measured in a microplate reader, e.g. a PHERAstar (BMG Labtechnologies, Offenburg, Germany) by using a specific optic module.

A ratio from the fluorescence emissions at 520 nm (green fluorescence) and at 490 nm (background signal of Terbium-labelled GnRHR) was calculated and the data were normalized (reaction without test compound=0% inhibition of binding of green-labelled peptide; reaction without test compound with exceeding amount unlabelled binding peptide=100% inhibition of binding of green-labelled peptide). On the same microtiter plate, compounds were tested at 10 different concentrations in the range of 12.5 μM to 0.64 nM (12.5 μM, 4.2 μM, 1.4 μM, 0.46 μM, 0.15 μM, 51 nM, 17 nM, 5.7 nM, 1.9 nM and 0.64 nM; dilution series prepared before the assay at the level of the 160-fold conc. stock solutions by serial 1:3 dilutions in 100% DMSO) in duplicate values for each concentration. By using an in-house software, the IC₅₀ values were calculated by a 4 parameter fit.

2.3. IP-One HTRF® Assay By using homogenous time-resolved fluorescence resonance energy transfer (HTRF), the generation of one component of the GnRH—R signalling cascade can be measured. After stimulation of CHO cells stably expressing human GnRH receptor (established by Prof. Thomas Gudermann, currently University of Marburg, Germany; supplied as frozen cell aliquots by Cell Culture Services, Hamburg, Germany) with the EC₈₀ of the GnRH agonist buserelin, Gq protein-coupled receptor signalling cascade is activated resulting in PLC-dependent cleavage of PIP2 to Inositol-1,4,5-triphosphate (IP3) and Diacylglycerol. The second messenger IP3 is degraded intracellularly to myo-inositol. Inhibition of the final degradation step from Inositol-1-phosphate (IP1) to myo-inositol by addition of lithium chloride leads to accumulation of IP1 in the cells. In cell lysates, IP1 can be detected via an antibody-based HTRF detection technology, where IP1 can displace the FRET acceptor IP1-d2 from binding by Terbium-labelled anti-IP1 antibody as donor resulting in a signal decrease. Compounds were tested for their capability of inhibiting GnRH—R activation by buserelin. For all IP-One HTRF® assays reagents of Cisbio Bioassays (IP-One Tb Jumbo kit, #62IPAPEJ; Cisbio Bioassays, Bagnols sur Cèze Cedex, France) were used.

For the assay, frozen cell aliquots were thawed and a cell suspension (3.33×10⁶ cells/mL) containing IP1-d2 (dilution 1:40) was prepared and incubated at 37° C. After 1 h 3 μl of the cell suspension were added to 50 nl of a 100-fold concentrated solution of the test compound in DMSO pre-dispensed in a well of a white low-volume 384-well microtiter plate (Greiner Bio-One, Frickenhausen, Germany). The mixture was incubated for 20 min at 22° C. to allow for pre-binding of the test compound to the GnRH—R. The receptor signaling cascade was stimulated by addition of 2 μl buserelin or LHRH (at EC₅₀ or EC₈₀) in stimulation buffer (10 mM Hepes pH 7.4, 1 mM CaCl2, 0.5 mM MgCl2, 4.2 mM KCl, 146 mM NaCl, 5.5 mM α-D-Glucose, 0.05% BSA, 125 mM LiCl (final assay concentration 50 mM) in aqua dest.). Plates were incubated for 1 h at 37° C. and 5% carbon dioxide before the cells were lysed by adding 3 μl Terbium-labelled anti-IP1 antibody (1:40) diluted in Conjugate & Lysis buffer as supplied with the kit. After an incubation for 1 h at 22° C. to enable complete cell lysis and antibody binding to free IP1 or IP1-d2, plates were measured in an HTRF reader, e.g. a RUBYstar, PHERAstar (both BMG Labtechnologies, Offenburg, Germany) or a Viewlux (PerkinElmer LAS, Rodgau-Jügesheim, Germany).

From the fluorescence emissions at 665 nm (FRET) and at 620 nm (background signal of Terbium-antibody), the ratio (emission at 665 nm divided by emission at 620 nm) was calculated and the data were normalized (reaction without test compound=0% inhibition; all other assay components except agonist=100% inhibition). On the same microtiter plate, compounds were tested at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM; dilution series prepared before the assay at the level of the 100-fold conc. stock solutions by serial 1:3 dilutions in 100% DMSO) in duplicate values for each concentration. By using an in-house software, the IC₅₀ values were calculated by a 4 parameter fit.

### 2.4. LH Suppression in the Ovariectomized Rat

The in vivo potency of GnRH antagonists can be quantified by a LH suppression test in ovariectomized rats. GnRH triggers the LH release from the pituitary mediated by GnRH receptors. Ovarectomy of adult female rats results in elevated levels of circulating LH due to a lack of negative feedback by gonadal steroids. GnRH antagonists suppress the release of LH and accordingly suppression of LH levels can be used to quantify the in vivo potency of GnRH antagonists.

Female adult rats were ovariectomized surgically and they were allowed to recover for at least one week. The animals received 1 mg/kg, 10 mg/kg or 30 mg/kg of Example 14.1 by single per oral administration. For comparison reasons a vehicle control and a positive control, 0.1 mg/kg cetrorelix i.p., were given once. At 0 min, 15 min, 30 min, 1 hour, 2 hours, 6 hours and 24 hours after compound administration blood was taken from the retro orbital plexus (n=6 per blood withdrawal) for the measurement of serum LH and serum compound levels.

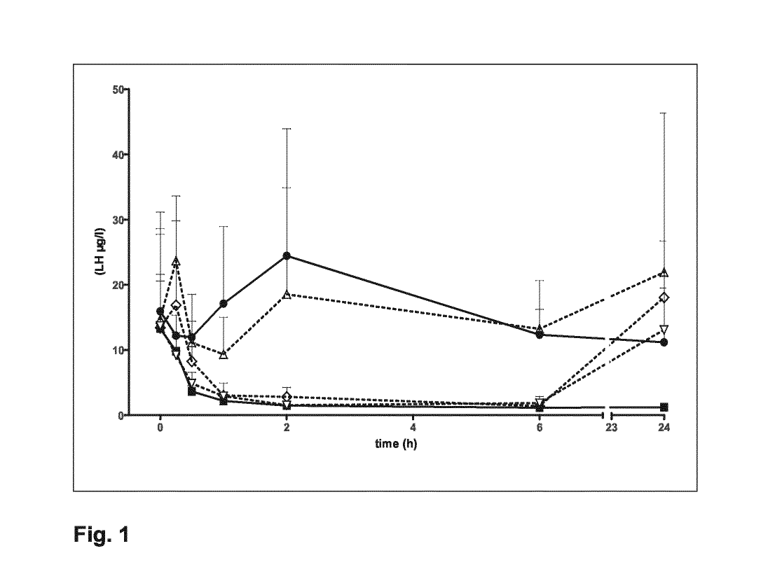

Per oral administration of Example 14.1 to ovariectomized rats resulted in a LH suppression of 11% (1 mg/kg), 89% (10 mg/kg) and 88% (30 mg/kg) at 6 hours following administration (see FIG. 1). Similarly, the positive control 0.1 mg/kg cetrorelix (i.p.) suppressed the LH level by 91% at 6 hours. At 6 hours the compound levels increased to 0.04±μM 0.02 (1 mg/kg), 0.77±0.2 μM (10 mg/kg) and 1.84±0.53 μM (30 mg/kg).

To conclude Example 14.1 is an orally active GnRH antagonist in vivo.

### FIGURES

As nonbinding explanatory example of compounds according to the invention FIG. 1 represents the LH level following administration of the compound according to Example 14.1, to ovariectomized adult rats. [Filled circle: Vehicle; Filled square: Cetrorelix (0.1 mg/kg); Open reversed triangle: Example 14.1 (30 mg/kg); Open diamond: Example 14.1 (10 mg/kg); Open triangle: Example 14.1 (1 mg/kg)]. Values are given as mean±standard deviation (n=6).

### Results

The data reveal that the compounds of the present invention have antagonist activities on the human GnRH receptor.

Within the meaning of the present invention the antagonist activity is reflected by the ability of a compound of the invention to antagonize human GnRH receptor stimulation in IP-One HTRF® assay at least three times the standard deviation over the background level.

TABLE 4
Potency in receptor binding assay using TAG-LITE ®
technology; the potency is given as IC₅₀ [μM].

| Example | Potency [μM] |
|---|---|
| 13.1 | 0.0085 |
| 18.1 | 0.011 |
| 24 | 0.187 |
| 14.1 | 0.020 |
| 67 | 0.154 |
| 68.1 | 0.0095 |
| 75 | 0.030 |

TABLE 5
Potency in IP-One HTRF ® assay with buserelin (at EC₈₀)
stimulation; the potency is given as IC₅₀ [μM].

| Example | Potency [μM] |
|---|---|
| 1 | 0.584 |
| 1.1 | 0.146 |
| 1.2 | 19.5 |
| 2 | 0.189 |
| 2.1 | 9.32 |
| 2.2 | 0.083 |
| 3 | 0.584 |
| 3.1 | 0.226 |
| 3.2 | >20.0 |
| 4 | 0.281 |
| 5 | 0.345 |
| 6 | 6.72 |
| 7 | 1.06 |
| 8 | 2.51 |
| 9 | 4.83 |
| 10 | 1.39 |
| 11 | 0.378 |
| 12 | 0.186 |
| 12.1 | 0.178 |
| 12.2 | 3.73 |
| 13 | 0.030 |
| 13.1 | 0.012 |
| 13.2 | 0.383 |
| 14 | 0.245 |
| 14.1 | 0.104 |
| 14.2 | 5.39 |
| 15 | 0.103 |
| 15.1 | >20.0 |
| 15.2 | 0.069 |
| 16 | 0.074 |
| 16.1 | 0.062 |
| 16.2 | 6.84 |
| 17 | 0.184 |
| 17.1 | 0.139 |
| 17.2 | 2.48 |
| 18 | 0.047 |
| 18.1 | 0.026 |
| 18.2 | 5.24 |
| 19 | 4.17 |
| 20 | 0.346 |
| 21 | 3.04 |
| 22 | 1.48 |
| 23 | 0.328 |
| 23.1 | 1.87 |
| 23.2 | 0.153 |
| 24 | 0.264 |
| 25 | 2.62 |
| 26 | 0.646 |
| 27 | 9.02 |
| 28 | 0.405 |
| 29 | >20.0 |
| 30 | 0.245 |
| 30.1 | 0.441 |
| 30.2 | 0.190 |
| 31 | 0.503 |
| 31.1 | 0.266 |
| 31.2 | 15.1 |
| 32 | 0.058 |
| 32.1 | 0.034 |
| 32.2 | 1.26 |
| 33 | 0.958 |
| 33.1 | 0.425 |
| 33.2 | >20.0 |
| 33.3 | >20.0 |
| 34 | 0.066 |
| 34.1 | 0.038 |
| 34.2 | 0.825 |
| 35 | 0.627 |
| 35.1 | 0.394 |
| 35.2 | >20.0 |
| 36 | 0.169 |
| 36.1 | 0.107 |
| 36.2 | 3.89 |
| 37 | 0.078 |
| 37.1 | 2.13 |
| 37.2 | 0.044 |
| 38 | 0.136 |
| 38.1 | 0.088 |
| 38.2 | 6.86 |
| 39 | 0.053 |
| 39.1 | 0.040 |
| 39.2 | 0.565 |
| 40 | 0.032 |
| 40.1 | 0.019 |
| 40.2 | 0.632 |
| 41 | 0.234 |
| 41.1 | 0.102 |
| 41.2 | 0.428 |
| 42 | 0.096 |
| 42.1 | 0.437 |
| 42.2 | 0.099 |
| 43 | 0.056 |
| 43.1 | 0.344 |
| 43.2 | 0.036 |
| 44 | 0.031 |
| 44.1 | 0.025 |
| 44.2 | 0.302 |
| 45 | 0.299 |
| 45.1 | 0.253 |
| 45.2 | 4.46 |
| 46 | 0.081 |
| 46.1 | 0.050 |
| 46.2 | 0.732 |
| 47 | 0.133 |
| 47.1 | 0.073 |
| 47.2 | 8.87 |
| 48 | 0.055 |
| 49.1 | 0.040 |
| 49.2 | 0.109 |
| 50 | 0.054 |
| 51.1 | 0.080 |
| 51.2 | 1.18 |
| 52 | 0.752 |
| 53 | 0.617 |
| 54 | 0.091 |
| 55 | 0.622 |
| 56 | 0.136 |
| 57 | 0.071 |
| 58 | 0.041 |
| 59 | 0.080 |
| 60 | 0.192 |
| 60.1 | 0.055 |
| 60.2 | 1.14 |
| 61.1 | 0.084 |
| 61.2 | 2.01 |
| 62 | 0.0098 |
| 62.1 | 6.43 |
| 62.2 | 0.0048 |
| 63 | 0.325 |
| 63.1 | 0.088 |
| 63.2 | 3.43 |
| 64 | 0.0093 |
| 64.1 | 0.0046 |
| 64.2 | 0.066 |
| 65 | 0.479 |
| 66 | 0.177 |
| 67 | 0.113 |
| 68 | 0.0045 |
| 68.1 | 0.0042 |
| 68.2 | 0.143 |
| 69 | 0.320 |
| 70 | 0.063 |
| 70.1 | 0.033 |
| 70.2 | 0.202 |
| 71 | 0.052 |
| 71.1 | 0.049 |
| 71.2 | 1.63 |
| 72 | 0.013 |
| 72.1 | 0.011 |
| 72.2 | 0.403 |
| 73 | 0.130 |
| 74 | 0.066 |
| 75 | 0.053 |
| 76 | 0.044 |
| 76.1 | 0.021 |
| 76.2 | 0.689 |
| 77 | 0.700 |
| 77.1 | 0.054 |
| 77.2 | 0.963 |
| 78 | 0.706 |
| 79 | 0.064 |
| 79.1 | 0.054 |
| 79.2 | 1.02 |
| 80 | 0.218 |
| 80.1 | 0.315 |
| 80.2 | >20.0 |
| 81 | 0.437 |
| 82 | 0.609 |
| 83 | 0.140 |
| 84 | 0.309 |
| 85 | 0.804 |
| 86 | 0.196 |
| 87 | 0.143 |
| 88 | 0.262 |
| 89 | 0.134 |
| 89.1 | 0.063 |
| 89.2 | >20.0 |
| 90.1 | 0.041 |
| 90.2 | 2.86 |
| 91 | 0.0059 |
| 92 | 0.031 |
| 93 | 0.053 |
| 94 | 0.222 |
| 95 | 0.225 |
| 96 | 0.484 |
| 97 | 2.23 |
| 98 | 0.039 |
| 99 | 0.300 |

1. A compound according to Formula (I) 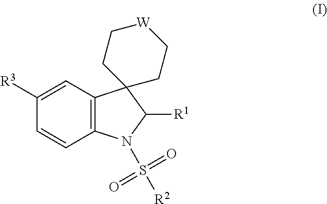
in which
W is selected from the group consisting of O, S(O)ₓ with x=0, 1 or 2;
R¹ is selected from the group consisting of hydrogen, C₁-C₆-alkyl, C₃-C₁₀-cycloalkyl, C₂-C₆-alkenyl, C₂-C₆-alkynyl, aryl, hydroxy-C₁-C₆-alkyl, C₁-C₆-alkoxy-C₁-C₆-alkyl;
R² is an aryl or heteroaryl group which can be unsubstituted or substituted one to three times with a group R⁴ selected from a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, C(O)NH₂, C(O)NH—C₁-C₆-alkyl, C(O)N(C₁-C₆-alkyl)₂ in which the two alkyl groups are independent from each other, CN;
R³ is selected from the group consisting of C(O)N(R^{5a})(R^{5b}), N(H)C(O)R⁶, N(H)C(O)N(R^{5a})(R^{5b}), or N(H)C(O)OR⁷ and
R^{5a}, R^{5b} and R⁶ are selected, independently from one another, from the group consisting of hydrogen, C₁-C₆-alkyl, C₁-C₆-haloalkyl, hydroxy-C₁-C₆-alkyl; C₂-C₆-alkenyl, C₂-C₆-alkynyl, C₁-C₆-alkoxy-C₁-C₆-alkyl, C₃-C₁₀-cycloalkyl, C₃-C₁₀-cycloalkyl-C₁-C₆-alkylen-, aryl, aryl-C₁-C₆-alkylen-, aryl-cyclopropyl, heteroaryl, heteroaryl-C₁-C₆-alkylen-, in which said cycloalkyl, aryl, heteroaryl groups are optionally substituted up to three times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other;
R⁷ is selected from the group consisting of C₁-C₆-alkyl, C₁-C₆-haloalkyl, hydroxy-C₁-C₆-alkyl, C₂-C₆-alkenyl, C₂-C₆-alkynyl, C₁-C₆-alkoxy-C₁-C₆-alkyl, C₃-C₁₀-cycloalkyl, C₃-C₁₀-cycloalkyl-C₁-C₆-alkylen-, aryl, aryl-C₁-C₆-alkylen-, heteroaryl, or heteroaryl-C₁-C₆-alkylen- in which said cycloalkyl, aryl, heteroaryl group is optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other
and the salts thereof.

2. A compound according to claim 1 characterised in that
R¹ is selected from the group consisting of C₁-C₆-alkyl, C₃-C₁₀-cycloalkyl.

3. A compound according to claim 1 wherein
R² is a phenyl.

4. A compound according to claim 1 wherein
R⁴ is a halogen, C₁-C₆-alkoxy, C₁-C₆-haloalkoxy, C(O)O—C₁-C₆-alkyl, C(O)OH, or C(O)NH₂ group.

5. A compound according to claim 1 wherein
R² is a phenyl group substituted in para with R⁴ being a fluorine or a OCF²H.

6. A compound according to claim 1 wherein
R² is a phenyl group substituted in meta with R⁴ being a C₁-C₆-alkoxy, C₁-C₆-haloalkoxy, or C(O)O—C₁-C₆-alkyl.

7. A compound according to claim 1 wherein
R³ is selected from the group consisting of C(O)NH(R^{5a}) and
R^{5a} is C₃-C₁₀-cycloalkyl, C₃-C₁₀-cycloalkyl-C₁-C₆-alkylen-, aryl, aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

8. A compound according to claim 1 wherein
R³ is N(H)C(O)R⁶, and
R⁶ is C₃-C₁₀-cycloalkyl, C₃-C₁₀-cycloalkyl-C₁-C₆-alkylen-, aryl, aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

9. A compound according to claim 1 wherein
R^{5a} is C₃-C₁₀-cycloalkyl, C₃-C₁₀-cycloalkyl-C₁-C₆-alkylen-, aryl or aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

10. A compound according to claim 1 wherein
R^{5b} is a hydrogen or C₁-C₆-alkyl, C₁-C₆-haloalkyl.

11. A compound according to claim 1 wherein
R⁶ is C₃-C₁₀-cycloalkyl, C₃-C₁₀-cycloalkyl-C₁-C₆-alkylen-, aryl, aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

12. A compound according to claim 1 wherein
R^{5a}, R⁶ and R⁷ are selected from the group consisting of cyclopropyl, cyclopropyl-CH²—, cyclopentyl, cyclopentyl-CH²—, cyclohexyl, cyclohexyl-CH²—, phenyl, phenyl-CH²—, pyridyl, pyridyl-CH²—, 3,4-dihydro-2H-chromen-4-yl, optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(CH³)².

13. A compound according to claim 1 wherein
R^{5a}, R⁶ and R⁷ are selected from the group consisting of cyclopropyl, cyclopropyl-CH²—, cyclopentyl, cyclopentyl-CH²—, cyclohexyl, cyclohexyl-CH²—, 3,4-dihydro-2H-chromen-4-yl; and phenyl, phenyl-CH²—, pyridyl, pyridyl-CH²—, substituted one or two times with a fluorine, chlorine, hydroxy, CH³, CF²H, CF³, C₁-C₆-alkoxy, C₁-C₆-haloalkoxy, C(O)OH, C(O)OCH³, CN, C(O)NH₂, S(O)₂—CH³, S(O)₂NH₂, S(O)₂N(CH³)².

14. A compound according to Formula (Ia) 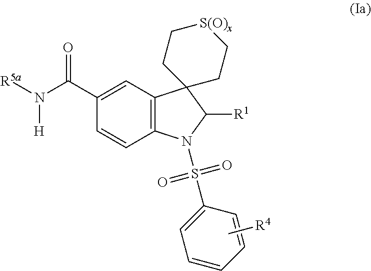
in which x=0, 1 or 2;
R¹ is selected from the group consisting of C₁-C₆-alkyl, C₁-C₆-cycloalkyl, alkenyl;
R⁴ is halogen, hydroxy, C₁-C₆-alkyl, C₁-C₆-alkoxy, C₁-C₆-haloalkoxy, C(O)OH, C(O)OC₁-C₆-alkyl, C(O)NH₂, C(O)N(C₁-C₆-alkyl)₂ in which the two alkyl groups are independent from each other, CN;
R^{5a} is C₃-C₁₀-cycloalkyl, C₃-C₁₀-cycloalkyl-C₁-C₆-alkylen-, aryl, aryl-C₁-C₆-alkylen-, heteroaryl, heteroaryl-C₁-C₆-alkylen-, in which said cycloalkyl, aryl, heteroaryl groups are optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other
and the salts thereof.

15. A compound according to claim 14 characterised in that
x is 1
R¹ is selected from the group consisting of methyl, ethyl, cyclopropyl, ethinyl and allyl;
R⁴ is a fluorine, C₁-C₆-alkoxy, C₁-C₆-haloalkoxy, C(O)O—C₁-C₆-alkyl.

16. A compound according to claim 14 wherein
x is 2;
R¹ is selected from the group consisting of methyl, ethyl, cyclopropyl, ethinyl and allyl;
R⁴ is a fluorine, C₁-C₆-alkoxy, C₁-C₆-haloalkoxy, C(O)O—C₁-C₆-alkyl.

17. A compound according to claim 14 wherein
R⁴ is in the para or meta position on the phenyl radical of formula (Ia).

18. A compound according to claim 14 wherein
R⁴ is a fluorine or a OCF²H in the para position on the phenyl radical of formula (Ia).

19. A compound according to claim 14 wherein
R⁴ is C₁-C₆-alkoxy, C₁-C₆-haloalkoxy, C(O)O—C₁-C₆-alkyl in the meta position on the phenyl radical of formula (Ia).

20. A compound according to claim 14 wherein
R^{5a} is C₃-C₁₀-cycloalkyl, C₃-C₁₀-cycloalkyl-C₁-C₆-alkylen-, aryl or aryl-C₁-C₆-alkylen-, heteroaryl, or heteroaryl-C₁-C₆-alkylen-, in which said cycloalkyl, aryl, heteroaryl groups are optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

21. A compound according to claim 14 wherein
R^{5a} is a cyclopropyl, cyclopropyl-CH²—, cyclopentyl, cyclopentyl-CH²—, cyclohexyl, cyclohexyl-CH²—, phenyl, phenyl-CH²—, pyridyl, pyridyl-CH²—, 3,4-dihydro-2H-chromen-4-yl, optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(CH³)².

22. A compound according to Formula (Ib) 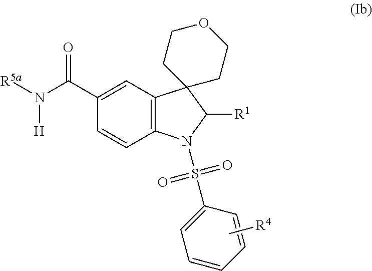
in which
R¹ is selected from the group consisting of C₁-C₆-alkyl, C₁-C₆-cycloalkyl, alkenyl;
R⁴ is halogen, hydroxy, C₁-C₆-alkyl, C₁-C₆-alkoxy, C₁-C₆-haloalkoxy, C(O)OH, C(O)O—C₁-C₆-alkyl, C(O)NH₂, C(O)N(C₁-C₆-alkyl)₂ in which the two alkyl groups are independent from each other, CN;
R^{5a} is C₃-C₁₀-cycloalkyl, C₃-C₁₀-cycloalkyl-C₁-C₆-alkylen-, aryl, aryl-C₁-C₆-alkylen-, heteroaryl, heteroaryl-C₁-C₆-alkylen-, in which said cycloalkyl, aryl, heteroaryl groups are optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other
and the salts thereof.

23. A compound according to claim 22 characterised in that
R¹ is C₁-C₆-alkyl;
R⁴ is a fluorine, C₁-C₆-alkoxy, C₁-C₆-haloalkoxy, C(O)O—C₁-C₆-alkyl.

24. A compound according to claim 22 wherein
R¹ is a methyl, ethyl, cyclopropyl, ethinyl and allyl;
R⁴ is a fluorine, C₁-C₆-alkoxy, C₁-C₆-haloalkoxy, C(O)O—C₁-C₆-alkyl.

25. A compound according to claim 22 wherein
R⁴ is in the para or meta position on the phenyl radical of formula (Ib).

26. A compound according to claim 22 wherein
R¹ is a methyl;
R⁴ is a fluorine in the para position on the phenyl radical of formula (Ib).

27. A compound according to claim 22 wherein
R^{5a} is aryl or aryl-C₁-C₆-alkylen-, heteroaryl, or heteroaryl-C₁-C₆-alkylen-, in which said aryl, heteroaryl groups are optionally substituted up to three times with a halogen, hydroxy, an C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(C₁-C₆-alkyl)₂ in which the two alkyl groups are independent from each other.

28. A compound according to claim 22 wherein
R^{5a} is a phenyl, phenyl-CH²—, pyridyl, pyridyl-CH²—, optionally substituted up to two times with a halogen, hydroxy, C₁-C₆-alkyl, C₁-C₆-haloalkyl, C₁-C₆-alkoxy, C₁-C₆-haloalkoxy, C(O)OH, C(O)O—C₁-C₆-alkyl, CN, C(O)NH₂, S(O)₂—C₁-C₆-alkyl, S(O)₂NH₂, S(O)₂N(CH³)².

29. A compound according to claim 1 that is
N-[(3-Chloropyridin-2-yl)methyl]-1-[(4-fluorophenyl)sulfonyl]-2-methyl-1,2,2′,3′,5′,6′-hexahydrospiro[indole-3,4′-pyran]-5-carboxamide,
N-(2-chlorobenzyl)-1-[(4-fluorophenyl)sulfonyl]-2-methyl-1,2,2′,3′,5′,6′-hexahydrospiro[indole-3,4′-pyran]-5-carboxamide,
1-[(4-fluorophenyl)sulfonyl]-2-methyl-N-{[3-(trifluoromethyl)pyridin-2-yl]methyl}-1,2,2′,3′,5′,6′-hexahydrospiro[indole-3,4′-pyran]-5-carboxamide,
1-[(4-fluorophenyl)sulfonyl]-2-methyl-N-[2-(trifluoromethyl)benzyl]-1,2,2′,3′,5′,6′-hexahydrospiro[indole-3,4′-pyran]-5-carboxamide,
N-[(3-chloro-5-fluoropyridin-2-yl)methyl]-1-[(4-fluorophenyl)sulfonyl]-2-methyl-1,2,2′,3′,5′,6′-hexahydrospiro[indole-3,4′-pyran]-5-carboxamide,
1-[(4-fluorophenyl)sulfonyl]-2-methyl-N-(2-pyridylmethyl)-1,2,2′,3′,5′,6′-hexahydrospiro[indole-3,4′-pyran]-5-carboxamide,
N-(4-fluorobenzyl)-1-[(4-fluorophenyl)sulfonyl]-2-methyl-1,2,2′,3′,5′,6′-hexahydrospiro[indole-3,4′-pyran]-5-carboxamide,
N-(2-cyanobenzyl)-1-[(4-fluorophenyl)sulfonyl]-2-methyl-1,2,2′,3′,5′,6′-hexahydrospiro[indole-3,4′-pyran]-5-carboxamide,
1-[(4-fluorophenyl)sulfonyl]-N-(2-mesylbenzyl)-2-methyl-1,2,2′,3′,5′,6′-hexahydrospiro[indole-3,4′-pyran]-5-carboxamide,
1-[(4-fluorophenyl)sulfonyl]-N-(3-mesylphenyl)-2-methyl-1,2,2′,3′,5′,6′-hexahydrospiro[indole-3,4′-pyran]-5-carboxamide,
N-[3-(N, N-dimethylsulfamoyl)phenyl]-1-[(4-fluorophenyl)sulfonyl]-2-methyl-1,2,2′,3′,5′,6′-hexahydrospiro[indole-3,4′-pyran]-5-carboxamide,
N-(2-chlorobenzyl)-2-cyclopropyl-1-[(4-fluorophenyl)sulfonyl]-1,2,2′,3′,5′,6′-hexahydrospiro[indole-3,4′-thiopyran]-5-carboxamide,
N-(2-chlorobenzyl)-2-cyclopropyl-1-[(4-fluorophenyl)sulfonyl]-1,2,2′,3′,5′,6′-hexahydrospiro[indole-3,4′-thiopyran]-5-carboxamide 1-oxide,
N-[(3-chloropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide,
N-(2-chloro-4-fluorobenzyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
N-[(3-chloro-5-fluoropyridin-2-yl)methyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
N-(2-chlorobenzyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
N-(2-chloro-4-fluoro-α,α-dimethylbenzyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-N-(4-fluoro-α,α-dimethylbenzyl)-1-[(4-fluorophenyl)sulfonyl]-1,2,2′,3′,5′,6′-hexahydrospiro[indole-3,4′-thiopyran]-5-carboxamide 1′,1′-dioxide,
N-[1-(2-chiorophenyl)cyclopropyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-(2-pyridylmethyl)-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-(3-mesylphenyl)-1,2,2′,3′,5′,6′-hexahydrospiro[indole-3,4′-thiopyran]-5-carboxamide 1′,1′-dioxide,
N-(3-chlorophenyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
N-[2-(2-chlorophenyl)ethyl]-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
N-[(3-chloropyridin-2-yl)methyl]-1-[(4-fluorophenyl)sulfonyl]-2-methyl-1,2,2′,3′,5′,6′-hexahydrospiro[indole-3,4′-thiopyran]-5-carboxamide 1′,1′-dioxide,
N-(2-chloro-4-fluoro-α,α-dimethylbenzyl)-1-[(4-fluorophenyl)sulfonyl]-2-methyl-1,2,2′,3′,5′,6′-hexahydrospiro[indole-3,4′-thiopyran]-5-carboxamide 1′,1′-dioxide,
N-[(3-chloro-5-fluoropyridin-2-yl)methyl]-1-[(4-fluorophenyl)sulfonyl]-2-methyl-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-(5-methylpyridin-2-yl)-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-(3-sulfamoylphenyl)-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-[(3-methylpyridin-2-yl)methyl]-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-[2-(trifluoromethyl)benzyl]-1,2,2′,3′,5′,6′-hexahydrospiro[indole-3,4′-thiopyran]-5-carboxamide 1′,1′-dioxide,
2-cyclopropyl-N-(3,4-dihydro-2H-chromen-4-yl)-1-[(4-fluorophenyl)sulfonyl]-1,2,2′,3′,5′,6′-hexahydrospiro[indole-3,4′-thiopyran]-5-carboxamide 1′,1′-dioxide,
methyl 3-[({2-cyclopropyl-1-[(4-fluorophenyl)sulfonyl]-1′,1′-dioxido-1,2,2′,3′,5′,6′-hexahydrospiro[indole-3,4′-thiopyran]-5-yl}carbonyl)amino]benzoate,
2-cyclopropyl-N-(cyclopropylmethyl)-1-[(4-fluorophenyl)sulfonyl]-1,2,2′,3′,5′,6′-hexahydrospiro[indole-3,4′-thiopyran]-5-carboxamide 1′,1′-dioxide,
N-(cyclohexylmethyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-N-[3-(dimethylsulfamoyl)phenyl]-1-[(4-fluorophenyl)sulfonyl]-1,2,2′,3′,5′,6′-hexahydrospiro[indole-3,4′-thiopyran]-5-carboxamide 1′,1′-dioxide,
N-(cyclopentylmethyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-1-[(3-methoxyphenyl)sulfonyl]-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide,
2-cyclopropyl-1-[(3-methoxyphenyl)sulfonyl]-N-[2-(trifluoromethyl)benzyl]-1,2,2′,3′,5′,6′-hexahydrospiro[indole-3,4′-thiopyran]-5-carboxamide 1′,1′-dioxide,
N-[(3-chloropyridin-2-yl)methyl]-2-cyclopropyl-1-[(3-methoxyphenyl)sulfonyl]-1,2,2′,3′,5′,6′-hexahydrospiro[indole-3,4′-thiopyran]-5-carboxamide 1′,1′-dioxide,
2-cyclopropyl-1-[(3-methoxyphenyl)sulfonyl]-N-(3-sulfamoylphenyl)-1,2,2′,3′,5′,6′-hexahydrospiro[indole-3,4′-thiopyran]-5-carboxamide 1′,1′-dioxide,
2-cyclopropyl-N-[3-(dimethylsulfamoyl)phenyl]-1-[(3-methoxyphenyl)sulfonyl]-1,2,2′,3′,5′,6′-hexahydrospiro[indole-3,4′-thiopyran]-5-carboxamide 1′,1′-dioxide,
N-(2-chlorobenzyl)-2-cyclopropyl-1-[(3-methoxyphenyl)sulfonyl]-1,2,2′,3′,5′,6′-hexahydrospiro[indole-3,4′-thiopyran]-5-carboxamide 1′,1′-dioxide,
2-cyclopropyl-1-[(3-methoxyphenyl)sulfonyl]-N-[(3-methylpyridin-2-yl)methyl]-1,2,2′,3′,5′,6′-hexahydrospiro[indole-3,4′-thiopyran]-5-carboxamide 1′,1′-dioxide,
N-(2-chloro-4-fluorobenzyl)-2-cyclopropyl-1-[(3-methoxyphenyl)sulfonyl]-1,2,2′,3′,5′,6′-hexahydrospiro[indole-3,4′-thiopyran]-5-carboxamide 1′,1′-dioxide,
2-cyclopropyl-N-(2-fluorobenzyl)-1-[(3-methoxyphenyl)sulfonyl]-1,2,2′,3′,5′,6′-hexahydrospiro[indole-3,4′-thiopyran]-5-carboxamide 1′,1′-dioxide,
methyl 3-[({2-cyclopropyl-1-[(3-methoxyphenyl)sulfonyl]-1′,1′-dioxido-1,2,2′,3′,5′,6′-hexahydrospiro[indole-3,4′-thiopyran]-5-yl}carbonyl)amino]benzoate,
3-[({2-cyclopropyl-1-[(4-fluorophenyl)sulfonyl]-1′,1′-dioxido-1,2,2′,3′,5′,6′-hexahydrospiro[indole-3,4′-thiopyran]-5-yl}carbonyl)amino]benzoic acid,
3-[({2-cyclopropyl-1-[(3-methoxyphenyl)sulfonyl]-1′,1′-dioxido-1,2,2′,3′,5′,6′-hexahydrospiro[indole-3,4′-thiopyran]-5-yl}carbonyl)amino]benzoic acid,
N-(3-carbamoylphenyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-[(3-fluoropyridin-2-yl)methyl]-1,2,2′,3′,5′,6′-hexahydrospiro[indole-3,4′-thiopyran]-5-carboxamide 1′,1′-dioxide,
2-cyclopropyl-N-[(3-fluoropyridin-2-yl)methyl]-1-[(3-methoxyphenyl)sulfonyl]-1,2,2′,3′,5′,6′-hexahydrospiro[indole-3,4′-thiopyran]-5-carboxamide 1′,1′-dioxide,
methyl 3-({5-[N-(2-chlorobenzyl)carbamoyl]-1′,1′-dioxido-2-(prop-2-en-1-yl)-1,2,2′,3′,5′,6′-hexahydrospiro[indole-3,4′-thiopyran]-1-yl}sulfonyl)benzoate,
methyl 3-({5-[N-(2-chlorobenzyl)carbamoyl]-1′,1′-dioxido-2-vinyl-1,2,2′,3′,5′,6′-hexahydrospiro[indole-3,4′-thiopyran]-1-yl}sulfonyl)benzoate,
3-({5-[(2-chlorobenzyl)carbamoyl]-1′,1′-dioxido-2-(prop-2-en-1-yl)-2′,3′,5′,6′-tetrahydrospiro[indole-3,4′-thiopyran]-1(2H)-yl}sulfonyl)benzoic acid,
N-[(3-chloropyridin-2-yl)methyl]-1-[(4-fluorophenyl)sulfonyl]-2-(prop-2-en-1-yl)-1,2,2′,3′,5′,6′-hexahydrospiro[indole-3,4′-thiopyran]-5-carboxamide 1′,1′-dioxide,
methyl 3-[({1-[(4-fluorophenyl)sulfonyl]-1′,1′-dioxido-2-(prop-2-en-1-yl)-1,2,2′,3′,5′,6′-hexahydrospiro[indole-3,4′-thiopyran]-5-yl}carbonyl)amino]benzoate,
3-[({1-[(4-fluorophenyl)sulfonyl]-1′,1′-dioxido-2-(prop-2-en-1-yl)-1,2,2′,3′,5′,6′-hexahydrospiro[indole-3,4′-thiopyran]-5-yl}carbonyl)amino]benzoic acid,
methyl 3-({5-[(2-chlorobenzyl)carbamoyl]-2-cyclopropyl-1′,1′-dioxido-2′,3′,5′,6′-tetrahydrospiro[indole-3,4′-thiopyran]-1 (2H)-yl}sulfonyl)benzoate,
3-({5-[(2-chlorobenzyl)carbamoyl]-2-cyclopropyl-1′,1′-dioxido-2′,3′,5′,6′-tetrahydrospiro[indole-3,4′-thiopyran]-1 (2H)-yl}sulfonyl)benzoic acid,
N-(3-{[bis(dimethylamino)methylidene]sulfamoyl}phenyl)-2-cyclopropyl-1-[(3-methoxyphenyl)sulfonyl]-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-(1,2-oxazol-3-yl)-1,2,2′,3′,5′,6′-hexahydrospiro[indole-3,4′-thiopyran]-5-carboxamide 1′,1′-dioxide,
N-(3-{[bis(dimethylamino)methylidene]sulfamoyl}phenyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-{[5-(trifluoromethyl)pyridin-2-yl]methyl}-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-{3-[(5-methyl-1,2-oxazol-3-yl)sulfamoyl]phenyl}-1,2,2′,3′,5′,6′-hexahydrospiro[indole-3,4′-thiopyran]-5-carboxamide 1′,1′-dioxide,
N-(2-chlorophenyl)-2-cyclopropyl-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-N-[2-(difluoromethyl)benzyl]-1-[(4-fluorophenyl)sulfonyl]-1,2,2′,3′,5′,6′-hexahydrospiro[indole-3,4′-thiopyran]-5-carboxamide 1′,1′-dioxide,
2-cyclopropyl-1-[(4-fluorophenyl)sulfonyl]-N-(2-hydroxybenzyl)-1,2,2′,3′,5′,6′-hexahydrospiro[indole-3,4′-thiopyran]-5-carboxamide 1′,1′-dioxide,
N-[(3-chloropyridin-2-yl)methyl]-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide,
N-(5-chloropyridin-3-yl)-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide,
1-[(4-cyanophenyl)sulfonyl]-2-cyclopropyl-N-[2-(trifluoromethyl)benzyl]-1,2,2′,3′,5′,6′-hexahydrospiro[indole-3,4′-thiopyran]-5-carboxamide 1′,1′-dioxide,
1-[(4-cyanophenyl)sulfonyl]-2-cyclopropyl-N-(1,3-oxazol-2-yl)-1,2,2′,3′,5′,6′-hexahydrospiro[indole-3,4′-thiopyran]-5-carboxamide 1′,1′-dioxide,
N-(2-chlorophenyl)-1-[(4-cyanophenyl)sulfonyl]-2-cyclopropyl-1,2,2′,3′,5′,6′-hexahydrospiro[indole-3,4′-thiopyran]-5-carboxamide 1′,1′-dioxide,
1-[(4-cyanophenyl)sulfonyl]-2-cyclopropyl-N-(2-fluorophenyl)-1,2,2′,3′,5′,6′-hexahydrospiro[indole-3,4′-thiopyran]-5-carboxamide 1′,1′-dioxide,
N-(2-chlorobenzyl)-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide,
N-(5-chloropyridin-3-yl)-1-[(3-cyanophenyl)sulfonyl]-2-cyclopropyl-1,2,2′,3′,5′,6′-hexahydrospiro[indole-3,4′-thiopyran]-5-carboxamide 1′,1′-dioxide,
1-[(3-cyanophenyl)sulfonyl]-2-cyclopropyl-N-(1,3-oxazol-2-yl)-1,2,2′,3′,5′,6′-hexahydrospiro[indole-3,4′-thiopyran]-5-carboxamide 1′,1′-dioxide,
1-[(3-cyanophenyl)sulfonyl]-2-cyclopropyl-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide,
1-[(3-cyanophenyl)sulfonyl]-2-cyclopropyl-N-(1,2-oxazol-3-yl)-1,2,2′,3′,5′,6′-hexahydrospiro[indole-3,4′-thiopyran]-5-carboxamide 1′,1′-dioxide,
N-(2-chlorobenzyl)-2-cyclopropyl-1-{[3-(trifluoromethoxy)phenyl]sulfonyl}-1,2,2′,3′,5′,6′-hexahydrospiro[indole-3,4′-thiopyran]-5-carboxamide 1′,1′-dioxide,
methyl 3-{[(2-cyclopropyl-1′,1′-dioxido-1-{[3-(trifluoromethoxy)phenyl]sulfonyl}-1,2,2′,3′,5′,6′-hexahydrospiro[indole-3,4′-thiopyran]-5-yl)carbonyl]amino}benzoate,
3-{[(2-cyclopropyl-1′,1′-dioxido-1-{[3-(trifluoromethoxy)phenyl]sulfonyl}-1,2,2′,3′,5′,6′-hexahydrospiro[indole-3,4′-thiopyran]-5-yl)carbonyl]amino}benzoic acid,
2-cyclopropyl-1-{[3-(trifluoromethoxy)phenyl]sulfonyl}-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide,
2-cyclopropyl-N-(5-methylpyridin-3-yl)-1-{[3-(trifluoromethoxy)phenyl]sulfonyl}-1,2,2′,3′,5′,6′-hexahydrospiro[indole-3,4′-thiopyran]-5-carboxamide 1′,1′-dioxide,
N-(2-chlorobenzyl)-2-cyclopropyl-1-{[3-(difluoromethoxy)phenyl]sulfonyl}-1,2,2′,3′,5′,6′-hexahydrospiro[indole-3,4′-thiopyran]-5-carboxamide 1′,1′-dioxide,
2-cyclopropyl-1-{[3-(difluoromethoxy)phenyl]sulfonyl}-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide,
N-(5-chloropyridin-3-yl)-2-cyclopropyl-1-{[3-(difluoromethoxy)phenyl]sulfonyl}-1,2,2′,3′,5′,6′-hexahydrospiro[indole-3,4′-thiopyran]-5-carboxamide 1′,1′-dioxide,
methyl 3-{[(2-cyclopropyl-1-{[3-(difluoromethoxy)phenyl]sulfonyl}-1′,1′-dioxido-1,2,2′,3′,5′,6′-hexahydrospiro[indole-3,4′-thiopyran]-5-yl)carbonyl]amino}benzoate,
3-{[(2-cyclopropyl-1-{[3-(difluoromethoxy)phenyl]sulfonyl}-1′,1′-dioxido-1,2,2′,3′,5′,6′-hexahydrospiro[indole-3,4′-thiopyran]-5-yl)carbonyl]amino}benzoic acid,
2-cyclopropyl-1-{[4-(difluoromethoxy)phenyl]sulfonyl}-N-[2-(difluoromethyl)benzyl]-1,2,2′,3′,5′,6′-hexahydrospiro[indole-3,4′-thiopyran]-5-carboxamide 1′,1′-dioxide,
2-cyclopropyl-1-{[4-(difluoromethoxy)phenyl]sulfonyl}-N-[2-(trifluoromethyl)benzyl]-1,2,2′,3′,5′,6′-hexahydrospiro[indole-3,4′-thiopyran]-5-carboxamide 1′,1′-dioxide,
2-cyclopropyl-1-{[4-(difluoromethoxy)phenyl]sulfonyl}-N-{[3-(trifluoromethyl)pyridin-2-yl]methyl}-1,2,2′,3′,5′,6′-hexahydrospiro[indole-3,4′-thiopyran]-5-carboxamide 1′,1′-dioxide,
1-[(4-carbamoylphenyl)sulfonyl]-N-(2-chlorobenzyl)-2-cyclopropyl-1,2,2′,3′,5′,6′-hexahydrospiro[indole-3,4′-thiopyran]-5-carboxamide 1′,1′-dioxide,
1-[(4-carbamoylphenyl)sulfonyl]-2-cyclopropyl-N-{3-[(I-methylpyrrolidin-2-ylidene)sulfamoyl]phenyl}-1,2,2′,3′,5′,6′-hexahydrospiro[indole-3,4′-thiopyran]-5-carboxamide 1′,1′-dioxide,
1-[(4-carbamoylphenyl)sulfonyl]-2-cyclopropyl-N-[3-(1,3-thiazol-2-ylsulfamoyl)phenyl]-1,2,2′,3′,5′,6′-hexahydrospiro[indole-3,4′-thiopyran]-5-carboxamide 1′,1′-dioxide,
N-{2-cyclopropyl-1-[(4-fluorophenyl)sulfonyl]-1′,1′-dioxido-1,2,2′,3′,5′,6′-hexahydrospiro[indole-3,4′-thiopyran]-5-yl}cyclopropanecarboxamide,
N-{2-cyclopropyl-1-[(4-fluorophenyl)sulfonyl]-1′,1′-dioxido-1,2,2′,3′,5′,6′-hexahydrospiro[indole-3,4′-thiopyran]-5-yl}cyclohexanecarboxamide, or
N-{2-cyclopropyl-1-[(4-fluorophenyl)sulfonyl]-1′,1′-dioxido-1,2,2′,3′,5′,6′-hexahydrospiro[indole-3,4′-thiopyran]-5-yl}cyclopentanecarboxamide,
and salts thereof.

30-32. (canceled)

33. A method for contraception or for treatment of endometriosis, uterine fibroids, polycystic ovarian disease, hirsutism, precocious puberty, gonadal steroid-dependent neoplasia such as cancers of the prostate, breast and ovary, gonadotrope pituitary adenomas, sleep apnea, irritable bowel syndrome, premenstrual syndrome, benign prostatic hypertrophy, contraception, infertility, assisted reproductive therapy such as in vitro fertilization, in the treatment of growth hormone deficiency and short stature, and in the treatment of systemic lupus erythematosus, the method comprising the step of administering an effective amount of a compound according to claim 1 to a patient in need thereof.
